(19) United States
(12) Patent Application Publication
(10) Pub. No.: US 2021/0115211 A1
(43) Pub. Date: Apr. 22, 2021

(54) NANOSTRUCTURED POLYMER-BASED COMPOSITIONS AND METHODS TO FABRICATE THE SAME

(71) Applicant: The Board of Trustees of the University of Illinois, Urbana, IL (US)

(72) Inventors: Jean Paul ALLAIN, Champaign, IL (US); Shuquan CHANG, Nanjing, Jiangsu (CN); Zachariah KOYN, Champaign, IL (US); Ana Fatima CIVANTOS FERNANDEZ, Urbana, IL (US); Sandra Liliana ARIAS SUAREZ, Urbana (IL)

(73) Assignee: The Board of Trustees of the University of Illinois, Urbana, IL (US)

(21) Appl. No.: 16/500,662

(22) PCT Filed: Apr. 6, 2018
(86) PCT No.: PCT/US18/26606

§ 371 Date: Oct. 3, 2019

Related U.S. Application Data
(60) Provisional application No. 62/483,105, filed on Apr. 7, 2017.
(60) Provisional application No. 62/483,074, filed on Apr. 7, 2017.
(60) Provisional application No. 62/556,048, filed on Sep. 8, 2017.
(60) Provisional application No. 62/556,120, filed on Sep. 8, 2017.

(51) Int. Cl. C08J 7/06; C08K 3/04; C08K 3/08; C08K 3/22; C08K 3/30; C08K 3/015; C08J 3/07; C08J 3/28; C08J 7/056; C08J 5/18; A61L 31/02; A61L 31/14

(52) U.S. Cl. CPC C08J 7/06; C08K 3/04; C08K 3/08; C08K 3/22; C08K 3/30; C08K 3/015; B82Y 30/00; C08J 3/28; C08J 7/056; C08J 5/18; A61L 31/022; A61L 31/146; C08J 3/07

(57) ABSTRACT

Provided herein are methods for the controlled, independent modification of the surface of polymer-based materials and compositions generated thereby. The methods include use of low temperature plasma for surface modification. The methods allow for the alteration of multiple surface characteristics including generation of precise nanostructures, morphology, crystallography and chemical composition for increased biocompatibility, for example, hydrophilicity, steric hindrance, anti-inflammatory properties and/or anti-bacterial properties.

### BACKGROUND OF INVENTION

The merging of metal oxides and polymers has a number of interesting potential applications that rely on the wettability, optical, and electronic properties of the surface. One challenge in the fabrication of these dissimilar materials is that the heat often used to create oxide nanostructures results in the thermal decomposition of the polymer. This requires creative approaches to successfully merge these materials. Many current approaches involve the separate creation of metal oxide nanostructures, followed by some process of embedding them in an uncured polymer. Previous work has shown that ion beams have been used to sputter deposit metals, pattern polycrystalline metals, controllably oxidize metal surfaces, and induce chemical changes in the surfaces of polymers.

Metal nanoparticles have attracted much attention for their unusual chemical and physical properties. Gold nanoparticles have been used in many fields such as biotechnology, optics, electronics, catalysis, and sensors. Silver nanoparticles have also been widely used in sensors, antibacterial and photocatalytic areas. The synthesis of nanoparticles with different chemical composition, size distribution, and controlled mono dispersion is an important area of research in nanotechnology. Many methods such as vapor deposition, solvent-thermal, sol-gel, electrochemistry and microwave have been developed to fabricate nanoparticles. The stability and functional properties of nanoparticles are critical to their application, which are traditionally determined by the coatings. Bacterial nanocellulose (BNC) and chitosan (CS) are fascinating and renewable natural nanomaterial characterized by favorable properties such as remarkable mechanical properties, porosity, water absorbency, moldability, biodegradability and excellent biological affinity. Intensive research and exploration in the past few decades on BNC/CS nanomaterials mainly focused on their biosynthetic process to achieve the low cost preparation and application in medical, food, advanced acoustic diaphragms, and other fields. These investigations have led to the emergence of more diverse potential applications expiating the functionality of BNC/CS nanomaterials. There is a great demand for multifunctional nanomaterials in the biomedical, new energy and other areas. Recently, Ag or Au nanoparticle-modified BNC have been fabricated using traditional chemical methods and exploited their application in antibacterial, detector, sensor, catalysis, and imaging.

Plasma has also been used to alter chemical and mechanical properties of substances. However, known methods of plasma treatment (e.g. kinetic roughening) are imprecise and provide little control over the plasma surface interaction. By providing precise porous and/or nanopatterned regions to the materials. Nanopatterned surfaces have been obtained mostly by bottom-up and top-down techniques on model materials given the difficulty in high-fidelity control of clinically-relevant surfaces and of complex 3D systems. Furthermore, no current nanoscale modification method exists that can control both surface chemistry and topography independently.

Biofilm formation has important public health implications. Drinking water systems are known to harbor biofilms, even though these environments often contain disinfectants. Any system providing an interface between a surface and a fluid has the potential for biofilm development. Biofilms are a constant problem in food processing environments. Food processing involves fluids, solid material and their combination. As an example, milk processing facilities provide fluid conduits and areas of fluid residence on surfaces. Meat processing and packing facilities are in like manner susceptible to biofilm formation. Nonmetallic and metallic surfaces can be affected. Biofilms in meat processing facilities have been detected on rubber “fingers,” plastic curtains, conveyor belt material, evisceration equipment and stainless steel surfaces. Controlling biofilms and microorganism contamination in food processing is hampered by the additional need that the agent used not affect the taste, texture or aesthetics of the product.

There exists, therefore, a need to be able to render general surfaces, as well as a need in the art for improved methods for surface modification of substrates including synthetic and natural polymers at the nanometer level.

### SUMMARY OF THE INVENTION

In one embodiment, the instant invention includes a polymer composition which includes a polymer substrate having a surface; a plurality of metal, metal oxide, or carbon allotrope nanoparticles disposed on said surface. The surface of the polymer composition, in some embodiments, has a plurality of nanoscale domains characterized by a surface geometry providing a selected function. Each of the nanoscale domains has at least one lateral spatial dimension selected over the range of 10 nm to 1 μm and a vertical spatial dimension less than 200 nm.

In one embodiment the instant invention includes a polymer composition which includes a polymer substrate having a surface; a plurality of metal, metal oxide nanoparticles, or carbon allotrope nanoparticles disposed on said surface. The surface has a plurality of nanoscale domains characterized by a surface geometry providing a selected function; and nanoscale domains are generated by exposing said surface to one or more directed energetic particle beam characterized by one or more beam properties.

In embodiments, the polymer may be a polysaccharide biopolymer or a synthetic polymer. The polysaccharide biopolymer includes cellulose, such as bacterial nanocellulose, nanocellulose, and a cellulose derivative. The polysaccharide biopolymer also includes chitin; a dextran; chitosan; and combinations thereof.

In embodiments, the synthetic polymer can include a polyolefin; a silicone; a polyacrylate or polymethacrylate; a polyester; a polyether; a polyamide, and a polyurethane. The polyolefin may be selected from polypropylene, polyethylene, poly(tetrafluoroethylene) and poly(vinyl chloride. The silicone may include poly(dimethyl siloxane). The polyacrylate or polymethacrylate may include poly(methyl methacrylate), poly(hydroxyethyl methacrylate). The polyester may include poly(ethylene terephthalate), poly(glycolic acid), poly-lactic acid, polydioxanone; or wherein the synthetic polymer is a polyether selected from the group consisting of polyether ether ketone and polyether sulfone.

In embodiments, the selected function may be an activity related to at least one biological or physical property, relative to a polymer composition not having said plurality of nanoscale domains characterized by said nanofeatured surface geometry. In embodiments, the activity can be an enhancement of a biological property selected from the group consisting of cell adhesion activity, cell proliferation activity, cell in-migration activity, cell differentiation activity, anti-bacterial activity, bactericidal activity, anti-inflammatory activity, osseointegration activity, hemocompatibility activity, osseoconduction activity, osseoinduction activity, reduction of immunoresponse, and combinations thereof.

The enhancement of the biological property is equal to or greater than about 100% to about greater than or equal to 500%. In embodiments, the enhancement is equal to or greater than 10%, 20%, 50%, 70%, 100%, 150%, 200%, 300%, 400%, 500%, 1000% (i.e., 10×), 20×, or 50×. Such enhancement may be measured by methods known in the art for testing for the relevant biological properties.

In another embodiment, the selected function may be an activity. This activity can include an enhancement of a physical property selected from the group consisting of surface hydrophilicity, surface free energy, surface hydrophobicity, sensing, drug transport, surface acidity, surface basicity, and combinations thereof. In embodiments, the enhancement is equal to or greater than 10%, 20%, 50%, 70%, 100%, 150%, 200%, 300%, 400%, 500%, 1000% (i.e., 10×), 20×, or 50×. Such enhancement may be measured by methods known in the art for testing for the relevant activities.

In some embodiments, the surface geometry is spatial distribution of relief features, recessed features, localized regions characterized by a selected composition, phase, crystallographic texture, or any combination of these. In one embodiment, the surface geometry is a periodic or semi-periodic spatial distribution of said nanoscale domains; or the surface geometry is a selected topology, topography, morphology, texture or any combination of these.

In embodiments, the nanoscale domains can include nanopillars, nanowalls, nanorods, nanoplates, nanoripples, surface porous structure, or any combination thereof having lateral spatial dimensions selected over the range of 10 nm to 1 μm and vertical spatial dimensions of less than or equal to 200 nm and wherein said nanoscale domains are separated from one another by a distance of 50-500 nm. In some embodiments, the nanopillars, nanowalls, nanorods, nanoplates, nanoripples, surface porous structure, or combination thereof, are inclined towards a direction oriented along a selected axis relative to said surface.

In some embodiments, nanoscale domains include nanopillars, nanowalls, nanorods, nanoplates, nanoripples, surface porous structure having lateral spatial dimensions selected over the range of 10 nm to 1 μm. Nanoscale domains may include nanopillars. The lateral spatial dimensions can be between 10 nm-50 nm, between 10 nm-100 nm, between 10 nm-500 nm; or between 20 nm and 50 nm, between 20 nm and 100 nm, between 20 nm and 200 nm, between 20 nm and 500 nm, or between 20 nm and 1000 nm; or between 50 nm and 100 nm, between 50 nm and 200 nm, between 50 nm and 500 nm, or between 50 nm and 1000 nm; or between 100 nm and 200 nm, between 100 nm and 500 nm, or between 100 nm and 1000 nm; or between 200 nm and 500 nm, or between 200 nm and 1000 nm; or between 500 nm and 1000 nm.

In some embodiments, nanoscale domains include nanopillars, nanowalls, nanorods, nanoplates, nanoripples, surface porous structure having vertical spatial dimensions selected over the range of 1 nm to 200 μm. Vertical spatial dimensions can include between 1 nm and 10 nm, between 1 nm and 20 nm, between 1 nm and 50 nm, between 1 nm and 100 nm, between 1 nm and 200 nm; or between 5 nm and 10 nm, between 5 nm and 20 nm, between 5 nm and 50 nm, between 5 nm and 100 nm, between 5 nm and 200 nm; or between 10 nm and 20 nm, between 10 nm and 50 nm, between 10 nm and 100 nm, between 10 nm and 200 nm; or between 20 nm and 50 nm, between 20 nm and 100 nm, between 20 nm and 200 nm; or between 50 nm and 100 nm, between 50 nm and 200 nm; or between 100 nm and 200 nm.

In some embodiments, nanoscale domains include nanopillars, nanowalls, nanorods, nanoplates, nanoripples, surface porous structure, where an individual nanoscale domain is separated from another individual nanoscale domain selected over the range of 50 nm to 500 μm. Separation spatial dimensions can include between 50 nm and 100 nm, between 50 nm and 200 nm, between 50 nm and 500 nm; between 100 nm and 200 nm, between 100 nm and 500 nm, between 200 nm and 500 nm.

In some embodiments, nanoscale domains include nanoripples. Nanoripples may also be described as having a nanostructure that extends in length, i.e., has a lengthwise dimension (along the length of an individual ripple) as well as a lateral dimension (perpendicularly across the length of an individual ripple). The lengthwise dimension of a nanoripple may be between about 0.1 μm to about 10 μm. Lengthwise spatial dimensions can include between 0.1 μm and 0.5 μm, between 0.1 μm and 1 μm, between 0.1 μm and 2 μm; between 0.1 μm and 5 μm; between 0.5 μm and 1 μm, between 0.5 μm and 2 μm; between 0.5 μm and 5 μm, between 0.5 μm and 10 μm; between 1 μm and 2 μm; between 1 μm and 5 μm, between 1 μm and 10 μm; or between 2 μm and 5 μm, between 5 μm and 10 μm. The peak to peak dimension of the plurality of nanoripples can be between 100 nm and 300 nm.

The dimensional numbers given above may be averages, geometric mean, or a confidence interval of 90% or 95%.

In embodiments, the metal or metal oxide nanoparticles comprise gold nanoparticles, silver nanoparticles, zinc sulfide nanoparticles, zinc oxide nanoparticles, copper nanoparticles, platinum nanoparticles, cobalt nanoparticles, cobalt ferrite nanoparticles, ferric oxide nanoparticles, yttrium nanoparticles, zirconium nanoparticles, ruthenium nanoparticles, palladium nanoparticles, or any combinations or oxides thereof.

In embodiments, the nanoparticles have a diameter of between 10 nm and about 500 nm. The nanoparticles may have a diameter of between 10 nm and 20 nm, between 10 nm and 50 nm, between 10 nm and 100 nm, between 10 nm and 200 nm; or between 10 nm and about 500 nm. The nanoparticles may have a diameter of between 20 nm and 50 nm, between 20 nm and 100 nm, between 100 nm and 200 nm; 10 nm and about 500 nm. The nanoparticles may have a diameter of between 50 nm and 100 nm, between 50 nm and 200 nm, or between 100 nm and 200 nm. Alternatively, or in addition to, the nanoparticles may have dimensions between 200 nm and 500 nm, between 250 nm and 500 nm, between 300 nm and 500 nm, between 400 nm and 500 nm; between 200 nm and 400 nm; between 250 nm and 400 nm; between 300 nm and 400 nm; or between 400 nm and 500 nm.

In one embodiment, the present invention includes a polysaccharide biopolymer with anti-bacterial and super-hydrophilic properties. In this embodiment, the nanoscale domains include nanopillars and the polysaccharide biopolymer comprises chitosan or bacterial nanocellulose. In this embodiment the metal, metal oxide, or carbon allotrope nanoparticles include one or more of zinc sulfide nanoparticles, gold nanoparticles or silver nanoparticles, and the selected function includes enhanced antibacterial properties or enhanced hydrophilicity. In embodiments, the nanopillars have lateral spatial dimensions selected over the range of 10 nm to 1 μm and vertical spatial dimensions of less than or equal to 200 nm and wherein said nanoscale domains are separated from one another by a distance of 50-500 nm, and wherein the nanoparticles have a diameter of between about 10-50 nm.

In one embodiment, the present invention relates to a polymer composition which includes a polysaccharide biopolymer with anti-bacterial and super-hydrophilic properties that may be created, optionally, by liquid plasma synthesis. In this embodiment, the nanoscale domains may comprise surface porous structure. The polysaccharide biopolymer may include chitosan or bacterial nanocellulose, and said metal, metal oxide, or carbon allotrope nanoparticles may include zinc sulfide nanoparticles, gold nanoparticles or silver nanoparticles. The selected function is enhanced antibacterial properties or enhanced hydrophilicity. In embodiments, the surface porous structure can have lateral spatial dimensions selected over the range of 50 nm to 500 μm and vertical spatial dimensions of between 10 and 50 nm and wherein the nanoparticles have a diameter of between about 10-50 nm.

In another embodiment, the present invention relates to a polymer composition which includes a polymer composition comprising metal oxide nanoparticles on nanoscale patterned flexible synthetic polymer substrates. In embodiments, the nanoscale domains comprise nanoripples having lengthwise spatial dimensions selected over the range of 0.5 microns to 10 microns, vertical dimension of between 50 nm to about 200 nm, peak to peak spatial dimensions of between about 100 nm to 300 nm, and wherein the nanoparticles have a diameter of between about 10 and 50 nm and/or between 250-500 nm. In embodiments, the metal or metal oxide nanoparticles comprise gold nanoparticles, silver nanoparticles, zinc sulfide nanoparticles, zinc oxide nanoparticles, copper nanoparticles, platinum nanoparticles, cobalt nanoparticles, cobalt ferrite nanoparticles, ferric oxide nanoparticles, yttrium nanoparticles, zirconium nanoparticles, ruthenium nanoparticles, palladium nanoparticles, or any combinations thereof. In some embodiments, the metal or metal oxide nanoparticles comprise zinc oxide nanoparticles.

In one embodiment, the polymer composition comprising metal oxide nanoparticles on nanoscale patterned flexible synthetic polymer substrates have nanoscale domains which may include nanoripples; wherein said polymer comprises poly(dimethyl siloxane); wherein said metal, metal oxide, or carbon allotrope nanoparticles comprise zinc oxide nanoparticles; and wherein said selection function is enhanced hydrophilicity. The nanoripples may have a lengthwise spatial dimension selected over the range of 0.5 microns to 10 microns, vertical dimension of between 50 nm to about 200 nm, peak to peak spatial dimensions of between about 100 nm to 300 nm, and wherein the nanoparticles have a diameter of between about 10 and 50 nm and/or between 250-500 nm.

In some embodiments, the polymer composition may be included as a component of a medical device, a sensor, a catalyst, or an imaging system. Exemplary medical device include a surgical material, an implant, a catheter, a wound suture, an artificial tendon, a pacemaker, a cochlear implant, a neural implant, intravenous tubing, a surgical sponge, gauze, a needle, a syringe, a cosmetic silicone implant or a cosmetic silicone prosthetic. In some embodiments, the component of a medical device is a coating, a connection, or a wire.

As noted above, the polymer composition may be rendered anti-bacterial by the treatments described herein. On many surfaces exposed to the environment, there is the risk that a microbial biofilm may form on a surface. The compositions of the invention may be used together with any surface. The surface is not limited and includes any surface on which a microorganism may occur, particularly a surface exposed to water or moisture. Treating surfaces to avoid films of antimicrobial compounds or manufacturing with them the working surfaces of laboratories (clinical, microbiological, water analysis, food), of businesses handling fresh food (butchers, fishmongers, etc.), of hospital buildings and health centers, to mention just a few examples, guarantees the suitable hygienic conditions for development of the work and eliminates the risk of contamination and infections.

Such inanimate surfaces exposed to microbial contact or contamination include in particular any part of: food or drink processing, preparation, storage or dispensing machinery or equipment, air conditioning apparatus, industrial machinery, e.g. in chemical or biotechnological processing plants, storage tanks and medical or surgical equipment. Any apparatus or equipment for carrying or transporting or delivering materials, which may be exposed to water or moisture is susceptible to biofilm formation. Such surfaces will include particularly pipes (which term is used broadly herein to include any conduit or line). Representative inanimate or abiotic surfaces include, but are not limited to food processing, storage, dispensing or preparation equipment or surfaces, tanks, conveyors, floors, drains, coolers, freezers, equipment surfaces, walls, valves, belts, pipes, air conditioning conduits, cooling apparatus, food or drink dispensing lines, heat exchangers, boat hulls or any part of a boat's structure that is exposed to water, dental waterlines, oil drilling pipe, hose, pump, blower, contact lenses and storage cases. As noted above, medical or surgical equipment or devices represent a particular class of surface on which a biofilm may form. This may include any kind of line, including catheters (e.g. central venous and urinary catheters), prosthetic devices e.g., heart valves, artificial joints, false teeth, dental crowns, dental caps and soft tissue implants (e.g. breast, buttock and lip implants). Any kind of implantable (or “in-dwelling”) medical device is included (e.g. stents, intrauterine devices, pacemakers, intubation tubes, prostheses or prosthetic devices, lines or catheters). An “in-dwelling” medical device may include a device in which any part of it is contained within the body, i.e. the device may be wholly or partly in-dwelling. Plastic materials with antimicrobial properties can also be used in manufacturing handles, handlebars, handgrips and armrests of public transport elements, in rails and support points in places widely used, in the manufacturing of sanitary ware for public and mass use, as well as in headphones and microphones of telephones and audio systems in public places; kitchen utensils and food transport, all with the purpose of reducing the risk of propagation of infections and diseases.

In some embodiments, the directed energetic particle beam includes a broad beam, focused beam, asymmetric beam, thermalized plasma in liquid or any combination of these. The beam properties of the directed energetic particle beam includes intensity, fluence, energy, flux, incident angle, ion composition, neutral composition or any combinations thereof. The directed energetic particle beam may comprise one or more ions, neutrals or combinations thereof.

Methods for fabricating the compositions of the invention, in one embodiment, includes the following steps. A polysaccharide biopolymer is provided together with a metal salt. The polysaccharide polymer may be provided in a solution or in the form of a dried film, whereas the metal salt is optionally in the form of a dissolved solution. After an incubation period, the polysaccharide polymer which has been incubated with metal salt may be optionally dried into a film. The film comprises a substrate having a surface. A directed energetic particle beam may be beamed onto said dried substrate surface, thereby generating a plurality of nanoscale domains and metal nanoparticles on said surface. The directed energetic particle beam has one or more beam properties selected to generate said plurality of nanoscale domains characterized by a surface geometry providing a selected function as described herein.

The method may include immersing the dried substrate in a liquid, wherein the directed energetic particle beam is directed onto said substrate surface through the liquid.

The directed energetic particle beam can include a broad beam, focused beam asymmetric beam or any combination of these. The step of directing said directed energetic particle beam onto said substrate surface can include directed irradiation synthesis (DIS), directed plasma nanosynthesis (DPNS), Direct Seeded Directed Plasma Nanosynthesis (DSDPNS), DSPNS (directed soft plasma nanosynthesis) or any combination of these. The one or more beam properties can include intensity, fluence, energy, flux, incident angle, ion composition, neutral composition or any combinations thereof.

In an embodiment for certain applications, the step of directing the directed energetic particle beam onto the substrate surface is achieved using a method other than directed irradiation synthesis (DIS). For example, the invention, includes methods of fabricating a bioactive polymer substrate wherein directed plasma nanosynthesis (DPNS), direct seeded plasma nanosynthesis (DSPNS), DSPNS (directed soft plasma nanosynthesis) or any combination of these techniques is used to carry out the step of directing the directed energetic particle beam onto the substrate surface to generate a plurality of nanoscale domains characterized by a surface geometry providing a selected multifunctional bioactivity. Accordingly, one of skill in the art will readily understand that certain applications and materials of the invention are achieved using methods that do not include processing via directed irradiation synthesis (DIS).

The directed energetic particle beam can include one or more ions, neutrals or combinations thereof. The ions, for example, may include krypton (Kr) ions, argon (Ar) ions, oxygen (O) ions, or a combination thereof. The beam properties may include incident angle and the incident angle may be selected from the range of 0° to 90°.

The one or more beam properties can comprise fluence which can be selected from the range of 1×10¹⁶ ions/cm² to 1×10¹⁹ ions/cm². The one or more beam properties can comprise energy which can be selected from the range of 0.01 eV to 10 keV. For DPNS, the neutral and reactive beams may be combined at energies between 50-1000 eV with multiplexing at the surface. When the reactive beam is in the hyperthermal regime or energies of 0.1 to 10 eV, it is then defined as DSPNS (directed soft plasma nanosynthesis). The metal salt may include HAuCl₄, AgNO₃.

In one embodiment, the method includes a method of fabricating a polymer substrate composition as follows. The method includes providing a synthetic polymer substrate, providing a solid metal or metal oxide source target material, and directing at least one directed energetic particle beam onto said target material surface, thereby generating a sputtered beam of target material directed onto the surface of the synthetic polymer substrate; and directing a second directed energetic particle beam onto said substrate surface. This method generates a plurality of nanoscale domains and metal or metal oxide nanoparticles on said substrate surface. The directed energetic particle beam(s) may have one or more beam selected to generate said plurality of nanoscale domains characterized by a surface geometry providing a selected function. In embodiments, the first or second directed energetic particle beam is independently a broad beam, focused beam asymmetric beam or any combination of these.

In embodiments, the target may be a metal target selected from the group consisting of zinc, gold, silver, copper, platinum, cobalt, cobalt, yttrium, zirconium, ruthenium, palladium, or any combinations thereof.

In one embodiment, the present invention includes a polymer composition which may include a polymer substrate having a surface; wherein said surface has a plurality of nanoscale domains characterized by a surface geometry providing a selected multifunctional bioactivity comprising enhancement in cell adhesion activity or cell proliferation. In this embodiment, each of said nanoscale domains has at least one lateral spatial dimension selected over the range of 3 nm to 1 μm and a vertical spatial dimension 50 nm to 1000 nm.

In another embodiment, the present invention includes a polymer composition which may include a polymer substrate having a surface; wherein said surface has a plurality of nanoscale domains characterized by a surface geometry providing a selected multifunctional bioactivity comprising enhancement in cell adhesion activity or cell proliferation. In this embodiment, the nanoscale domains are generated by exposing said surface to one or more directed energetic particle beam characterized by one or more beam properties.

In an embodiment, the function is an activity related to at least one biological or physical property, relative to a polymer composition not having said plurality of nanoscale domains characterized by said nanofeatured surface geometry. The activity can include an enhancement of a biological property such as, for example, cell adhesion activity, cell proliferation activity, cell in-migration activity, cell differentiation activity, anti-bacterial activity, bactericidal activity, anti-inflammatory activity, osseointegration activity, osseoconduction activity, osseoinduction activity, reduction of immunoresponse, and combinations of these. In some embodiments of this aspect of the invention, the biological property is enhancement of cell adhesion activity, enhancement of cell proliferation activity, enhancement of anti-bacterial activity, and the enhancement of function or activity is equal to or greater than 100%. In another embodiment, the activity is an enhancement of a physical property such as surface hydrophilicity, surface free energy, surface hydrophobicity, sensing, drug transport, surface acidity, surface basicity, and combinations thereof. In one embodiment, the activity is increased hydrophilicity.

In an embodiment, the enhancement of cell adhesion activity or cell proliferation is equal to or greater than 10%, 20%, 30%, 40%, 50%, 60%, 70%, 80%, 90%, 100%, 120%, 140%, 160%, 180%, 200%, 250%, 300% or more.

In an embodiment, the said surface geometry is spatial distribution of relief features, recessed features, localized regions characterized by a selected composition, phase, crystallographic texture, or any combination of these, or a periodic or semi-periodic spatial distribution of said nanoscale domains. Alternatively, the surface geometry is a selected topology, topography, morphology, texture or any combination of these.

In one embodiment, each of said nanoscale domains are characterized by a vertical spatial dimension of between 50 nm and 1000 nm, between 150 nm and 500 nm, between 200 nm and 300 nm, or between 180 nm and 220 nm. Alternatively, the vertical spatial dimension may be between 220 nm and 250 nm, or between 250 nm and 290 nm, e.g. those dimensions achieved by an incident angle of a beam according to the present invention of about 0°, 45°, or 60°, respectively. The lateral spatial dimensions may be between 20 nm and 80 nm, or between 30 nm and 50 nm. Alternatively, the lateral spatial dimensions may be between 35 nm and 50 nm, or between 45 nm and 39 nm (for an incident angle of a beam according to the present invention of about 0°); may be between about 40 nm and about 30 nm, or between 39 nm and 30 nm (for an incident angle of a beam according to the present invention of about 45°); may be between about 30 nm and about 20 nm (for an incident angle of a beam according to the present invention of about 60°.) In another embodiment, the nanoscale domains comprise nanopillars or nanocolumns, or alternatively, nanoripples or any combination thereof having lateral spatial dimensions selected over the range of 10 to 100 nm and vertical spatial dimensions of 200 to 300 nm.

In an embodiment, the nanopillars or nanocolumns are inclined towards a direction oriented along a selected axis relative to said surface and/or are separated from one another by a distance of less than 100 nm.

The polymer composition may include wherein the polymer substrate is a fibrous protein substrate. In some embodiments, the fibrous protein substrate is a silk fibroin substrate, a collagen substrate, an elastin substrate, or a keratin substrate. In other embodiments, the polymer composition may include where the polymer substrate is a polysaccharide biopolymer substrate or a synthetic polymer substrate. In some embodiments, the polymer composition comprises a component of a medical device.

In an embodiment of the polymer composition, the directed energetic particle beam is a broad beam, focused beam, asymmetric beam, reactive beam or any combination of these, and wherein said one or more beam properties is intensity, fluence, energy, flux, incident angle, ion composition, neutral composition, ion to neutral ratio or any combinations thereof.

The present invention also includes a method of fabricating a polymer substrate composition providing enhanced activity. The method may include the steps of providing a polymer substrate, such as a fibrous protein substrate, having a substrate surface; and directing a directed energetic particle beam onto said substrate surface, thereby generating a plurality of nanoscale domains on said surface. In an embodiment, the directed energetic particle beam has one or more beam properties selected to generate said plurality of nanoscale domains characterized by a surface geometry providing enhanced cell adhesion or cell proliferation. In an embodiment, the directed energetic particle beam is a broad beam, focused beam asymmetric beam or any combination of these.

In an embodiment, the step of directing said directed energetic particle beam onto said substrate surface comprises directed irradiation synthesis (DIS), directed plasma nanosynthesis (DPNS), Direct Seeded Directed Plasma Nanosynthesis (DSDPNS), DSPNS (directed soft plasma nanosynthesis) or any combination of these. In an embodiment, the said one or more beam properties is intensity, fluence, energy, flux, incident angle, ion composition, neutral composition ion to neutral ratio or any combinations thereof. In an embodiment, the energetic particle beam comprises one or more ions, neutrals or combinations thereof. In some embodiments, wherein said ions are krypton ions, argon ions, oxygen. In an embodiment, one or more beam properties comprise incident angle and said incident angle is selected from the range of 0° to 80°. In some embodiments, the incident angle is 0°, 45° or 60°. In embodiments, the one or more beam properties comprise fluence and said fluence is selected from the range of 1×10¹⁶ cm⁻² to 1×10¹⁹ cm⁻². In embodiments, the one or more beam properties comprise energy and said energy is selected from the range of 0.01 eV to 10 keV.

In an embodiment, the polymer composition or polymer substrate composition, fabricated by methods of the invention, will retain its surface geometry providing the selected function, even after a change of conditions. The change of conditions may include a change in the environmental conditions of the fabricated materials, such as, without limitation, change from air immersion to liquid immersion, change in temperature conditions, change in liquid immersion conditions such as change in pH, or change in excipient concentrations such as change in salt concentrations. Liquid immersion may include aqueous or non-aqueous conditions, such as a change in ionic strength or ionic composition of a fluid, such as, for example, a biofluid. For example, Applicant has demonstrated that an argon-treated bacterial nanocellulose material of the present invention, after fabrication, can be immersed in aqueous solution and so-treated material retains its surface geometry, e.g., nanopillar structure, and demonstrates anti-bacterial activity, e.g., appearance of E. coli as flat with damaged and broken bodies typical of a dead cell, see FIG. 85(B). Applicant has demonstrated that the hydrogels irradiated by methods of the invention retain their nanofeatures even in liquid media, after contact with media. The nanofeatures are stable in air or in hydrophilic media, and retain their functionality, e.g., ability to kill bacteria. Methods and compositions of the present invention exhibit a beneficial physical stability of a nanostructure, for example, maintaining physical dimensions and mechanical properties of nanofeatures during exposure to a fluid such as a biofluid. Methods and compositions of the present invention exhibit a beneficial stability with respect to time, with respect to enhanced aging attribute wherein the surface geometries and the selected functions are maintained for a useful duration of time when provided to an environment, such as an in vitro or in vivo environment, for a period of time, such as, for example, one hour or more, one day or more, one month or more.

### BRIEF DESCRIPTION OF THE DRAWINGS

FIG. 1. 600 eV Ar⁺ irradiation of polycrystalline Zn (top) and 6 keV O₂⁺ irradiation of Zn forming ZnO tips (bottom).

FIG. 2. Diagram of dual ion beam experiment device.

FIG. 3. Diagram (cartoon) showing codeposition experimental setup.

FIG. 4. Simulated ion collisions of 500 eV Ar⁺ on Zn at 45° as seen from within the target (left) and from above (right).

FIG. 5. Shows the energy distribution of Zn surface atoms impacted by the 500 eV Ar⁺ beam.

FIG. 6. Simulated ion collisions of 1000 eV Ar⁺ on Zn at 45° as seen from within the target (left, top) and from above (left, bottom). Right, energy distribution of Zn atoms sputtered by 100 eV Ar⁺ at 45°.

FIG. 7. Simulated ion collisions of 500 eV O₂⁺ on Zn/SiO₂ at 0° as seen from within the target (left, top) and from above (left, bottom). Simulated ion collisions of 1000 eV O₂⁺ on Zn/SiO₂ at 0° as seen from within the target (right, top) and from above (right, bottom).

FIG. 8. AFM scans of Zn codeposition on Si, deposition with 500 eV Ar⁺, sample irradiated with 500 eV Ar⁺ to 1E17 ions/cm² with flux ratios of 0.1 (top; left, right), 0.2 (second from top, left, right), 0.5 (third from top, left, right), 1.0 (second from bottom, left, right), and 2.0 (bottom, left, right.)

FIG. 9. RMS Roughness over Flux Ratios for 500 eV Ar⁺ Codeposition on Si.

FIG. 10. AFM scans of Zn codeposition on Si, deposition with 1000 eV Ar⁺, sample irradiated with 1000 eV Ar⁺ to 1E17 ions/cm2 with flux ratios of 0.1 (top, left, right), 0.2 (second from top, left, right), 0.5 (third from top, left, right), 1.0 (second from bottom, left, right), and 2.0 (bottom, left, right.)

FIG. 11. RMS Roughness over Flux Ratios for 1000 eV Ar⁺ Codeposition on Si.

FIG. 12. AFM scans of Zn codeposition on Si, deposition with 500 eV Ar⁺, sample irradiated with 500 eV O₂⁺ to 1E17 ions/cm² with flux ratios of 0.1 (top, left, right), 0.2 (second from top, left, right), 0.5 (third from top, left, right), 1.0 (second from bottom, left, right), and 2.0 (bottom, left, right).

FIG. 13. RMS roughness over Flux ratios for 500 eV O₂⁺ codeposition on Si.

FIG. 14. AFM scans of Zn codeposition on Si, deposition with 1000 eV Ar⁺, sample irradiated with 1000 eV O₂⁺ to 1 E17 ions/cm² with flux ratios of 0.1 (top, left, right), 0.2 (second from top, left, right), 0.5 (third from top, left, right), 1.0 (second from bottom, left, right), and 2.0 (bottom, left, right).

FIG. 15. RMS Roughness over Flux Ratios for 1000 eV O₂⁺ Codeposition on Si.

FIG. 16. AFM of Codeposition on Si surfaces with Ar⁺ irradiation with top left 500 eV, 0.1 flux ratio, top right 1000 eV, 0.1 flux ratio, middle left 500 eV, 0.5 flux ratio, middle right 1000 eV, 0.5 flux ratio, bottom left 500 eV, 2.0 flux ratio, bottom right 1000 eV, 2.0 flux ratio.

FIG. 17. AFM of Codeposition on Si surfaces with O₂⁺ irradiation with top left 500 eV, 0.5 flux ratio, top right 1000 eV, 0.5 flux ratio, bottom left 500 eV, 1.0 flux ratio, and bottom right 1000 eV, 1.0 flux ratio.

FIG. 18. AFM scans of Zn codeposition on Si, deposition with 500 eV O₂⁺, sample irradiated with 500 eV O₂⁺ to 1 E17 ions/cm² (top left, right) and 5E17 ions/cm² (bottom left, right) with a flux ratio of 1.0.

FIG. 19. AFM scans of Zn codeposition on Si, deposition with 1000 eV O₂⁺, sample irradiated with 1000 eV O₂⁺ to 1 E17 ions/cm² (top, left, right) and 5E17 ions/cm² (bottom, left, right) with a flux ratio of 1.0

FIG. 20A. RMS Roughness of Zn Codeposition on Si Highlighting Fluence and Energy Effects.

FIG. 20B. Feature Density of Zn Codeposition on Si Highlighting Fluence and Energy Effects.

FIG. 20C. Feature Density on Si from Zn Codeposition.

FIG. 20D. Surface Roughness from AFM of Si from Zn Codeposition.

FIG. 21. Virgin PDMS AFM height scans.

FIG. 22. AFM height analysis of PDMS irradiated at normal incidence with 500 eV Ar⁺ to 1E17 ions/cm².

FIG. 23. AFM height analysis of PDMS irradiated at normal incidence with 1000 eV Ar⁺ to 1E17 ions/cm².

FIG. 24A-J AFM height scans of Zn codeposition on PDMS, deposition with 500 eV Ar⁺, sample irradiated with 500 eV Ar⁺ to 1E17 ions/cm² with flux ratios of 0.1 (24A, 24B), 0.2 (24C, 24D), 0.5 (24E, 24F), 1.0 (24G, 24H), and 2.0 (24I, 24J).

FIG. 25A-E. AFM amplitude scans of Zn codeposition on PDMS, deposition with 500 eV Ar⁺, sample irradiated with 500 eV Ar⁺ to 1E17 ions/cm² with flux ratios of 0.1 (25A), 0.2 (25B), 0.5 (25C), 1.0 (25D), and 2.0 (25E).

FIG. 26. Surface Roughness from AFM of PDMS from Zn Codeposition with 500 eV Ar⁺.

FIG. 27A-J. AFM height scans of Zn codeposition on PDMS, deposition with 1000 eV Ar⁺, samples irradiated with 1000 eV Ar⁺ to 1E17 ions/cm² with flux ratios of 0.1 (A, B), 0.2 (C, D), 0.5 (E, F), 1.0 (G, H), and 2.0 (I, J).

FIG. 28A-C. AFM amplitude scans of Zn codeposition on PDMS, deposition with 1000 eV Ar⁺, samples irradiated with 1000 eV Ar⁺ to 1E17 ions/cm² with flux ratios of 0.2 (A), 1.0 (B), and 2.0 (C).

FIG. 29. Surface Roughness from AFM of PDMS from Zn Codeposition with 1000 eV Ar⁺.

FIG. 30A-J.: AFM height scans of Zn codeposition on PDMS, deposition with 500 eV Ar⁺, samples irradiated with 500 eV O₂⁺ to 1E17 ions/cm² with flux ratios of 0.1 (30A, 30B), 0.2 (30C, 30D), 0.5 (30E, 30F), 1.0 (30G, 30H), and 2.0 (30I, 30J).

FIG. 31A-C. AFM amplitude scans of Zn codeposition on PDMS, deposition with 500 eV Ar⁺, samples irradiated with 500 eV O₂⁺ to 1E17 ions/cm² with flux ratios of 0.2 (31A), 0.5 (31B), 1.0 (31C).

FIG. 32. Surface Roughness from AFM of PDMS from Zn Codeposition with 500 eV O₂.

FIG. 33A-J. AFM height scans of Zn codeposition on PDMS, deposition with 1000 eV Ar⁺, sample irradiated with 1000 eV O₂⁺ to 1E17 ions/cm² with flux ratios of 0.1 (33A, 33B), 0.2 (33C, 33D), 0.5 (33E, 33F), 1.0 (33G, 33H), and 2.0 (33I, 33J).

FIG. 34A-B. AFM amplitude scans of Zn codeposition on PDMS, deposition with 1000 eV Ar⁺, sample irradiated with 1000 eV O₂⁺ to 1E17 ions/cm² with flux ratios of 1.0 (34A), and 2.0 (34B).

FIG. 35. Surface Roughness from AFM of PDMS from Zn Codeposition with 1000 eV O₂⁺.

FIG. 36A-F. AFM height of Codeposition on PDMS surfaces with Ar⁺ irradiation with 36A) 500 eV, 0.1 flux ratio, 36B) 1000 eV, 0.1 flux ratio, 36C) 500 eV, 1.0 flux ratio, 36D) 1000 eV, 1.0 flux ratio, 36E) 500 eV, 2.0 flux ratio, 36F) 1000 eV, 2.0 flux ratio.

FIG. 37A-E. AFM amplitude of Codeposition on PDMS surfaces with Ar⁺ irradiation with 37A) 500 eV, 0.1 flux ratio, 37B) 1000 eV, 0.1 flux ratio, 37C) 500 eV, 1.0 flux ratio, 37D) 1000 eV, 1.0 flux ratio, 37E) 500 eV, 2.0 flux ratio, 37F) 1000 eV, 2.0 flux ratio.

FIG. 38A-F. AFM height of Codeposition on PDMS surfaces with O₂⁺ irradiation with 38A) 500 eV, 0.1 flux ratio, 38B) 1000 eV, 0.1 flux ratio, 38C) 500 eV, 0.2 flux ratio, 38D) 1000 eV, 0.2 flux ratio, 38E) 500 eV, 1.0 flux ratio, 38F) 1000 eV, 1.0 flux ratio.

FIG. 39A-D. AFM amplitude of Codeposition on PDMS surfaces with O₂⁺ irradiation with 39A) 500 eV, 0.2 flux ratio, 39B) 1000 eV, 0.2 flux ratio, 39C) 500 eV, 1.0 flux ratio, 39D) 1000 eV, 1.0 flux ratio.

FIG. 40A-D. AFM height scans of Zn codeposition on PDMS, deposition with 500 eV Ar⁺, samples irradiated with 500 eV O₂⁺ to 1E17 ions/cm² (40A, 40B) and 5E17 ions/cm² (40C, 40D) with a flux ratio of 1.0.

FIG. 41A-B. AFM amplitude scans of Zn codeposition on PDMS, deposition with 500 eV Ar⁺, samples irradiated with 500 eV O₂⁺ to 1E17 ions/cm² (41A) and 5E17 ions/cm² (41B) with a flux ratio of 1.0.

FIG. 42A-D. AFM height scans of Zn codeposition on PDMS, deposition with 1000 eV Ar⁺, samples irradiated with 1000 eV O₂⁺ to 1E17 ions/cm² (42A, 42B) and 5E17 ions/cm² (42C, 42D) with a flux ratio of 1.0.

FIG. 43A-C. AFM amplitude scans of Zn codeposition on PDMS, deposition with 1000 eV Ar⁺, samples irradiated with 1000 eV O₂⁺ to 1E17 ions/cm² (43A) and 5E17 ions/cm² (43B, 43C) with a flux ratio of 1.0.

FIG. 44. Survey Scan of Si after Codeposition with 500 eV O₂⁺, a final fluence of 5E17 ions/cm², and flux ratio of 1.0.

FIG. 45. Region Scan of Zn Peaks on Si after Codeposition with 500 eV O₂⁺, a final fluence of 5E17 ions/cm², and flux ratio of 1.0.

FIG. 46. Atomic Concentration of Zn on Si after Codeposition Organized by Flux Ratio.

FIG. 47. Atomic Concentration of 0 on Si after Codeposition Organized by Flux Ratio.

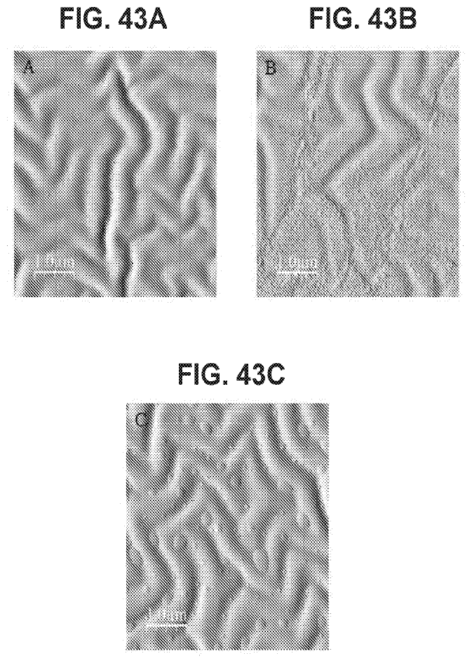

FIG. 43A-C. AFM amplitude scans of Zn codeposition on PDMS, deposition with 1000 eV Ar⁺, samples irradiated with 1000 eV O₂⁺ to 1E17 ions/cm² (43A) and 5E17 ions/cm² (43B, 43C) with a flux ratio of 1.0.

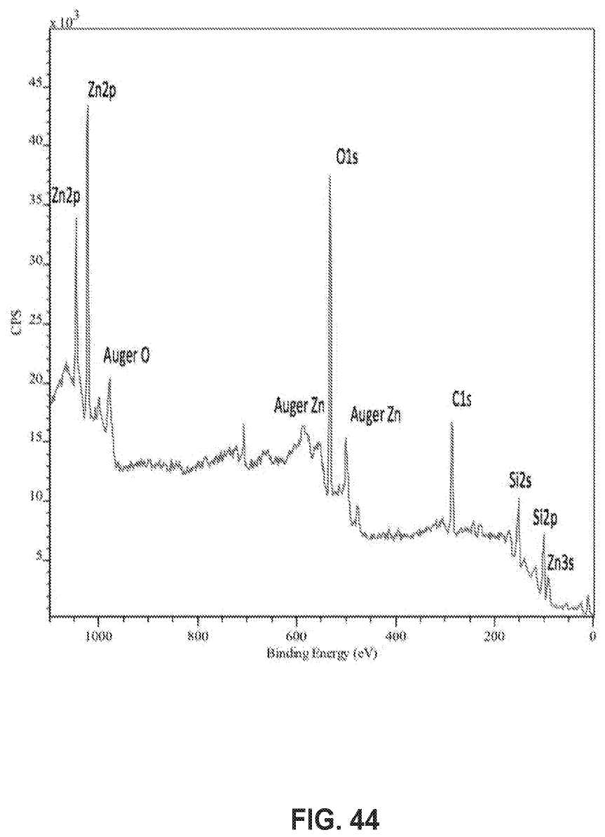

FIG. 44. Survey Scan of Si after Codeposition with 500 eV O₂⁺, a final fluence of 5E17 ions/cm², and flux ratio of 1.0.

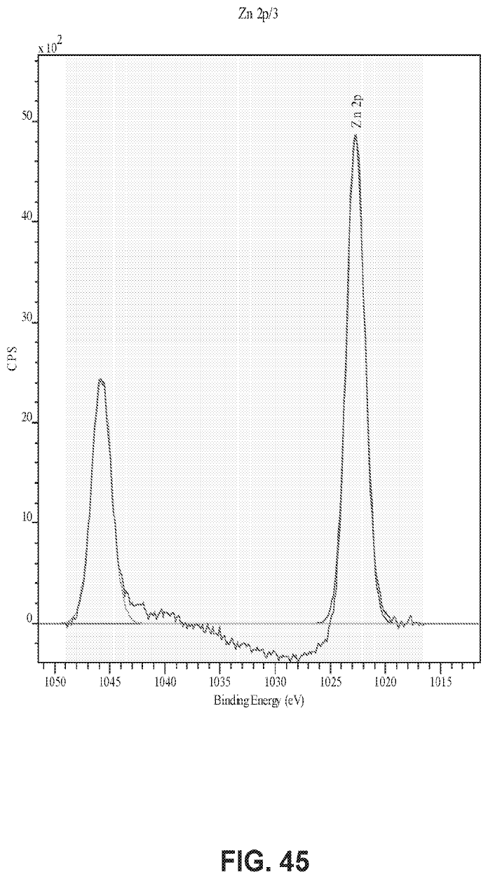

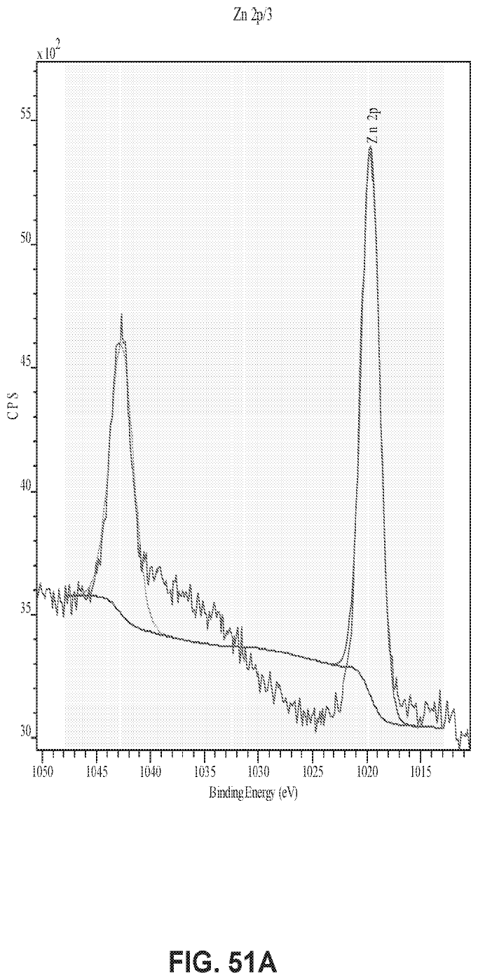

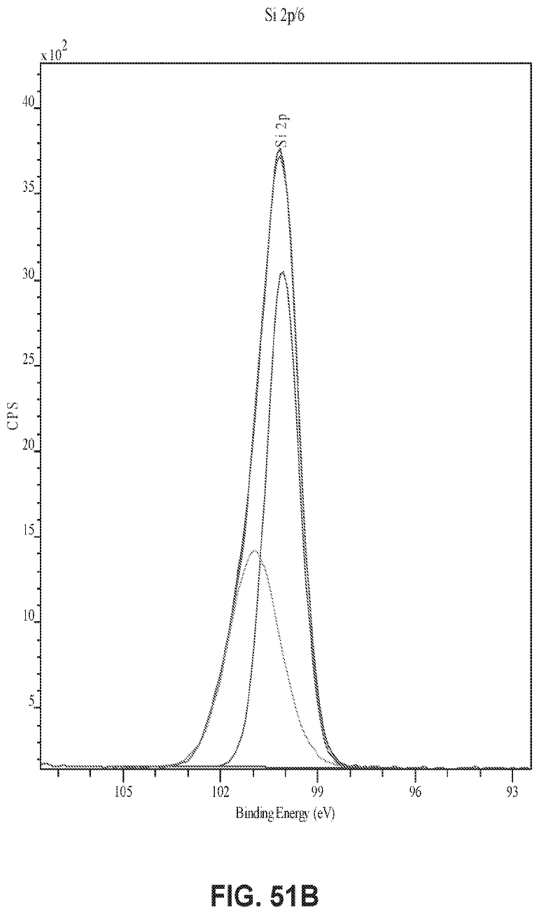

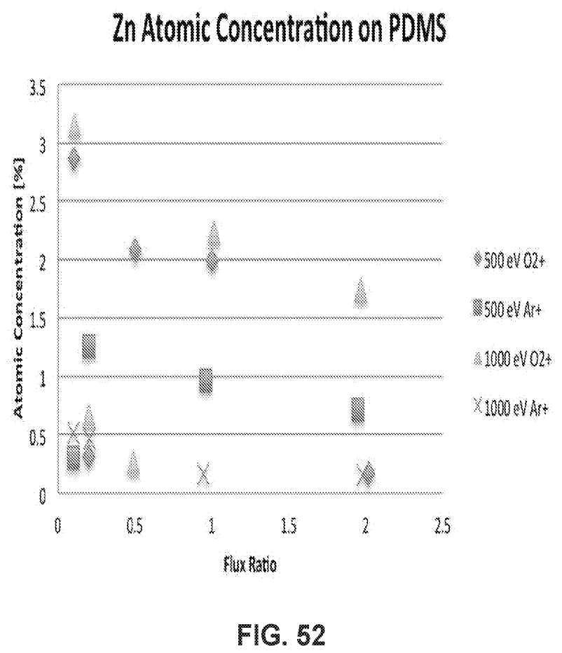

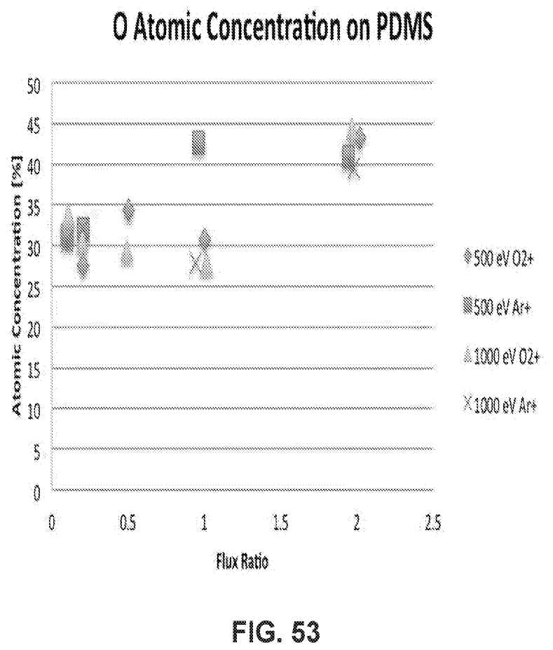

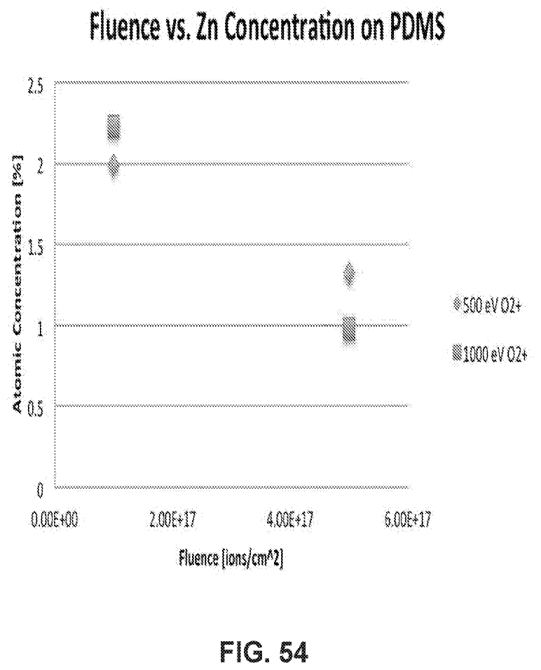

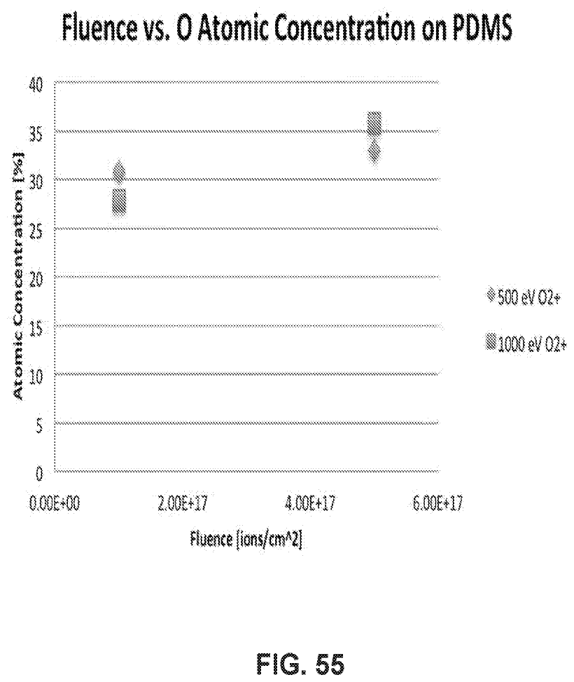

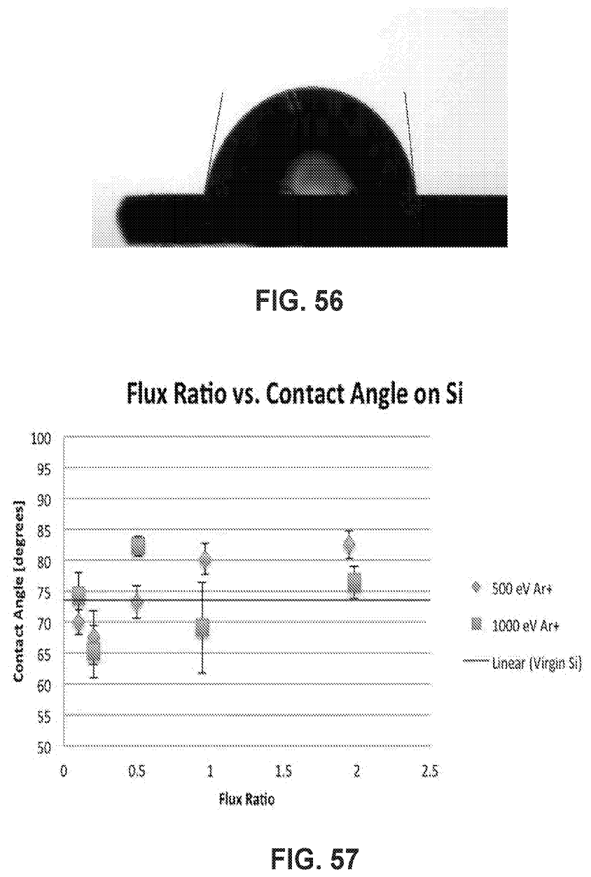

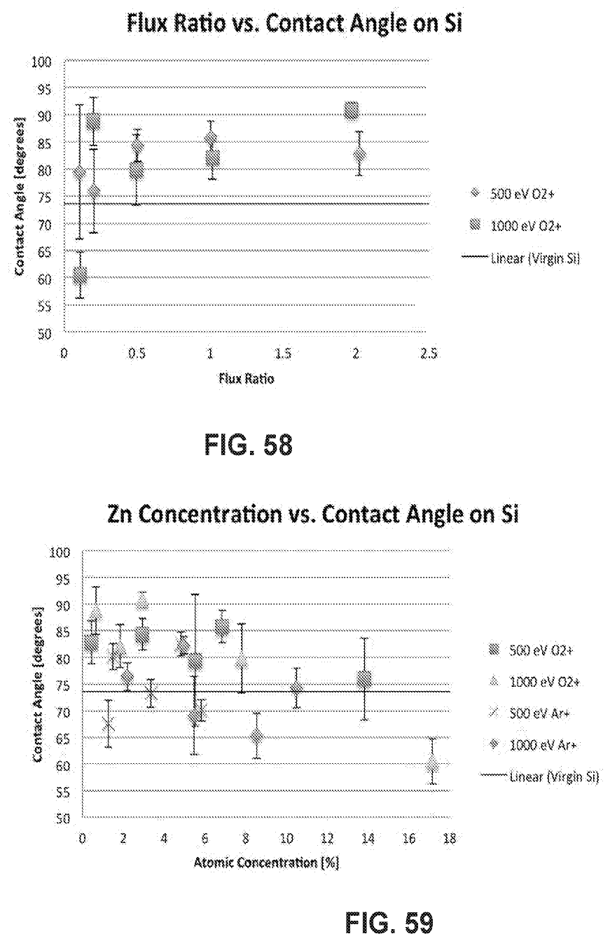

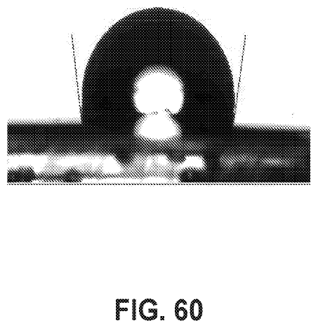

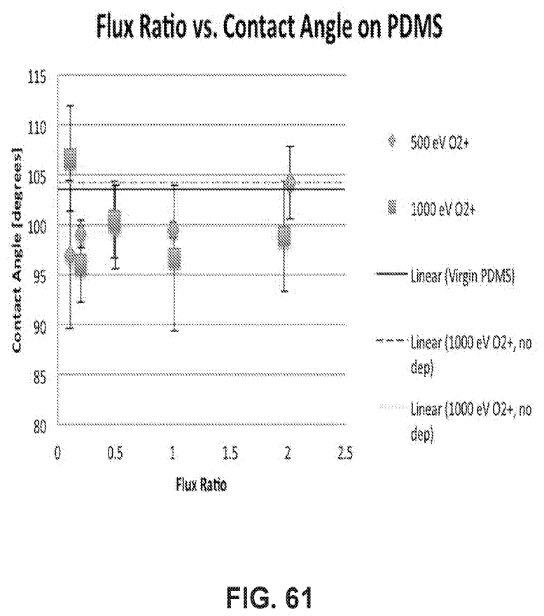

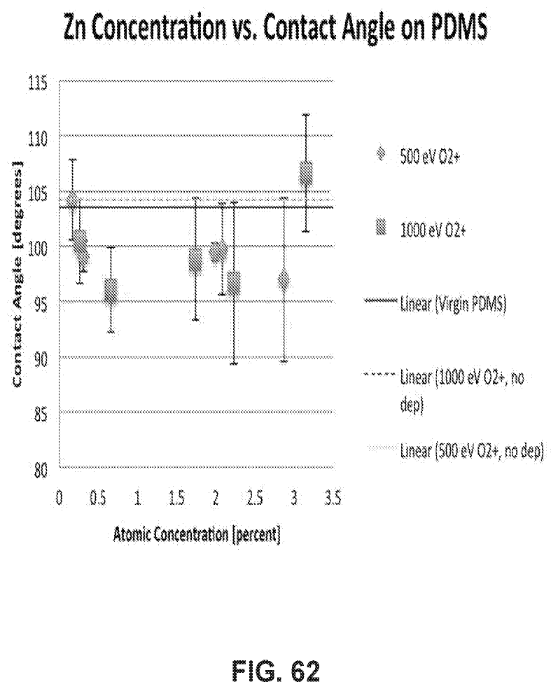

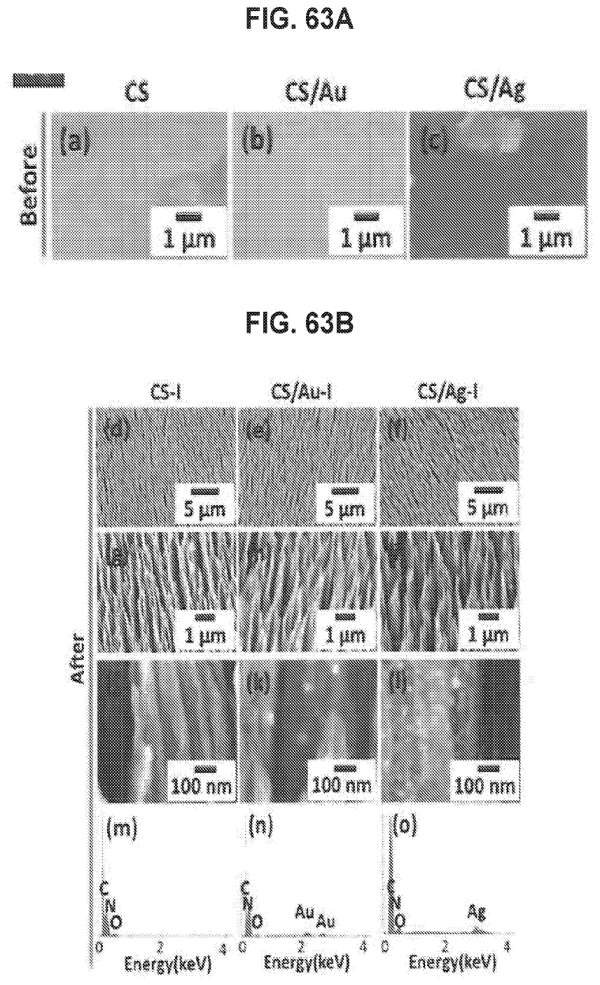

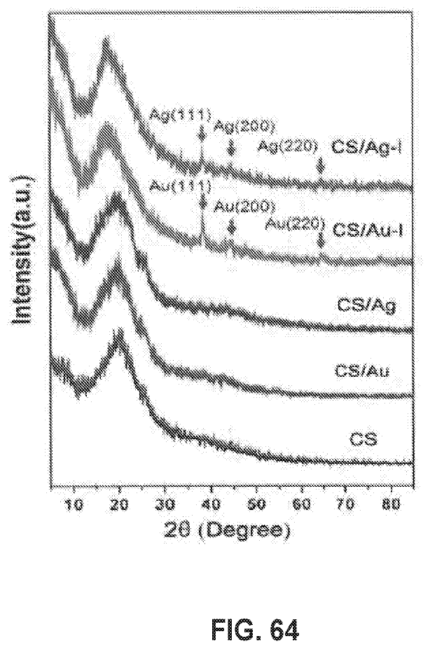

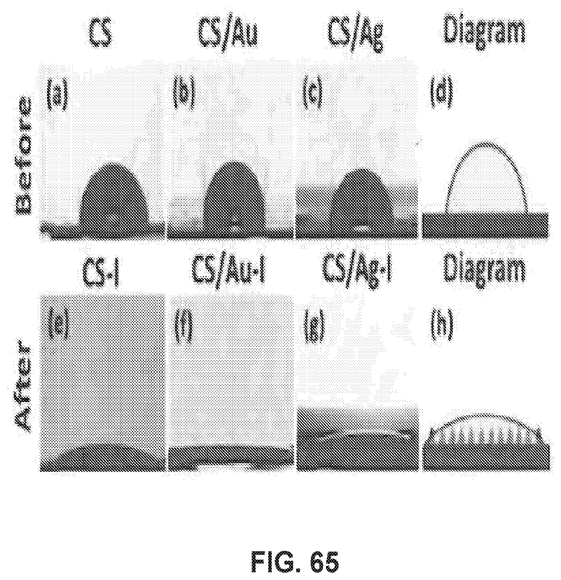

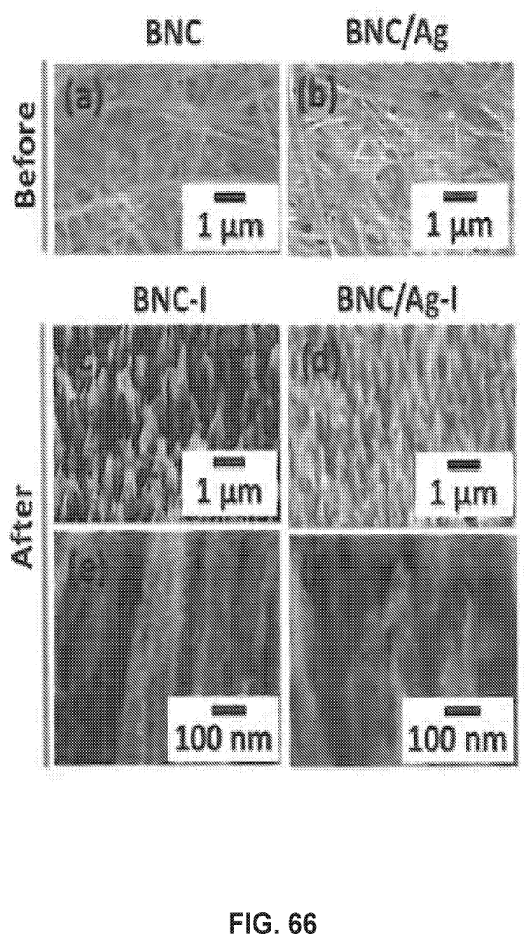

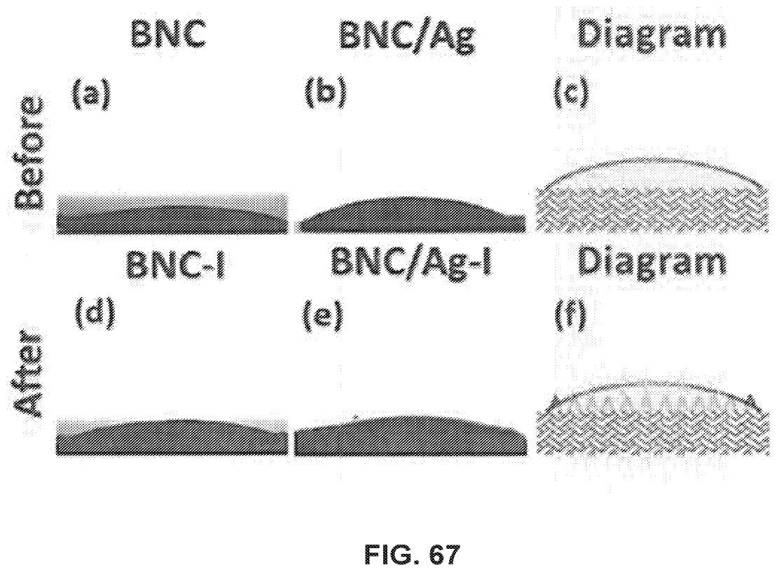

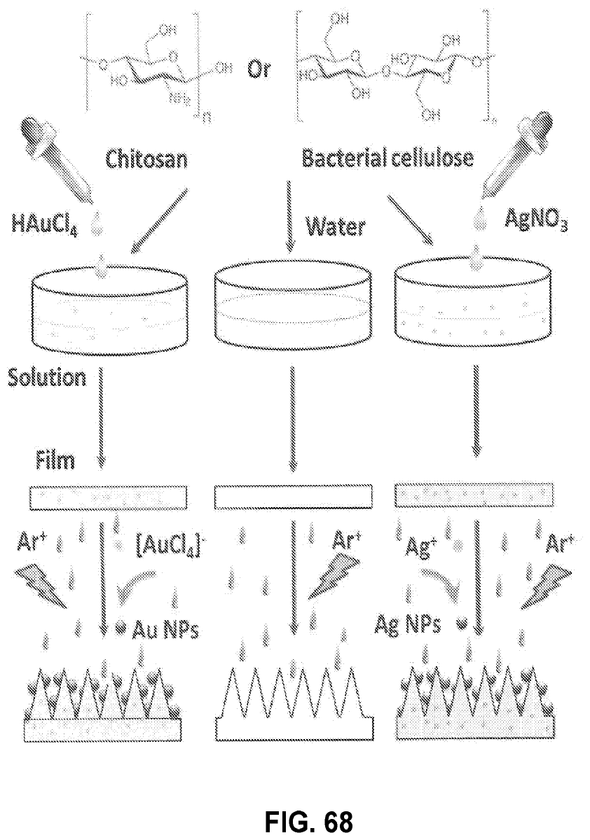

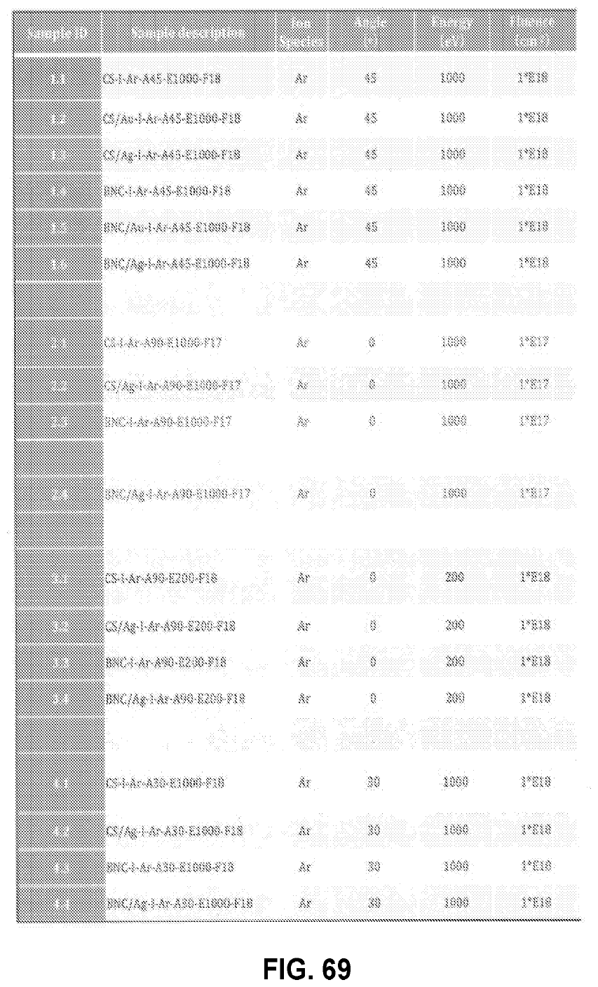

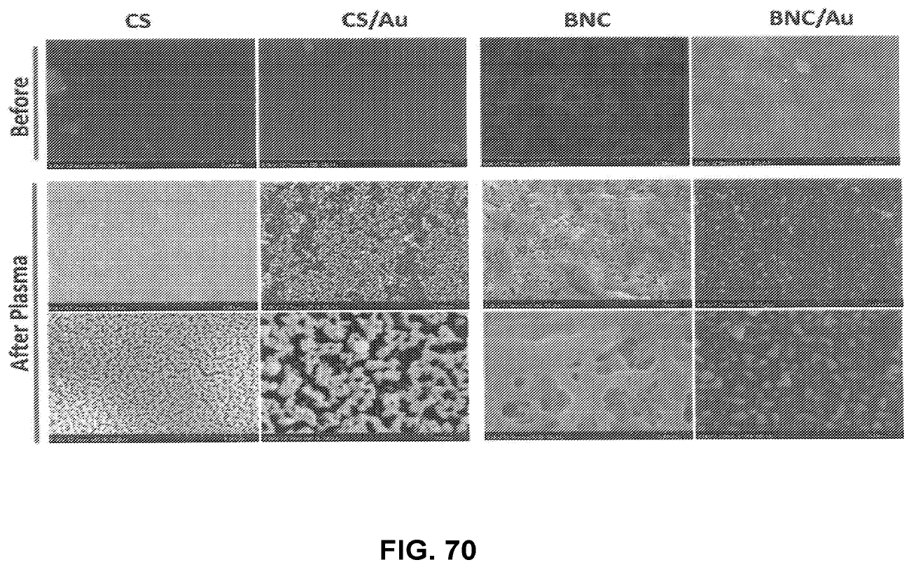

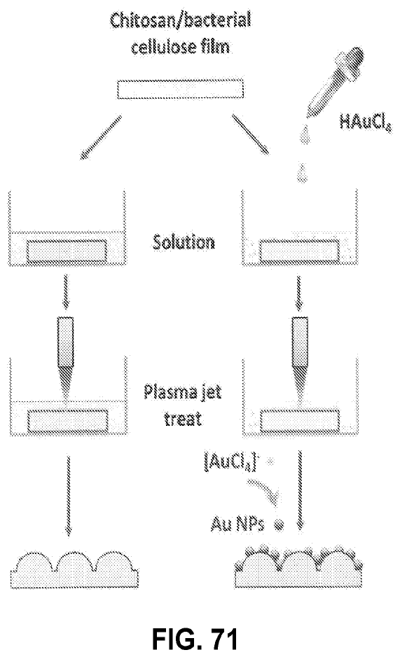

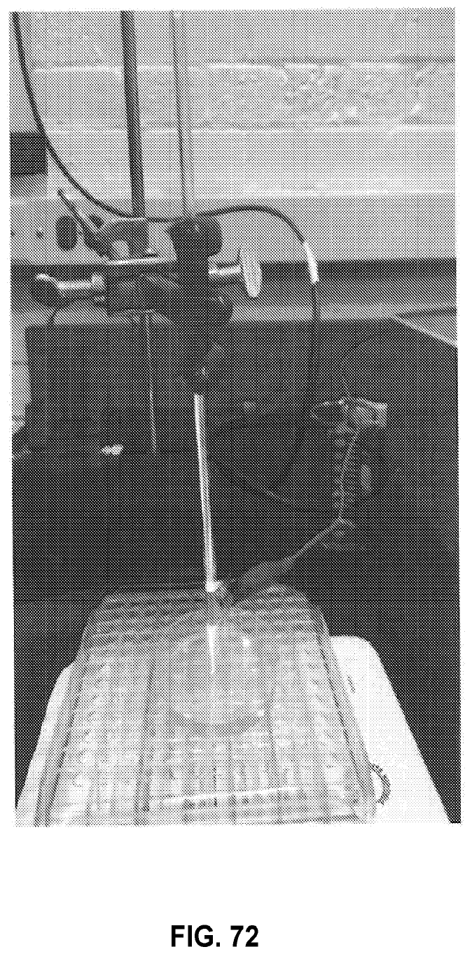

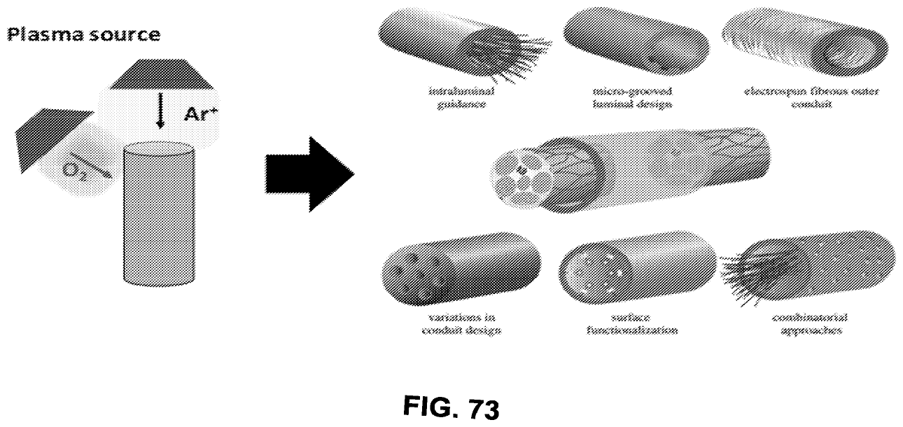

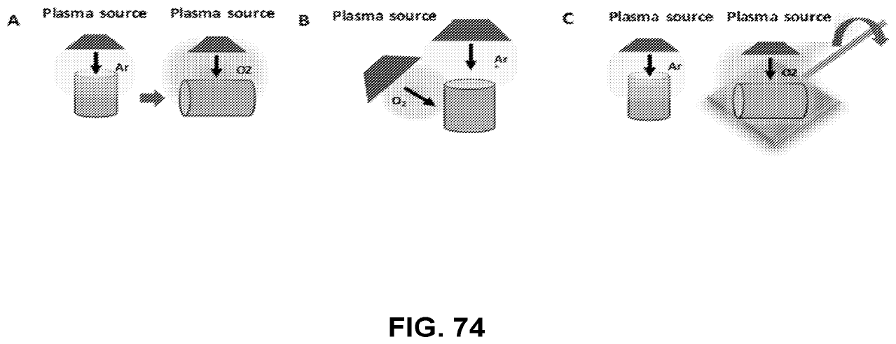

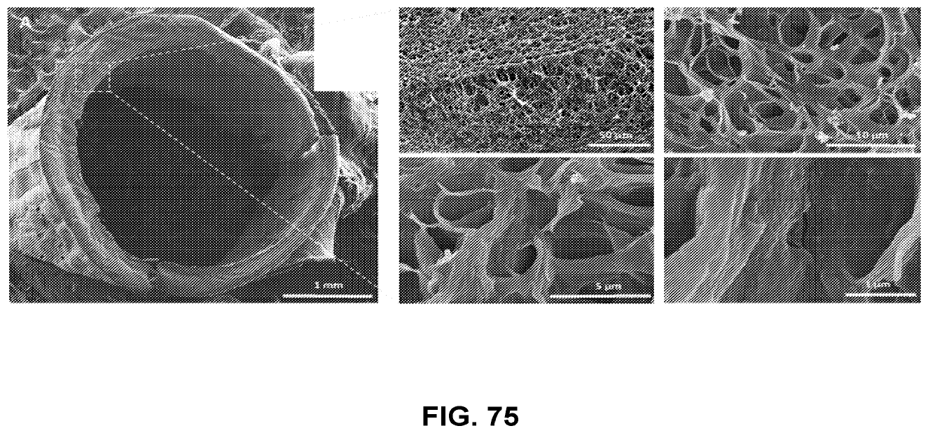

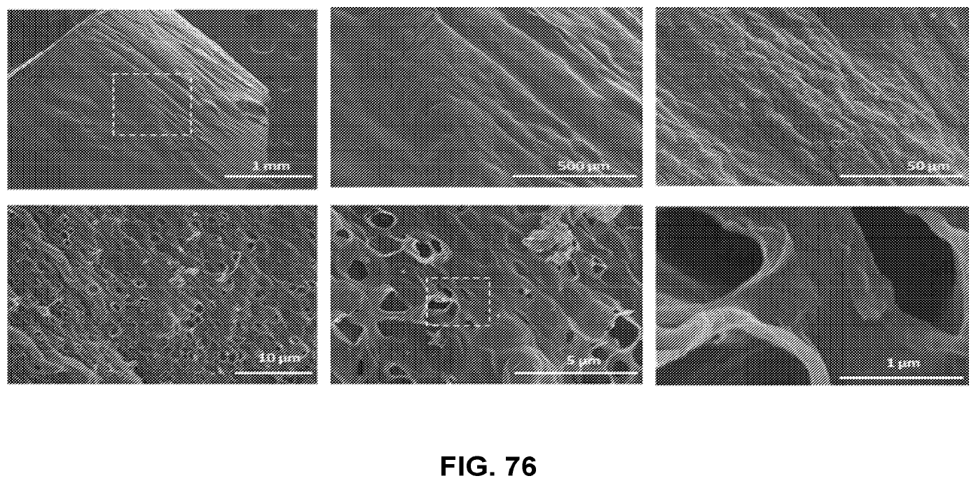

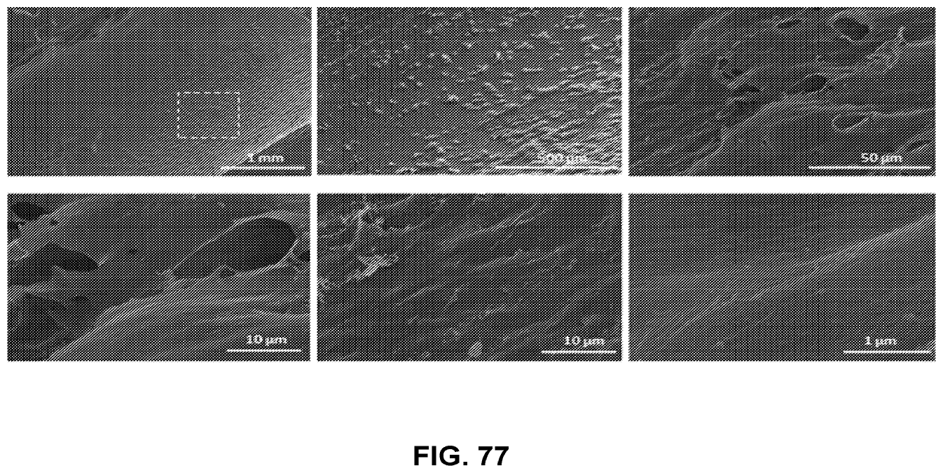

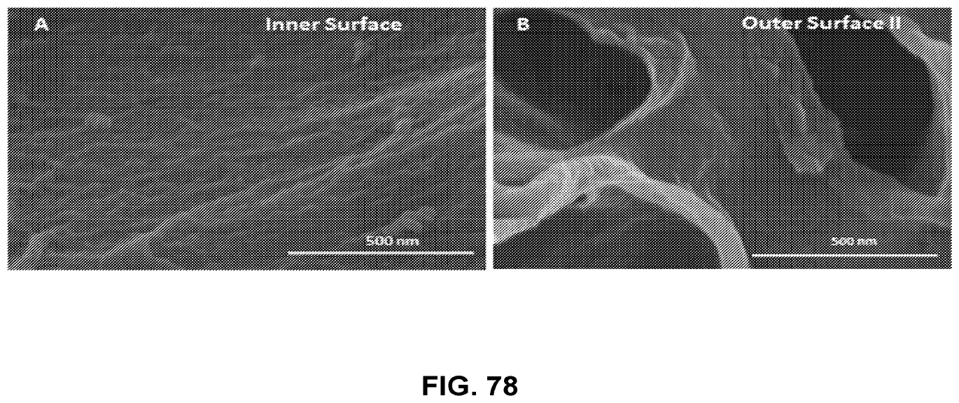

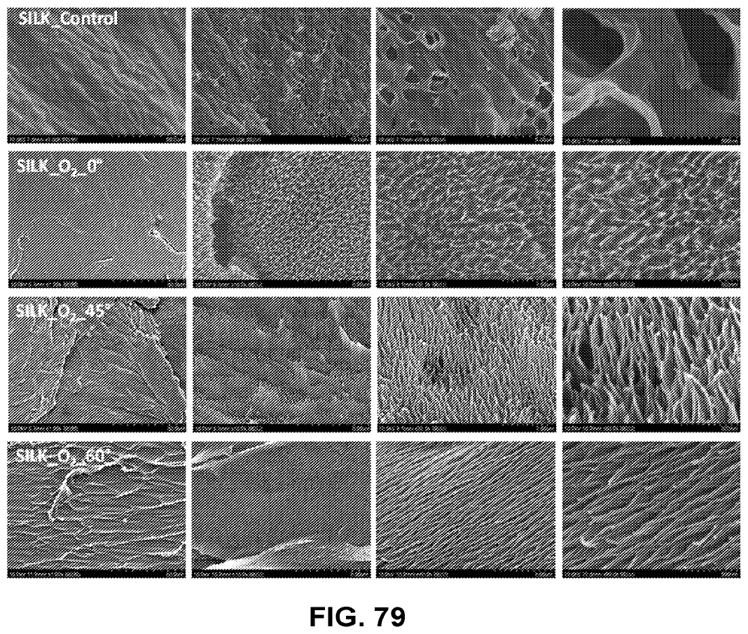

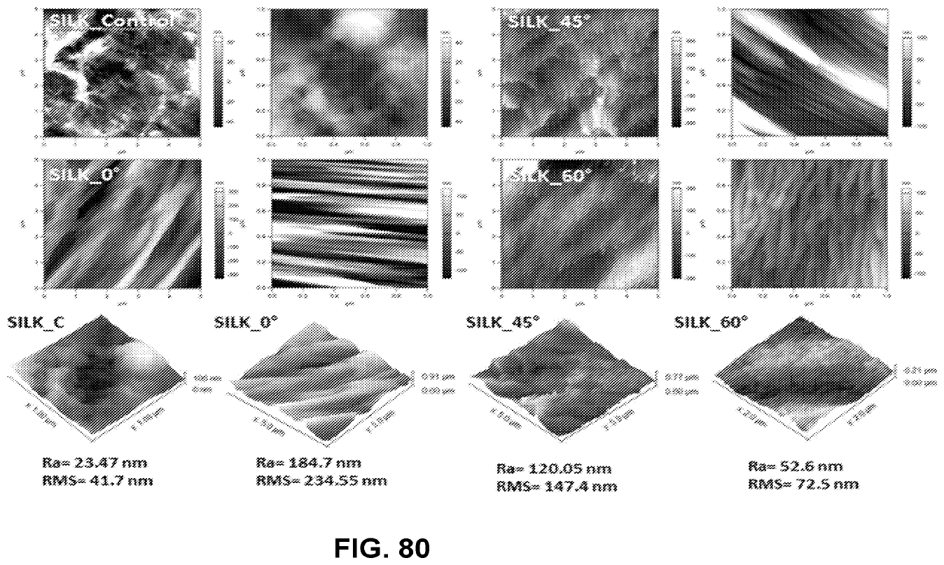

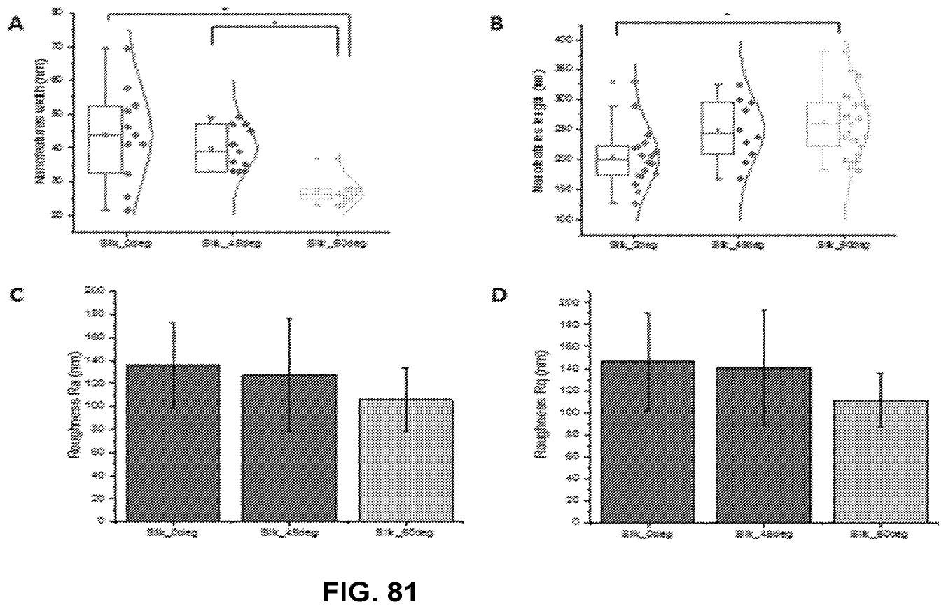

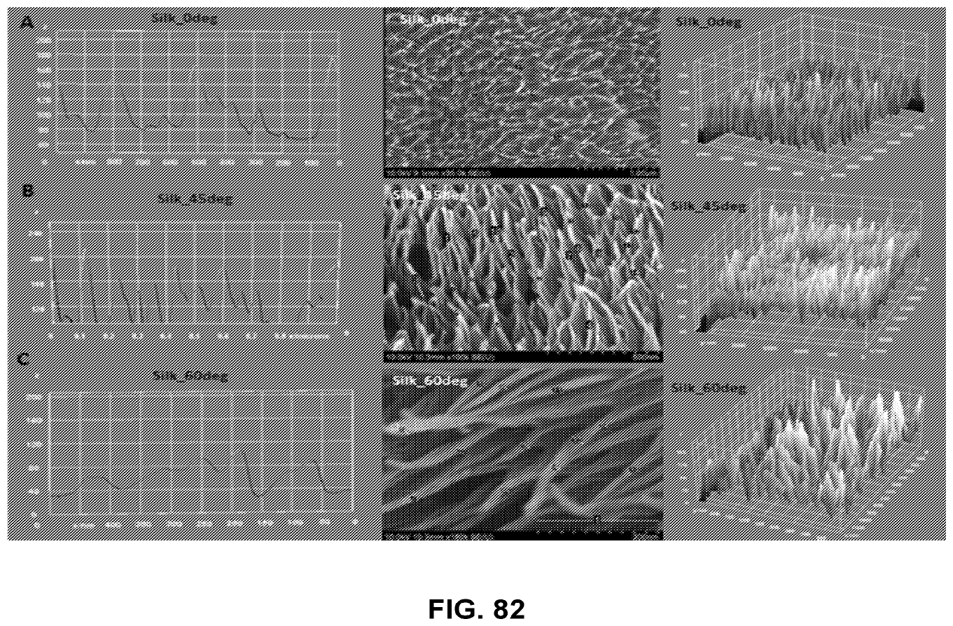

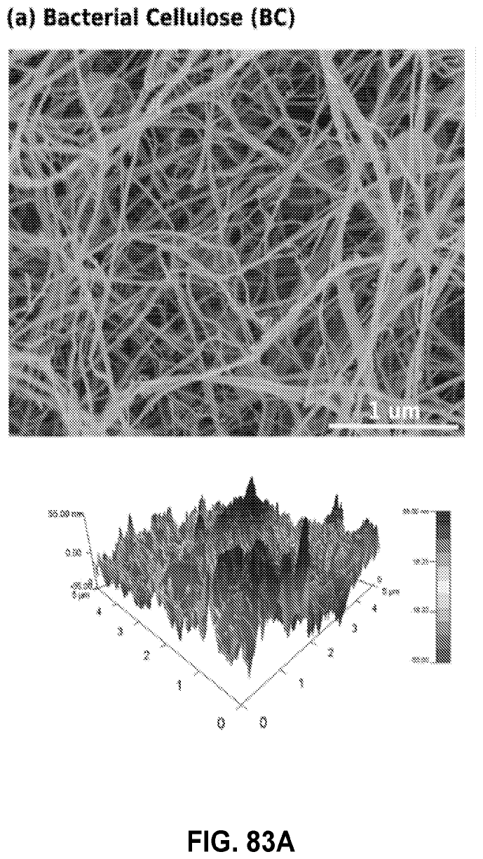

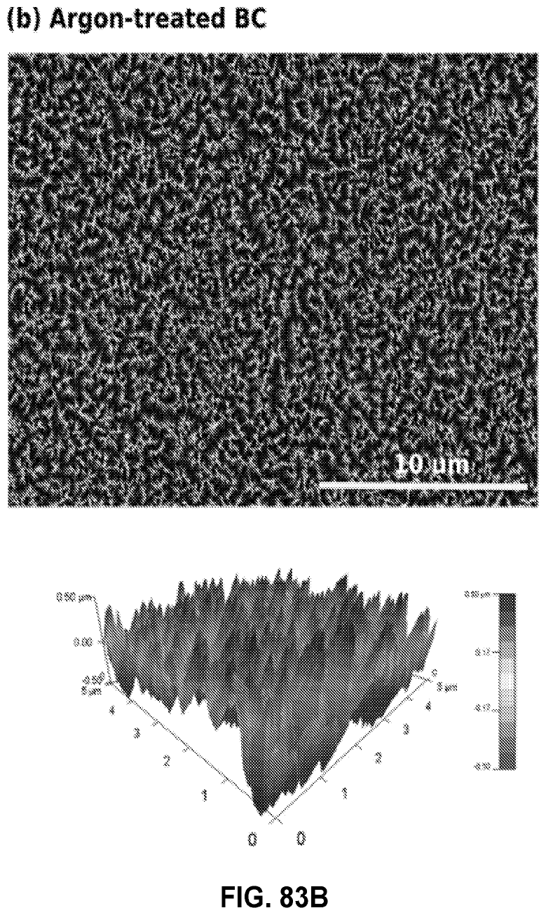

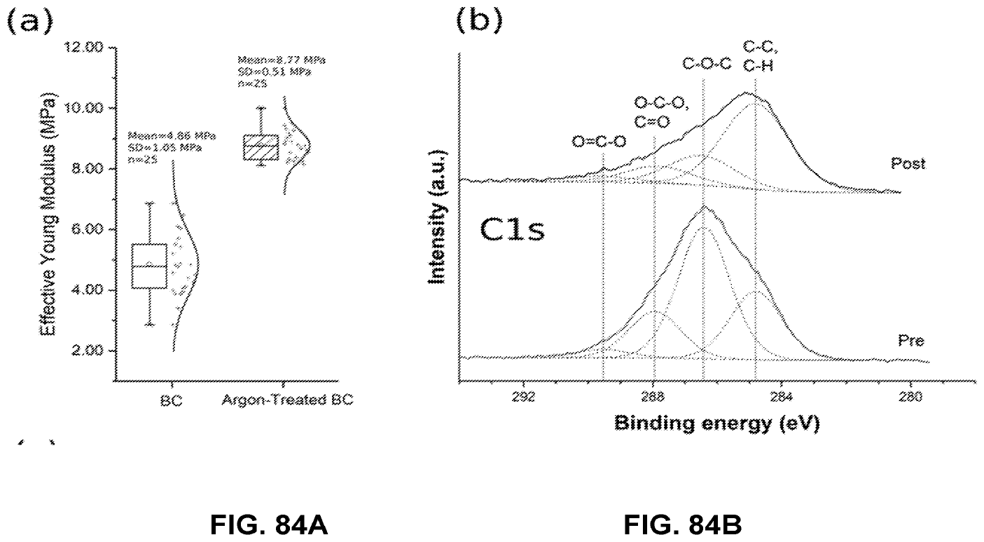

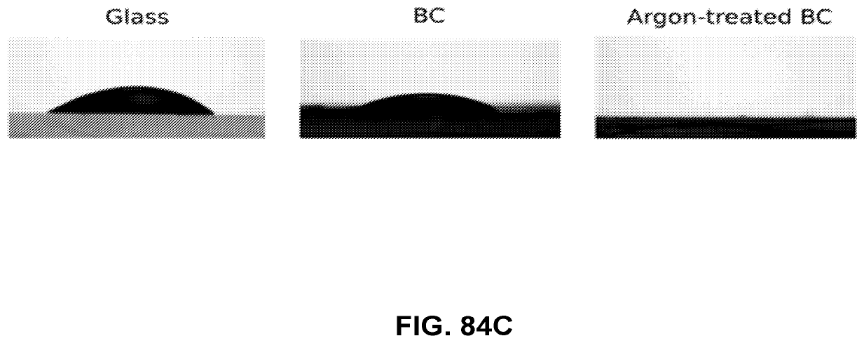

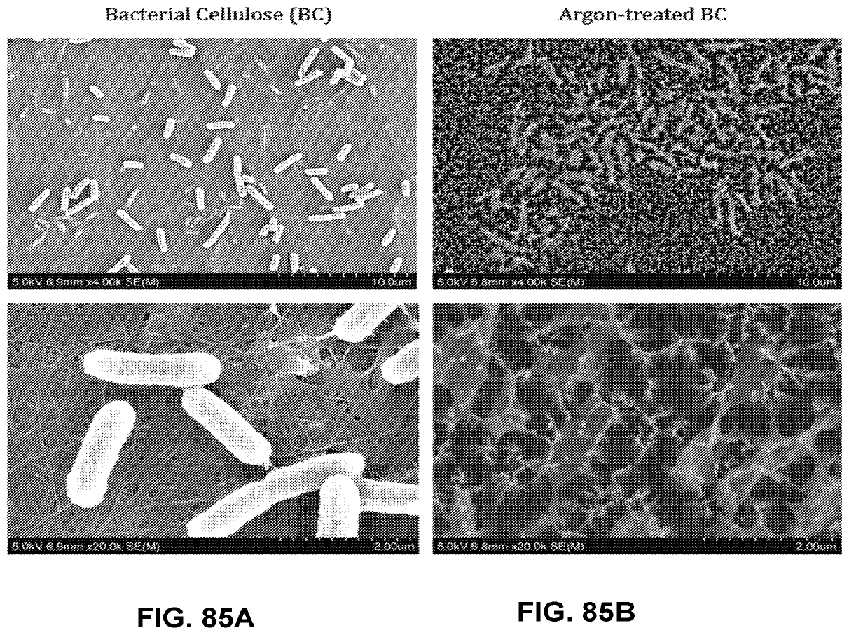

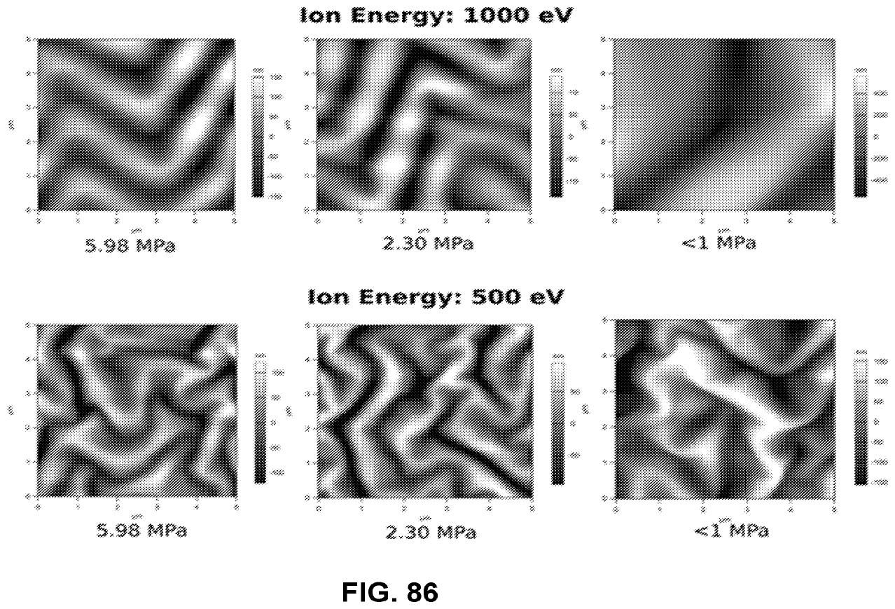

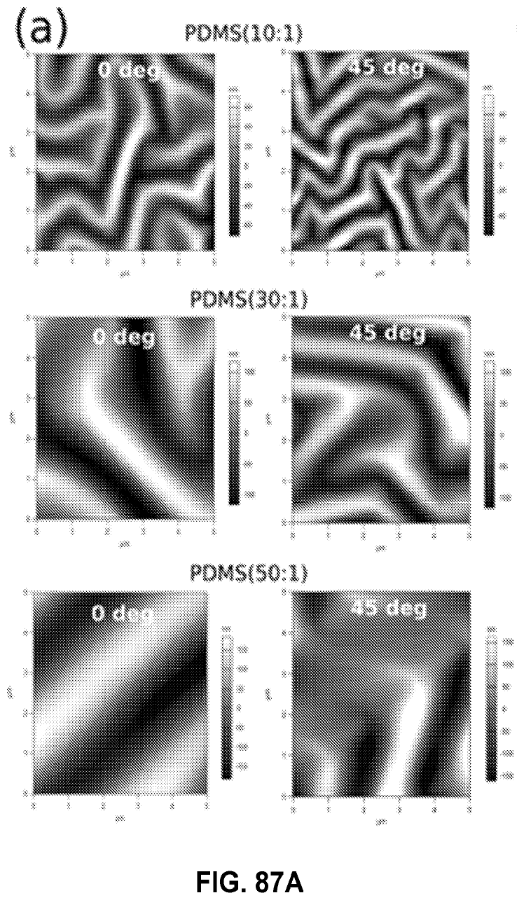

FIG. 45. Region Scan of Zn Peaks on Si after Codeposition with 500 eV O₂⁺, a final fluence of 5E17 ions/cm², and flux ratio of 1.0.

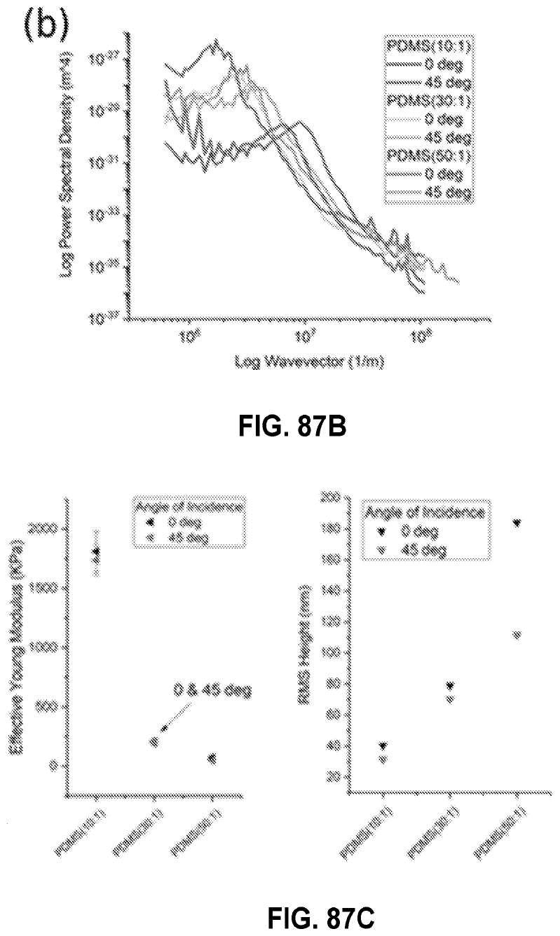

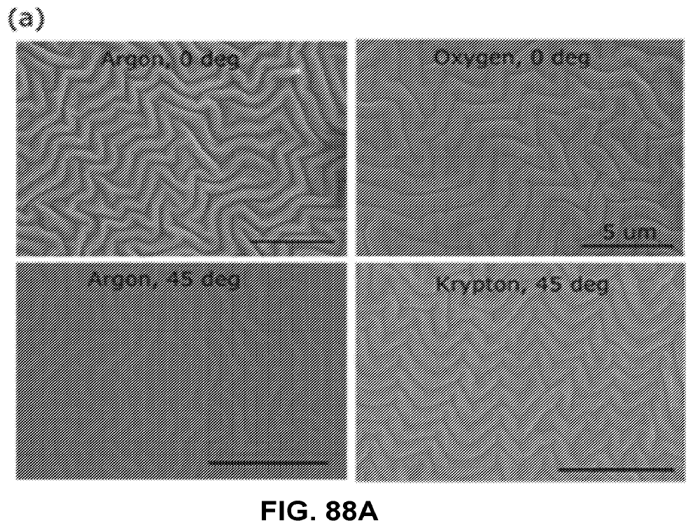

FIG. 46. Atomic Concentration of Zn on Si after Codeposition Organized by Flux Ratio.

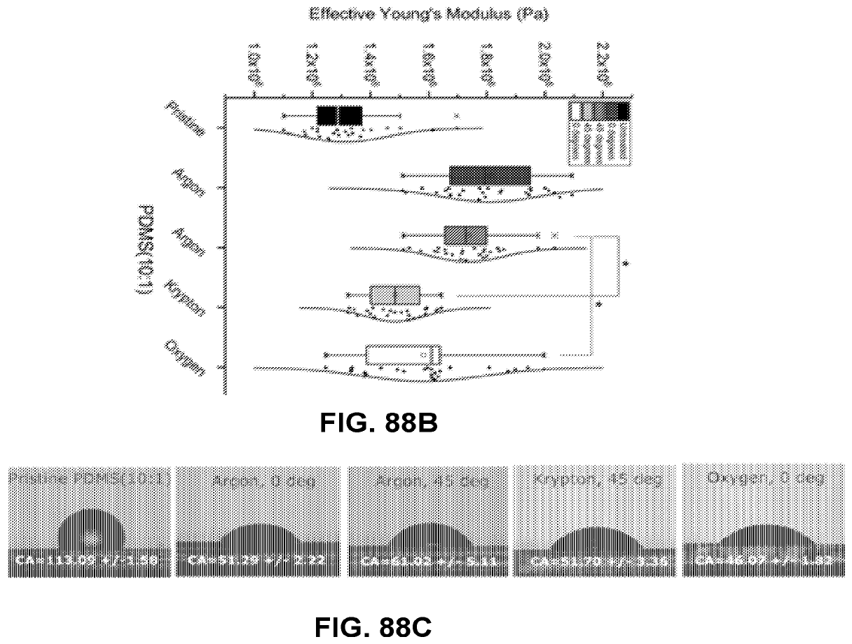

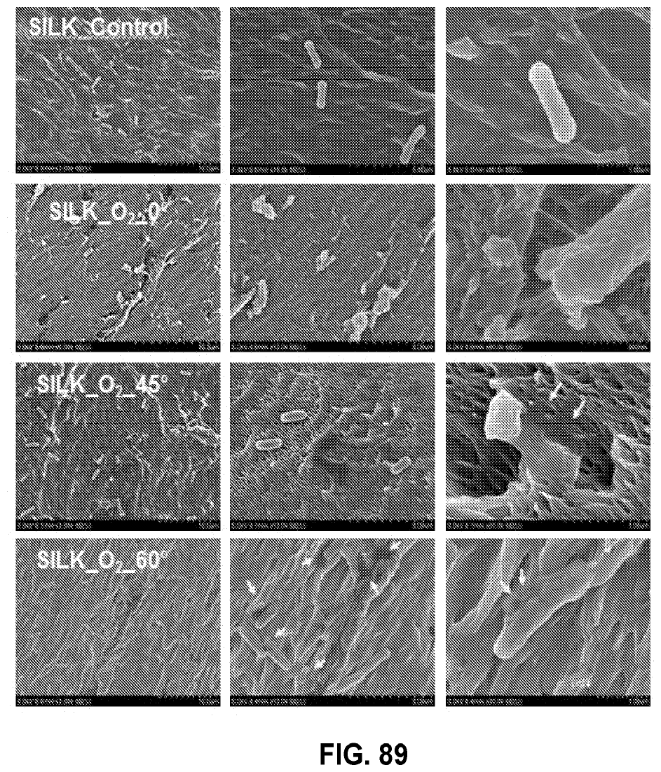

FIG. 47. Atomic Concentration of 0 on Si after Codeposition Organized by Flux Ratio.

FIG. 48. Atomic Concentration of Zn on Si after Codeposition Comparing Fluence.

FIG. 49. 49 Atomic Concentration of 0 on Si after Codeposition Comparing Fluence.

FIG. 50. 50 Survey XPS scans of PDMS showing both a Virgin Surface and after Codeposition with 1000 eV O₂⁺ to Identify the Relative Peaks, and show how surfaces change.

FIG. 51A-B. Region Scans of Zn (51A) and Si (51B) peaks after Codeposition with 1000 eV O₂⁺.

FIG. 52. Atomic Concentration of Zn on PDMS after Codeposition Organized by Flux Ratio.

FIG. 53. Atomic Concentration of Zn on PDMS after Codeposition Organized by Flux Ratio.

FIG. 54. Atomic Concentration of Zn on PDMS after Codeposition Comparing Fluence.

FIG. 55. Atomic Concentration of Zn on PDMS after Codeposition Comparing Fluence.

FIG. 56. Water Droplet Contact Angle Measurement on Si.

FIG. 57. Contact Angle Measurements on Si after Codeposition with Ar⁺.

FIG. 58. Contact Angle Measurements on Si after Codeposition with O₂⁺.

FIG. 59. Zn Concentration Effects on Contact Angle on Si.

FIG. 60. Water Droplet Contact Angle Measurement on Si.

FIG. 61. Contact Angle Measurements on PDMS after Codeposition with O₂⁺.

FIG. 62. Zn Concentration Effects on Contact Angle on PDMS.

FIG. 63A-B. Results of irradiation synthesis of nanostructured natural nanocellulose materials with enhanced multi-functional properties. FIG. 63A-B shows before/after SEM images (low and high resolution) of chitosan cellulose material synthesized with Au nanoparticles and Ag nanoparticles irradiated with DPNS at 1-keV Ar⁺ normal incidence.

FIG. 64. Results of irradiation synthesis of nanostructured natural nanocellulose materials with enhanced multi-functional properties. X-ray diffraction data showing enhanced peak from embedded metal nanoparticles in the irradiated matrix

FIG. 65. Results of irradiation synthesis of nanostructured natural nanocellulose materials with enhanced multi-functional properties. FIG. 65 shows contact angle data for chitosan samples with corresponding diagram to illustrate the transition of chitosan from hydrophobic to hydrophilic properties after irradiation at room temperature.

FIG. 66. Results of irradiation synthesis of nanostructured natural nanocellulose materials with enhanced multi-functional properties. FIG. 66 shows SEM before/after of bacterial nanocellulose integrated with Ag nanoparticles. Note the dramatic transformation from BNC cellulose to pillar super nanostructures.

FIG. 67. Results of irradiation synthesis of nanostructured natural nanocellulose materials with enhanced multi-functional properties. FIG. 67 shows contact angle data for BNC samples with corresponding diagram.

FIG. 68. Diagram of sequence of synthesis of natural cellulose irradiated by DPNS

FIG. 69. DPNS synthesis irradiation conditions

FIG. 70. Results of liquid plasma directed simultaneous fabrication of nanoparticles and nanopatterns for multifunctional natural biomaterials. The SEM images show chitosan and cellulose film with gold nanoparticles and nanopatterns before and after plasma jet treatments in solution.

FIG. 71. Cartoon of the sequence of directed simultaneous fabrication of nanoparticles and nanopatterns for multifunctional biomaterials via liquid plasma method.

FIG. 72. Shows photograph of plasma jet equipment.

FIG. 73 shows a schematic representation of the main goal of surface modification of these 3D silk tubular scaffolds. On the right, the figure corresponds to Dalby et al., 2012.

FIG. 74 shows a schematic representation of different irradiation approaches by DPNS combining neutral and reactive gas species.

FIG. 75 shows SEM images of a raw surface of a silk tube scaffold.

FIG. 76 shows SEM images of outer surface of the 3D silk scaffold.

FIG. 77 shows SEM images of the inner surface of the 3D silk scaffold.

FIG. 78 shows SEM images of the inner and outer surface of the 3D silk scaffold, side by side.

FIG. 79 shows SEM images of flat silk scaffolds irradiated by DPNS.

FIG. 80 shows AFM images and roughness (Ra and RMS) quantification of the silk scaffolds of FIG. 79.

FIG. 81 shows quantitative data obtained through analysis of SEM images by Image J.

FIG. 82 shows an example of topography analysis using Image J.

FIG. 83(A) shows images of pristine bacterial nanocellulose (BC).

FIG. 83(B) shows images indicating that treatment of bacterial cellulose (BC) with Argon at a fluence of 1E18 ions/cm² creates nanopillars-like structures at the surface of the material.

FIG. 84(A) shows the Young Modulus of 8.77 for argon-treated BC.

FIG. 84(B) shows that argon-treated BC is rich in C—C/C—H bonds.

FIG. 84(C) shows images demonstrating that argon-treated BC is superhydrophilic.

FIG. 85(A) Image showing E. coli on control bacterial nanocellulose

FIG. 85(B) Image showing argon-treated BC's bactericidal activity against E. coli showing that irradiated bacterial cellulose kills E. coli upon contact.

FIG. 86 shows AFM images showing that the resultant wrinkle structure in argon-treated PDMS depends on the ion energy, and it is irrespective of the initial material stiffness.

FIG. 87(A) shows AFM images of PDMS prepared at ratios of 10:1, 30:1, and 50:1 of the base-to-catalyst agent show that the wrinkle wavelength decreases with the angle of incidence increases.

FIG. 87(B) shows a graph depicting power spectral density for AFM images in 87(A), and showing that the spatial frequency of the wrinkles depends on the initial stiffness of the PDMS.

FIG. 87(C) shows the RMS height remains similar at an angle of incidence of 0 and 45 degrees for rigid PDMS, but increases for soft substrates at normal incidence.

FIG. 88(A) shows scanning electron microscope images showing a similar wrinkle structure on a film of PDMS prepared with a base to catalyst ratio of 10:1.

FIG. 88(B) graph indicates that the effective Young Modulus for the experimental samples in 88(A) shows a statistically significant difference between argon versus oxygen and krypton irradiation of PDMS(10:1) (*p-value<0.01).

FIG. 88(C) shows images of after contact angle for PDMS(10:1) treated with argon, oxygen, and krypton at two incidence angles.

FIG. 89 shows SEM images of oxygen-treated silk's bactericidal activity against E. coli, showing that irradiated samples can disturb the bacteria membrane to produce leaks of internal bacterial content.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Nanoscale domains,” as used herein, refers to features characterized by one or more structural, composition and/or phase properties having relatively small dimensions generated on the surface of a substrate. Nanoscale domains may refer to relief features and/or recessed features such as trenches, nanowalls, nanocones, nanoplates, nanocolumns, nanoripples, nanopillars, nanorods, nanowires, nanotubes, surface porous structures, and/or quantum dots. Nanoscale domains may refer to discrete crystalline domains, compositional domains, distributions of defects, and/or changes in bond hybridization. Nanoscale domains include self-assembled nanostructures. In embodiments, for example, nanoscale domains refer to surface depths or structures generated on a surface having dimensions of less than 1 μm, less than or 500 nm, less than 100 nanometers, or in some embodiments, less than 50 nm. In an embodiment, nanoscale domains refer to a domain in a thermally stable metastate.

“Surface geometry” refers to a plurality nanoscale domains positioned on the surface of a substrate. In embodiments, for example, nanofeatured surface geometry is a periodic or semi-periodic spatial distribution of nanoscale domains. For example, nanofeatured surface geometries include topology, topography, spatial distribution of compositions, spatial distribution of phases, spatial distribution of crystallographic orientations and/or spatial distribution of defects. Surface geometries of some aspects are useful for providing a selected multifunctional bioactivity, a selected physical property or a combination thereof.

“Selected function” refers to an enhancement of in vivo or in vitro activity with respect to a plurality of biological or physical processes. In embodiments, for example, selected function is enhancement of a biological property which includes an enhancement in cell adhesion activity, cell proliferation activity, cell in-migration activity, cell differentiation activity, anti-bacterial activity, bactericidal activity, anti-inflammatory activity, osseoconductive activity, osseointegration activity, immuno-modulating activity during acute or chronic inflammation, hemocompatibility, or any combination of these. “Selected function” also includes an enhancement of a physical property such as surface hydrophilicity, surface free energy, surface hydrophobicity, sensing, drug transport, surface acidity, surface basicity, or combinations thereof.

“Directed energetic particle beam,” as used herein, refers to a stream of accelerated particles. In embodiments, the directed energetic particle beam is generated from low-energy plasma. In some embodiments, directed energetic particle beam is a focused or broad ion beams capable of delivering a controlled number of ions to a precise point or area upon a substrate over a specified time. Directed energetic particle beam may include ions and additional non-ionic particles including subatomic particles or neutral atoms or molecules. In embodiments, directed energetic particle beams provide individual ions to the target location. Examples of directed energetic particle beams include focused ion beams, broad ion beams, thermal beams, plasma generated beams, optical beams and radiation beams.

“Beam property” or “beam parameter” refer to a user or computer controlled property of beam, for example, an ion beam. Beam parameter may refer to incident angle with a target substrate, fluence, energy, flux, beam composition and ion species. Beam parameters may be adjusted to provide selected interactions between the beam and the target substrate to generate specific nanostructures or enhance specific properties of the substrate including rate of bioresorption. Beam parameters may be controlled by a variety of means, including adjustments to electromagnetic devices in communication with the beam, adjusting the gas or energy source used to generate the beam or physical positioning of the beam in reference to the target.

“Vertical spatial dimension” refers to a measure of the physical dimensions of a nanoscale domain perpendicular or substantially perpendicular to the planar or contoured surface of a substrate. In embodiments, vertical spatial dimension refers to a height or depth of a nanoscale domain or the mean depth of a surface modification, for example, a crystalline or compositional domain.

“Lateral spatial dimension” refers to a measure of the physical dimensions of a nanoscale domain parallel or substantially parallel to the planar or contoured surface of a substrate.

“Polymer” refers to a molecule composed of repeating structural units connected by covalent chemical bonds often characterized by a substantial number of repeating units (e.g., equal to or greater than 3 repeating units, optionally, in some embodiments equal to or greater than 10 repeating units, in some embodiments greater or equal to 30 repeating units) and a high molecular weight (e.g. greater than or equal to 10,000 Da, in some embodiments greater than or equal to 50,000 Da or greater than or equal to 100,000 Da). Polymers are commonly the polymerization product of one or more monomer precursors. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits, and include random, block, brush, brush block, alternating, segmented, grafted, tapered and other architectures. Useful polymers include organic polymers or inorganic polymers that may be in amorphous, semiamorphous, crystalline or semi-crystalline states. Cross linked polymers having linked monomer chains are useful for some applications.

Polymers can include “block copolymers” which are a type of copolymer comprising blocks or spatially segregated domains, wherein different domains comprise different polymerized monomers, for example, including at least two chemically distinguishable blocks. Block copolymers may further comprise one or more other structural domains, such as hydrophobic groups, hydrophilic groups, etc. In a block copolymer, adjacent blocks are constitutionally different, i.e. adjacent blocks comprise constitutional units derived from different species of monomer or from the same species of monomer but with a different composition or sequence distribution of constitutional units. Different blocks (or domains) of a block copolymer may reside on different ends or the interior of a polymer (e.g. [A][B]), or may be provided in a selected sequence ([A][B][A][B]). Polymers can include “diblock copolymer” which refers to block copolymer having two different polymer blocks. “Triblock copolymer” refers to a block copolymer having three different polymer blocks, including compositions in which two non-adjacent blocks are the same or similar. “Pentablock” copolymer refers to a copolymer having five different polymer including compositions in which two or more non-adjacent blocks are the same or similar.

“Hydrophobic” refers to a property of a functional group, or more generally a component of a compound, such as one or more polymer side chain groups, which are immiscible with polar compounds, including, but not limited to, at least one of the following: water, ionic liquid, lithium salts, methanol, ethanol, and isopropanol. In a specific embodiment, for example, “hydrophobic” refers to a property of a functional group, or more generally a component of a compound, such as one or more polymer side chain groups, which are immiscible with at least one of the following water, methanol, ethanol, and isopropanol. In an embodiment, for example, polystyrene, poly(methyl methacrylate), poly(ethylene), poly(propylene), poly(butadiene), and poly(isoprene) are examples of hydrophobic polymer side chains.

“Hydrophilic” refers to a property of a functional group, or more generally a component, of a compound, such as one or more polymer side chain groups, which exhibit miscibility at certain relative concentrations with polar compounds including, but not limited to, at least one of the following: water, ionic liquid, lithium salts, methanol, ethanol, and isopropanol. In a specific embodiment, for example, “hydrophilic” refers to a property of a functional group, or more generally a component, of a compound, such as one or more polymer side chain groups, which exhibit miscibility with at least one of the following water, methanol, ethanol, and isopropanol.

Some polymers useful in the present compositions are derived from polymerization of a monomer selected from the group consisting of a substituted or unsubstituted norbornene, olefin, cyclic olefin, norbornene anhydride, cyclooctene, cyclopentadiene, styrene and acrylate. Some polymer backbone groups useful in the present compositions are obtained from a ring opening metathesis polymerization (ROMP) reaction. Polymer backbones may terminate in a range of backbone terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO_{2R}³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylate, or catechol; wherein each of R³⁰-R⁴² is independently hydrogen, C₁-C₁₀ alkyl or C₅-C₁₀ aryl.

Some polymers have polymer side chain groups which include unsubstituted or substituted polyisocyanate group, polymethacrylate group, polyacrylate group, polymethacrylamide group, polyacrylamide group, polyquinoxaline group, polyguanidine group, polysilane group, polyacetylene group, polyamino acid group, polypeptide group, polychloral group, polylactide group, polystyrene group, polyacrylate group, poly tert-butyl acrylate group, polymethyl methacrylate group, polysiloxane group, polydimethylsiloxane group, poly n-butyl acrylate group, polyethylene glycol group, polyethylene oxide group, polyethylene group, polypropylene group, polytetrafluoroethylene group, and polyvinyl chloride group. Some polymer side chain groups useful in the present compositions comprise repeating units obtained via anionic polymerization, cationic polymerization, free radical polymerization, group transfer polymerization, or ring-opening polymerization. A polymer side chain may terminate in a wide range of polymer side chain terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO_{2R}³⁶, —OR³⁷, —SR³⁸, —NR³⁹R⁴⁰, —NR⁴¹COR⁴², C₁-C₁₀ alkyl halide, phosphonate, phosphonic acid, silane, siloxane acrylate, or catechol; wherein each of R³⁰-R⁴² is independently hydrogen or C₁-C₅ alkyl.

“Polymer blend” refers to a mixture comprising at least one polymer, such as a block copolymer, e.g., brush block copolymer, and at least one additional component, and optionally more than one additional component. In some embodiments, for example, a polymer blend of the invention comprises a first brush block copolymer and one or more electrochemical additives. In some embodiments, for example, a polymer blend of the invention further comprises one or more additional brush block copolymers, homopolymers, copolymers, block copolymers, brush block copolymers, oligomers, electrochemical additives, solvents, metals, metal oxides, ceramics, liquids, small molecules (e.g., molecular weight less than 500 Da, optionally less than 100 Da), or any combination of these. Polymer blends useful for some applications comprise a first block copolymer, such as a brush block copolymer or a wedge-type block copolymer, and one or more additional components comprising block copolymers, brush block copolymers, wedge-type block copolymers, linear block copolymers, random copolymers, homopolymers, or any combinations of these. Polymer blends of the invention include mixture of two, three, four, five and more components.

“Synthetic polymer” can include a polyolefin; a silicone; a polyacrylate or polymethacrylate; a polyester; a polyether; a polyamide, and a polyurethane. The polyolefin may be selected from polypropylene, polyethylene, poly(tetrafluoroethylene) and poly(vinyl chloride. The silicone may include poly(dimethyl siloxane). The polyacrylate or polymethacrylate may include poly(methyl methacrylate), poly(hydroxyethyl methacrylate). The polyester may include poly(ethylene terephthalate), poly(glycolic acid), poly-lactic acid, polydioxanone; or wherein the synthetic polymer is a polyether selected from the group consisting of polyether ether ketone and polyether sulfone.

“Cellulose” and “bacterial nanocellulose” refer to a polysaccharide biopolymer mostly produced by plants and micro-organisms and some marine animals. Nanocellulose may refer to at least three different types of nanocellulose materials, which vary depending on the fabrication method and the source of the natural fibers. These three types of nanocellulose materials are called nanocrystalline cellulose (NCC) microfibrillated cellulose (MFC), and bacterial cellulose (BC). Additional details regarding these materials are described in U.S. Pat. Nos. 4,341,807, 4,374,702, 4,378,381, 4,452,721, 4,452,722, 4,464,287, 4,483,743, 4,487,634 and 4,500,546, the disclosures of each of which are incorporated by reference herein in their entirety. Nanocellulose materials have a repetitive unit of β-1,4 linked D glucose.

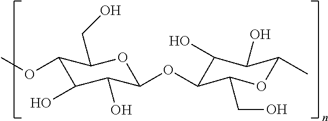

The integer values for the variable n relate to the length of the nanocellulose chains, which generally depends on the source of the cellulose and even the part of the plant containing the cellulose material. In some embodiments, n may be an integer of from about 100 to about 10,000, from about 1,000 to about 10,000, or from about 1,000 to about 5,000. In other embodiments, n may be an integer of from about 5 to about 100. In other embodiments, n may be an integer of from about 5000 to about 10,000. In embodiments, the nanocellulose chains may have an average diameter of from about 1 nm to about 1000 nm, such as from about 10 nm to about 500 nm or 50 nm to about 100 nm.

Bacterial nanocellulose produced by Acetobacter xylinum (Gluconacetobacter xylinus) has an intrinsic nanostructural hierarchy, purity, mechanical strength and chemical robustness. This bacterium produces extracellular cellulose under static culture condition in the form of highly reticulated net like structure along with the entrapped bacteria, media components and protein. The cellulose is purified using alkali treatment at boiling temperature. Bacterial nanocellulose is found to be about 20-100 nm in general. Characteristics of cellulose producing bacteria and agitated culture conditions are described in U.S. Pat. No. 4,863,565, the disclosure of which is incorporated by reference herein in its entirety. Bacterial nanocellulose particles are microfibrils secreted by various bacteria that have been separated from the bacterial bodies and growth medium. The resulting microfibrils are microns in length, have a large aspect ratio (greater than 50) with a morphology depending on the specific bacteria and culturing conditions.

“Chitosan” is a linear polysaccharide composed of randomly distributed β-(1→4)-linked D-glucosamine (deacetylated unit) and N-acetyl-D-glucosamine (acetylated unit). It is made by treating the chitin shells of shrimp and other crustaceans with an alkaline substance, like sodium hydroxide. Chitosan is produced commercially by deacetylation of chitin, which is the structural element in the exoskeleton of crustaceans (such as crabs and shrimp) and cell walls of fungi. The degree of deacetylation (% DD) can be determined by NMR spectroscopy, and the % DD in commercial chitosans ranges from 60 to 100%. On average, the molecular weight of commercially produced chitosan is between 3800 and 20,000 Daltons. A common method for the synthesis of chitosan is the deacetylation of chitin using sodium hydroxide in excess as a reagent and water as a solvent. The reaction occurs in two stages under first-order kinetic control. The amino group in chitosan has a pKa value of ˜6.5, which leads to a protonation in acidic to neutral solution with a charge density dependent on pH and the % DA-value. This makes chitosan water-soluble and a bioadhesive which readily binds to negatively charged surfaces such as mucosal membranes. Chitosan enhances the transport of polar drugs across epithelial surfaces, and is biocompatible and biodegradable.

“Polymer” refers may also refer to a protein polymer, such as a fibrous protein polymer molecule. “Fibrous protein substrate” or “fibrous protein” includes naturally occurring polymers e.g., biogenic polymers, e.g., proteins, which may be formed by processes known in the art, such as electrospinning. Exemplary biogenic polymers, e.g., polymers made in a living organism, e.g., fibrous proteins, for use in the devices and methods of exemplary embodiments include, but are not limited to, silk (e.g., fibroin, sericin, etc.), keratins (e.g., alpha-keratin which is the main protein component of hair, horns and nails, beta-keratin which is the main protein component of scales and claws, etc.), elastins (e.g., tropoelastin, etc.), fibrillin (e.g., fibrillin-1 which is the main component of microfibrils, fibrillin-2 which is a component in elastogenesis, fibrillin-3 which is found in the brain, fibrillin-4 which is a component in elastogenesis, etc.), fibrinogen/fibrins/thrombin (e.g., fibrinogen which is converted to fibrin by thrombin during wound healing), fibronectin, laminin, collagens (e.g., collagen I which is found in skin, tendons and bones, collagen II which is found in cartilage, collagen III which is found in connective tissue, collagen IV which is found in extracellular matrix protein, collagen V which is found in hair, etc.), vimentin, neurofilaments (e.g., light chain neurofilaments NF-L, medium chain neurofilaments NF-M, heavy chain neurofilaments NF-H, etc.), amyloids (e.g., alpha-amyloid, beta-amyloid, etc.), actin, myosins (e.g., myosin I-XVII, etc.), titin which is the largest known protein (also known as connectin), etc.

Silk fibroin is a fibrous protein secreted by the silkworm Bombyx mori, as well as by a number of different species of spiders. Used originally as a suture to facilitate wound approximation and/or ligation, silk has since been used for a range of clinical repair applications. Structurally, insect silk obtained from the cocoons of the silkworm B. mori is composed of two distinct proteins: the readily water-soluble sericin and fibroin, which is dissolvable in aqueous inorganic salt solutions. The solubilized fibroin could then be neutralized and dialyzed, resulting in a water-soluble form of fibroin. Solubilized fibroin thus obtained consists of segments primarily in the α-form, which are unstable and readily transition to the β-form, rendering the protein insoluble and giving it its fibrous structure and mechanical strength. Silk fibroin matrices, gels, and films have been previously shown to support cellular adherence and growth of a variety of different cell types either in their native state or as ECM-coated substrates in vitro. Silk fibroin films loaded with nerve growth factor (NGF) were previously shown to support adherence and neurite outgrowth from PC12 cells when used in nerve conduits.

Any type of silk fibroin may be used to make the silk fibroin substrate. Silk fibroin produced by silkworms is the most common and is generally preferred. However, there are many different silks, including spider silk, transgenic silks, genetically engineered silks, and variants thereof, that may alternatively be used. As used herein, the term “fibroin” includes silkworm fibroin and insect or spider silk protein (Lucas et al., Adv. Protein Chem 13: 107-242 (1958)). Preferably, fibroin is obtained from a solution containing a dissolved silkworm silk or spider silk. The silkworm silk protein may be obtained, for example, from Bombyx mori, and the spider silk is obtained from Nephila clavzes. In the alternative, the silk proteins can be obtained from a solution containing a genetically engineered silk, such as from bacteria, yeast, mammalian cells, transgenic animals or transgenic plants. See, for example, PCT Publication WO 97/08315 and U.S. Pat. No. 5,245,012, both of which are herein incorporated by reference in their entirety. The genetically engineered silk can, for example, comprise a therapeutic agent, e.g., a fusion protein with a cytokine, an enzyme, or any number of hormones or peptide-based drugs, antimicrobials and related substrates.

An aqueous silk fibroin solution may be prepared from the silk fibroin using techniques known in the art. Suitable processes for preparing silk fibroin solution are disclosed in PCT Application No. PCT/US2004/011199, and U.S. Provisional Application No. 60/856,297, filed Nov. 3, 2006, entitled “Biopolymer Photonic Crystals and Method of Manufacturing the Same,” both of which are herein incorporated by reference in their entirety. For instance, the silk fibroin solution may be obtained by extracting sericin from the cocoons of a silkworm silk, such as Bombyx mori. For example, B. mori cocoons can be boiled for about 30 minutes in an aqueous solution, preferably an aqueous solution having about 0.02M Na₂CO₃. The cocoons can then be rinsed with water, for example, to extract the sericin proteins. The extracted silk can then be dissolved in an aqueous solution, preferably an aqueous salt solution. Salts useful for this purpose include lithium bromide, lithium thiocyanate, calcium nitrate or other chemicals capable of solubilizing silk. Preferably, the extracted silk is dissolved in about 9-12 M LiBr solution. The salt may later be removed using, for example, dialysis.

The silk substrate may be used with any biological cells of or tissue. For instance, if the desired tissue is a cornea, then the tissue-specific cells should be biological cells having corneal properties or being capable of being used with other biological cells having corneal properties. Suitable tissue-specific cells include, but are not limited to, stem cells, fibroblasts, endothelial, epithelial, adipose cells capable of generating tissue-specific extracellular matrix, and combinations thereof.

“Substrate” refers to the target of an ion beam as described herein. In embodiments, substrates may comprise any material capable of forming nanostructures. Examples of substrates include polymers, including synthetic polymers, and polysaccharide biopolymers.

“Porosity” or “surface porous structure” refers to substrates or surfaces having individual or networked voids at or near the surface of the substrate. Porosity may be nanoscale, microscale or larger. As described herein, substrates may have porosity prior to any plasma treatment (e.g. porosity formed during substrate formation such as sintering). In some embodiments, pores may be formed, enlarged or altered by the treatment of directed plasma, including forming nanopatterns on interior pore surfaces or walls between individual pores.

“Metal or metal oxide nanoparticles” include gold nanoparticles, silver nanoparticles, zinc nanoparticles, zinc sulfide nanoparticles, zinc oxide nanoparticles, copper nanoparticles, platinum nanoparticles, cobalt nanoparticles, cobalt ferrite nanoparticles, ferric oxide nanoparticles, yttrium nanoparticles, zirconium nanoparticles, ruthenium nanoparticles, palladium nanoparticles, rhodium nanoparticles, iridium nanoparticles, or any combinations or oxides thereof. Silver or other metal nanoparticles may be formed in situ on a surface. For instance, a method comprises providing a suspension comprising finely dispersed particles of a metal compound such as a metal salt in which a surface is immersed or contacts the suspension, followed by addition of a reducing agent by methods described herein for a specified period of time or until the metal salt is reduced and forms nanoparticles, that are predominantly mono-disperse, and the nanoparticles attach or adhere to the surface. “Nanoparticles” as described herein are generally uniform in size, generally spherical, and can be preformed or made in situ. Although most particles are spherical other types of shapes can also form and be present in the compositions of the present invention.

“Multiplexing” refers to simultaneously modifying the target substrate in more than one way, for example, by providing two or more directed particle beams at the substrate having different properties, for example, to generate or modify at least one nanoscale domain (e.g. nanoscale features, crystalline domains, compositional domains, distributions of defects, changes in bond hybridization. In some embodiments, for example, a single directed particle beam may have one or more beam properties to generate or modify multiple nanoscale domains on the substrate. In embodiments, multiple direction particle beams are generated from the same plasma source.

The technology as described in the present disclosure includes an advanced nanomanufacturing process as described herein, advanced tools particular for this process and a number of unique nano-scale structures generated as a result of the processing.

In one embodiment, provided is an atomic-scale additive nanomanufacturing process capable of transforming materials with multi-functional properties without the need for expensive heat cycles, toxic chemical processes or thermodynamic limitations of material compatibility in processing. The interface between plasma and material becomes an open thermodynamic system driven far from equilibrium by a rich variety of physical mechanisms, including high-energy kinetic disordering, compositional phase dynamics, and the emergence of metastable material states. The instabilities that arise due to these mechanisms lead to the evolution of well-ordered nanostructures, the compositional and morphological characteristics of which dictate the material properties.

“Directed energetic particle beams” are drawn from a low-temperature plasma (gas discharge) in a manner that controls the energy, species and intensity of the respective beams from the aforementioned plasma. This technique may be called directed plasma nanosynthesis (DPNS), directed irradiation synthesis (DIS), Direct Seeded Directed Plasma Nanosynthesis (DSDPNS), or DSPNS (directed soft plasma nanosynthesis) herein. The particles may be combined with additional reactive atoms and/or surfactants that interact with material surface inducing variation in a number of properties including: surface chemistry, composition, topography, topology, charge density and bond hybridization. In some cases the technology can manipulate these properties independently providing for multi-functionality on the material surface without modification to the bulk material. Depending on material type the energetic particles are selected both in mass and species to result in the desired material property (e.g. hydrophobicity, anti-bacterial for biomaterials, etc. . . . ). The material can be a polymer, metal, ceramic, or semiconductor and the synthesis can be done over large areas, at room temperature and over a short period of time (e.g. seconds). DPNS, DIS, DSDPNS, and DSPNS is designed to independently modify surface topography, composition and charge density yielding increase of surface energy and surface-to-volume ratios by factors of 50-100% and 100-1000, respectively. DPNS, DIS, DSDPNS, and DSPNS include a use of a plasma source enabling the modification of existing product materials (e.g. on a biomedical stent, implant device, etc.) improving their properties or synthesizing completely new class of materials. DPNS, DIS, DSDPNS, or DSPNS enables a single source that addresses the problematic use of thin-film coatings for bioactive interfaces, which can potentially lead to osteolysis and chronic inflammation. Coating disintegration and delamination is also a prevalent problem that cannot be solved with current synthesis approaches that include: electrophoretic deposition, anodization, electrolysis, reactive DC magnetron sputtering, RF plasma sputtering, and x-ray sintering among others. One of the primary issues with these conventional technologies is the formation of the interface between the coating and biomedical material substrate. Therefore, features of DPNS, DIS, DSDPNS, and DSPNS are: 1) low cost (e.g. they are a low-temperature process; heat cycles during synthesis make-up 30-40% of the current processing cost of surface modification techniques), 2) green and sustainable (does not require harsh chemicals for synthesis and can enhance usability of natural materials), and 3) scalable (particle irradiation can be conducted throughput levels of about 1012 micron 2/hr or a modification of a 6-inch wafer in about 10 seconds). Another added benefit and potentially disruptive approach is the ability to modify a surface composition and chemistry independent of the topography with high-fidelity. In other words, inducing a surface that can potentially enhance cell adherence and proliferation while repelling bacteria, for example.

“Directed energetic particle beams” include DPNS, DIS, DSDPNS, or DSPNS to produce nanostructures on the substrate surface. In first step, a substrate is provided in a fixture, not shown, where the directed energetic particle beam from a low temperature plasma may operate on the substrate with a surface. The directed energetic particle beam(s) from a low temperature plasma source are directed to the substrate surface in accordance with parameters and/or properties that correspond to a desired nanostructure topology. The parameter control may occur in an automated fashion, such as under the control of a numerical control device or special purpose computer, including a processing device and a memory containing programming instructions (not shown). In an optional step, additional beam(s) may be generated and directed to the surface of the substrate also in accordance with parameters and/or properties that correspond to a desired nanostructure topology. Optional step includes depositing one or more agents on the surface of the substrate.

“Directed energetic particle beams” can be derived from plasma processing sources known in the art, for example, Tectra GmbH Physikalische Instrumente (GENII PLASMA ION SOURCE) and Oxford Instruments (ISE 5 ion sputtering source). Also SVT Associates, Inc. provides the RF-6.02 Plasma Source. While the principles and methods for creating plasma sources are known, these plasma processing methods create only mono-directional particle beams, which limits their usage to flat, 2D surfaces. Methods for performing DPNS, DIS, DSDPNS, DSPNS (as defined herein) as 3D are described in, for example, U.S. Patent Application Ser. No. 62/483,105, “Directed Plasma Nanosynthesis (DPNS) Methods, Uses and Systems,” filed Apr. 7, 2017, the disclosure of which is incorporated by reference herein in its entirety.

“Directed energetic particle beams” include low temperature plasmas useful for the present invention such as gasiform plasmas with electron temperature under 10 eV, electron density typically from 1014 to 1024 m⁻³. In general, low temperature plasmas have a low degree of ionization at low densities. This means the number of ions and electrons is much lower than the number of neutral particles (molecules). Different particles inside the plasma, i.e. neutrals, ions and electrons, can have different temperatures or energies. Indeed, in many applications, the background gas is near room temperature. In this regard, gas phase reaction activation energy can be driven by electron impact rather than thermally and the substrate is not subjected to extreme heating, which is useful for functionalizing temperature sensitive substrates such as polymers. For “directed energetic particle beams” one or more beam properties is the gas, intensity, fluence, energy, flux, incident angle, species mass, charge, cluster size, molecule or any combinations thereof. In an embodiment, for example, the directed energetic particle beam comprises one or more ions, neutrals or combinations thereof. In embodiments, the one or more beam properties are the ion composition, neutral composition, the ratio of ion abundance to neutral abundance or any combination of these. In embodiments, the directed energetic particle beam is incident upon the substrate from a plurality of directions.

Using “directed energetic particle beams” nanostructures may be obtained as function of energetic particle species, fluence and incident angle with respect to the surface normal. For example, energetic particle species may include those obtained from gases such as Kr, Ar, Ne, Xe, H, He, O₂ and/or N₂. Fluence can be, for example, between 1×10¹⁷ to 1×10¹⁸ particles per second per square meter, but may vary from 0.1×10¹⁷ to 50×10¹⁷. In some embodiments, fluence is 1×10¹⁷, 2.5×10¹⁷, 5×10¹⁷, or 1×10¹⁸. Finally, incident angle may be varied in single degrees between the angles of 0 and 80 degrees, in some embodiments, for example, 30 degrees, 45 degrees, 60 degrees, and/or 80 degrees. In some embodiments, for example, the plasma-based source of the invention provides one or more directed particle beams having a distribution of incident angles, such as a distribution of incident angles characterized between 0 and 90 degrees with respect to the sample surface normal.

“Directed energetic particle beams” include methods of directing one or more additional beams onto the substrate surface, wherein the addition beams are one or more particle beams, radiation beams or a combination thereof. For example, the one or more additional beams are characterized by at least one beam property that differs from the one or more beam properties of the directed energetic particle beam. In an embodiment, the one or more additional beams are directed energetic particle beams. In embodiments, the one or more additional beams is a focused ion beam, a broad ion beam, a thermal beam, a plasma generated beam, an optical beam or any combination of these.

The invention may be further understood by reference to the following non-limiting Examples that expand on certain aspects and embodiments of the invention.

### Example 1: Fabrication of Metal-Oxide Thin-Films and Features on Dissimilar Materials Via Ion-Assisted Codeposition

The merging of metal oxides and polymers has a number of interesting potential applications that rely on the wettability, optical, and electronic properties of the surface. One challenge in the fabrication of these dissimilar materials is that the heat often used to create oxide nanostructures results in the thermal decomposition of the polymer. This requires creative approaches to successfully merge these materials. Many current approaches involve the separate creation of metal oxide nanostructures, followed by some process of embedding them in an uncured polymer. Previous work has shown that ion beams have been used to sputter deposit metals, pattern polycrystalline metals, controllably oxidize metal surfaces, and induce chemical changes in the surfaces of polymers. Presented here is a single step technique that draws on these, utilizing dual ion beams to deposit, oxidize, and pattern Zn on Si and PDMS.

Two ion beams are installed in a perpendicular configuration, with one normal to the substrate surface and the other parallel. The parallel beam passes over the substrate and impinges on a Zn target, sputter depositing the material onto the substrate. Simultaneously, the normal incidence beam impinges on the substrate surface, imparting energy and sputtering both the substrate material and the deposited Zn. The effects of changing the ion beam flux ratio (0.1-2.0), energy (500 eV and 1000 eV), species (Ar⁺ and O₂⁺ for substrate irradiation, Ar⁺ for sputter deposition), and fluence (1E17 ions/cm2 and 5E17 ions/cm2) are examined. These factors allow for the comparison of different deposition rates, chemical effects, and surface evolution stages in the synthesis of these functionalized surfaces. Surfaces are characterized by several ex-situ techniques: topography (AFM), chemistry (XPS), and wettability (static contact angle).

This technique has yielded a number of interesting surfaces. On Si, the formation of nanodots is seen under many processing parameters. These dots have no ordering, but their size (˜20-100 nm diameter) and spatial density (1-100's um-2) can be controlled by the flux ratio and ion energy. The codeposition on Si at higher total fluence is also shown to induce ripples in the Si surface in addition to the formation of nanodots, as is expected from normal incidence irradiation with the presence of small amounts of surface impurities. XPS has shown that the flux ratio can finely tune the amount of Zn deposited on the surfaces. On PDMS, all cases of irradiation, both with and without codeposition, have results in larger scale wrinkles to form on the surface (wavelength ˜500-1000 nm) that are similar to previous work with oxygen plasma immersion. Notably, these are created with both O₂⁺ and Ar⁺ ion beams. Atop this structure, the formation of nanodots is also seen. Again, these are not shown to have spatial ordering, but are larger than those seen on Si, ˜75-200 nm diameter. These form at fewer combinations of processing parameters and are seen to preferentially grown in the valleys of the wrinkle pattern, specifically as they get larger. The ability to control the size and density of nanodots on PDMS with processing parameters is less clear than on Si.

This work represents a relatively fast, scalable, low-temperature, single-step process to grow and functionalize metal-oxide nanostructures on polymers. The ability to functionalize flexible, transparent substrates with metal-oxide nanostructures offers exciting applications in areas such as flexible and wearable electronics, gas sensors, biosensors, and photonics. 1.1 ZnO Properties and Applications

Zinc oxide is a versatile semiconductor that has been studied for many decades. It is a promising candidate for optoelectronics applications due to its wide direct band gap of 3.4 eV. ZnO is considered to be a superior UV emitter than the more commonly used GaN due to its 60 meV exciton binding energy [1,2]. Nanorods have also been shown to be effective light emitting diodes over the visible spectrum [3]. Thin films have been used as surface acoustic wave (SAW) filters and piezoelectrics for ultrasonic transducer arrays and other microelectromechanical systems (MEMS) [4,5]. In the area of transparent electronics, ZnO, in the form of InGaO₂(ZnO)5 has been shown to be a very effective transparent field-effect transistor (TFET) with an on-to-off current ratio of ˜106 [6]. ZnO also shows potential in spintronics, with ferromagnetism induced via transition metal doping [7,8]. In addition to these lucrative applications, ZnO has the advantage of being abundantly available and relatively affordable. A number of important properties of ZnO are summarized below in Table 1 [9].

TABLE 1
Wurtzite ZnO Properties [9]

| Property | Value |
|---|---|
| Stable phase at 300K | Wurtzite |
| Lattice spacing | |
| a₀ | 0.32495 nm |
| c₀ | 0.52069 nm |
| Density | 5.606 g/cm³ |
| Melting point | 1975° C. |
| Static dielectric constant | 8.656 |
| Energy gap | 3.4 eV, direct |
| Exciton binding energy | 60 meV |

One area of promising applications currently being developed is for chemical sensors in gaseous and biological systems. Highly sensitive devices for the detection of H2S have been created with Sn₂O—ZnO films [10] and for humidity, CO, and H₂ using CuO/ZnO thin films [11]. While many functional metal-oxides are viable for such applications, ZnO is highly desirable due to its biocompatibility [12]. ZnO nanoparticles and other features have been used for a wide variety of sensing applications that utilize either their ability to scatter light or their change in conductivity due to particle interactions. Applications include nanoparticles for immunosensors, films for H₂O₂ [13], “nanocombs” for blood glucose [14], and films for Ochratoxin-A (OTA), a mycotoxin found in food products [15]. An astounding feature of these detectors is the physical scale on which they operate. The OTA sensor mentioned above utilized ZnO nanoparticles of just 5 nm diameter on ITO glass.

The aforementioned applications utilize ZnO in a number of forms, including single and polycrystalline bulk material, thin films, rods, wires, ribbons, and dots [3,5,11-14,16]. These are fabricated by a variety of techniques, many of which require high temperatures or corrosive chemical treatments [9,16-22]. ZnO is commonly formed through the oxidation of Zn at temperatures of 200-1000° C. depending on the application [1,6,12]. Another technique of oxidation and deposition is from solutions with a significant range in pH values depending on the desired surface features [23]. Other techniques include chemical vapor deposition (CVD), metal organic vapor phase epitaxy, electrodeposition, and vapor-liquid-solid growth [1,3].

Flexible Substrates and the Future of Medical Devices

The field of medical research is one of the largest and fastest growing fields in the world. A significant focus within medical research is developing new monitoring and diagnostic tools and techniques, requiring materials that are compatible not only with biochemistry, but also the movement of the body. This naturally leads to the processing of flexible materials to perform in applications, such as bio-sensing, while meeting both of these needs. A number of materials are currently being researched for these uses, as well as for other applications like flexible electronics systems [24,25], including indium tin oxide (ITO) [23,26,27], poly(methyl methacrylate) (PMMA) [28,29], chitosan [30,31], and polydimethylsiloxane (PDMS) [32-34]. Of these, PDMS has received considerable attention due to a number of favorable properties. Fabrication consists of mixing a liquid base and liquid polymer, followed by low-temperature curing, which allows it to easily be molded into almost any geometry. Additionally, it is transparent, biologically inert, stable under moderate conditions, and durable, yet flexible [34].

PDMS is a polymer chain, with base unit (C₂H₆OSi)ₙ composed of a Si and O backbone and CHs groups attached to each Si atom. It features a shear elastic modulus of ˜250 kPa and a low glass transition temperature of ˜−125° C. [35]. It also has a safe temperature range of −45-200° C., above which the polymer will begin to dissociate, limiting certain applications [36]. The compound is stable in the presence of materials with moderate pH value, but can be broken down by strong acids and bases.

A key approach to modifying the properties of flexible substrates for biomedical uses is to directly or, through a series of steps, create thin films or nanostructures on their surface. This stems from the bulk of the material already possessing the desired mechanical properties, but possibly lacking other desired properties that dictate how the body interacts with it. These interactions are largely dictated by the surface properties of the material, as it is in direct contact with bodily tissue and fluid. Thin films are generally much more flexible than their bulk counterparts, but suffer from delamination from flexible substrates due to factors such as drastically different Young's moduli and thermal expansion coefficients [37]. This makes individual features, such as dots, needles, and pillars attractive options to pursue [38,39]. Surface modification has led to PDMS being used in a number of medical devices, including contact lenses, cochlear implant coatings, artificial skin, blood pumps, catheters, and denture liners [40]. Other applications for PDMS currently being researched include magnetic actuators [41] and catalysts [39].

The surface modification of PDMS is currently achieved through a number of techniques designed to be compatible with the material's temperature and chemical constraints. A common technique takes advantage of the curing process to embed features into the surface [32]. In one example, metalorganic chemical vapor deposition (MOCVD) was used to grow a 200 nm seed layer on SiO₂. This was then subject to a hydrothermal patterning technique involving Zn(NO3)2□6H2O and C6H12N4 to synthesize nanowires, with 1,3-diaminopropane added to taper them. Uncured PDMS was cast over the nanowire containing substrate, and allowed to seep into the nanowires. The PDMS was then cured and peeled off, taking the nanowires with it. Though this procedure is successful, it requires numerous steps and is time consuming. Another fabrication method is nanoimprint lithography, which again requires a number of steps to fabricate and transfer materials to embed structures in polymers [27]. This processes is additionally limited by the minimum feature size of the initial lithography technique.

1.3 Material Processing and the Role of Ion Beam Technology

The ability to modify surface properties is central to both of the previous subjects and one of the most important factors in biomedical device fabrication and performance. Two of the most crucial factors in determining surface properties are chemistry and morphology. Material surface modification has been shown to offer control over many properties, including wettability, chemical, and optical properties for applications in areas such as solar energy, gas sensors, and biosensors [27,42-44]. A significant number of diverse material synthesis methods have been developed to this end, focusing on nanoscale modification, with a variety of strengths and weaknesses. Several of these were highlighted above in the context of ZnO and PDMS, and others include physical vapor deposition [45], dual-plasma-enhanced metalorganic vapor deposition [46], chemical solution deposition [33], and post fabrication introduction of features into uncured polymers [34,47].

Another technique that has a long history in the semiconductor business, but is still growing in usage in many other industries, is plasma processing. At the most basic level this involves the bombardment of a surface with energetic ions, electrons, neutral gas atoms, and free radicals to induce changes in surface properties [48-50]. Plasma immersion eventually led to the development of broad-beam (and later, focused beam) ion sources. Though many designs exist, these tools create parallel beam of particles impinging on a surface, expanding exposure control to parameters like incident beam angle and precise acceleration energy [51,52].

When an ion collides with a surface, a number of events occur. Low energy ions may bounce off the surface or become physisorbed, imparting some amount of energy either way [53]. For incident ions with high enough energy to enter the material, a collision cascade is initiated, during which the energy from the initial ion is dispersed within a small volume [53]. This can cause a great number of Frenkel pairs to form if the target material is crystalline [54]. It is also theorized that the thermal spike within this volume can cause localized melting occurs, which may relieve a small amount of internal stress, similar to annealing, and allow for phase separation [53]. In multi-component systems, such as polymers, this will also break interatomic bonds, creating a much more chemically active surface [42]. Chemical activity can also be significantly affected by using chemically reactive gas species, such as O₂, as opposed to inert noble gases, such as Ar. Within the collision cascade, some of the momentum will be redirected back toward the surface, causing some of the material to be ejected from the surface in a process known as sputtering [55,56]. This, along with increased surface mobility, induces a significant mechanism in describing surface evolution during ion irradiation: mass redistribution [57-59].

Through decades of development, these mechanisms have been developed into a large number of material modification and analysis techniques. Sputtering has been developed into both material deposition tools, such as magnetron sputtering guns, and surface cleaning techniques, like ashing to remove photoresist in lithography [28,60]. Focused ion beams are used to etch very thin samples for transmission electron microscopy. Analysis of moment transfer between ion beams and surfaces have yielded highly surface sensitive chemical analysis techniques such as forward- and backward-ion scattering spectroscopy. Ions and neutral atoms sputtered from the surface can also be analyzed to produce surface composition in secondary ion mass spectroscopy (SIMS) and secondary neutral mass spectroscopy (SNMS), respectively [61,62].

Due to the early adoption and significant usage of this technology by the semiconductor industry, it follows that the most significant work has been performed on crystalline silicon. One of the earliest known discoveries of ion beam surface patterning occurred on glass in 1962, where ionized air produced ripple patterns with angle-dependent periodicity [63]. Since then, a number of studies have shown the angle of incidence can cause ripples to form in two distinct modes: parallel mode at high incident, grazing angles, where the apparent propagation direction of the ripples is parallel to the direction of the ion beam, and perpendicular mode at low angles near normal incidence, in which the propagation direction of the ripples appears to be perpendicular to the direction of the ion beam [64,65]. An additional manner of patterning Si concerns normal incidence irradiation. Early work was inconclusive on the results, with some work showing no significant changes and others showing dot and hole formation [66-69]. More recent work has shown that surface patterns from normal incidence irradiation of Si occurs in the presence of impurities, with a threshold below which no patterns form [70-74].

A number of theoretical models have been proposed to explain the observed self-organized behavior. One of the earliest and still most referenced of these is the Bradley-Harper model [75], a continuum model that predicts ripple formation at low fluences, and is based on Sigmund's theory of sputtering [56]. This is based on the idea that an initially rough surface has relative high points (peaks) and low points (trough). When an ion impacts near a peak, some of the its energy may distribute to the peak, but most will be distributed away. Likewise, an ion impacting near a trough will distribute its energy toward the trough. In this manner, more energy reaches troughs than peaks and thus material is sputtered away from them at a higher rate. This effect is responsible for surface instability (on Si) and drives surface evolution. The original Bradley-Harper model has been updated many times over the years, with new terms, such as the Kuramoto-Sivashinsky (KS) equation, accounting for non-linear effects like mass redistribution at higher fluences [76,77].

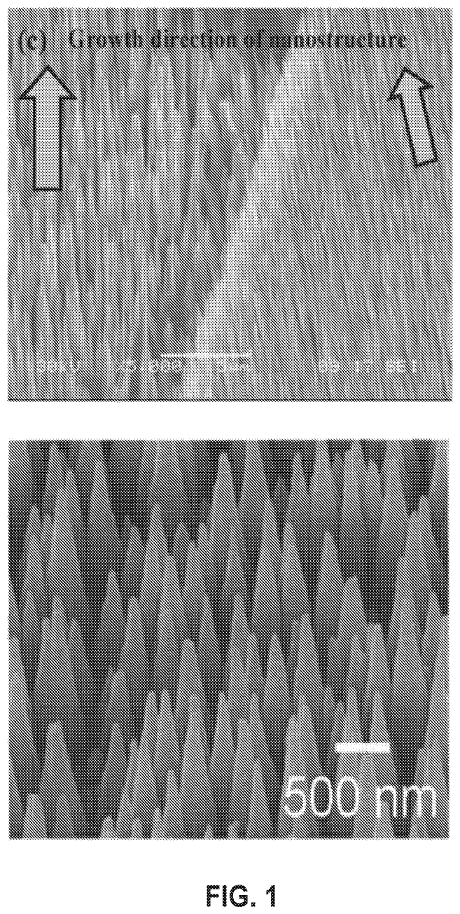

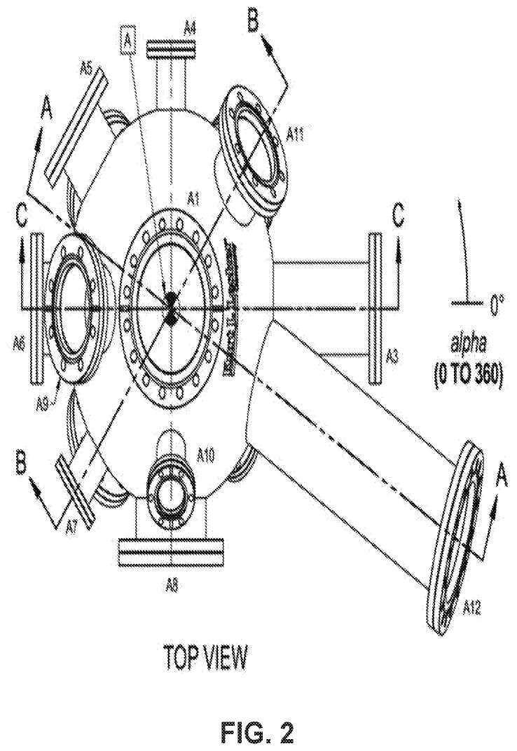

Plasma and ion beam interactions have also been studied on Zn, ZnO, and PDMS. Several groups have shown that ion beam irradiation of Zn produces nanostructure growth. Normal incidence irradiation of polycrystalline Zn with 600 eV Ar⁺ ions has been shown to result in needle growth that is dependent on grain crystallography, which is shown in FIG. 1 (left) [78].

Additionally, 6 keV O₂⁺ irradiation of Zn has been shown to form cones [79] (FIG. 1, right). It is believed that ZnO formed in random areas on the surface, and, with a lower sputter yield than pure Zn, was removed at a lower rate, allowing the surrounding material to be removed and forming cones. Neither of these experiments directly measured the flux or fluence of the ion beam, instead reporting equipment settings.

Multiple studies have been performed exploring the usage of oxygen ions as a means to controllably oxidize metal surfaces at low temperatures. Metal oxide films were produced on Mo, W, Nb, and Ta with 1 keV O₂⁺ ions [80,81]. Chemical analysis with XPS showed that the oxidation state was fluence dependent and eventually reached an equilibrium stoichiometry in each system as the oxidation and sputtering rates balanced. Additional studies have shown similar results with lower energy ions in the range of 20-600 eV [82,83].

Oxygen plasma immersion has been shown to drastically affect the wettability of PDMS and other polymers [28,40,84-86]. In one study, 200 nm of PDMS was cured on SiO₂ substrates and exposed to both radio frequency and microwave oxygen plasmas at 40 W. These were then aged in various environments and analyzed periodically. XPS showed that oxygen irradiation creates an oxidized layer of 130-160 nm, depending on exposure time. The incident oxygen broke PDMS molecules and created a high concentration of SiO_{3,4} groups in this layer, which is responsible for the polymer shifting from hydrophobic to hydrophilic. This was characterized through water droplet contact angle measurements, with an observed shift from ˜96° to less than 30° [42]. This effect was not permanent, however, and the mechanism for hydrophobic recovery was also explored. One conjecture that the work disproved was that contamination from the environment penetrated the surface and aided in recovery. Samples of PDMS were aged in atmosphere, argon, and vacuum environments. These conditions showed no observable differences, and the effect is attributed to migration of molecules within the sample over time, dispersing the concentration of SiO_{3,4} near the surface. Though this work presented this as a means of artificially aging PDMS, this could be an effective means of creating flexible materials that require time-dependent characteristics, such as the interaction of biomaterials during a healing process.

Other work with PDMS also exposed the surface to oxygen plasma. This work adjusted the power used to generate the plasma and the length of time the PDMS was immersed in it. This resulted in the formation of waves across the surface that was attributed to the formation of a silica layer, via oxidation from the plasma, with different mechanical properties [87]. These were characterized by wavelengths of 100's nm. These structures were shown to drastically decrease the contact angle of water in wettability studies from ˜110° to <20° [88].

Overall, ion beam irradiation has demonstrated the capability to produce a wide variety of features such as rods, ripples, and dots [78,89-94] and produce significant chemical changes [80,95-97]. These are highly tunable through the manipulation of processing conditions, including incident angle, gas species, energy, flux, fluence, and temperature. Overall, the expansion of plasma and ion beam processing of materials is a potentially disruptive technology for industrial applications as it can provide a wide variety of surface property modifications, is generally low temperature, and is scalable, with the ability to simultaneously modify large surface areas at a quick rate.

On the Use of Dual Ion Beams

The breadth of applications of ion beams has inspired a few research efforts exploring the combination of these abilities. One such effort has focused on irradiating a single surface with two ion beams whose projections on the sample surface are perpendicular. This work explored the possibility of obtaining a superposition of ripple patterns on Au(001) surfaces with 500 eV Ar⁺ ions [98,99]. The results shows that dots were indeed formed on the surface, but had very poor ordering.

A separate experiment explored use two ion beams for very different purposes. One ion beam irradiated a Si sample at normal incidence, while a second passed parallel to the surface, not interacting with it, but striking a target, and sputter depositing a thin film on the substrate [100]. In this way the irradiation and deposition rates on the sample could be independently controlled. This work focused on how the composition of the target (GdCo, GdCo₂, or GdCo₃) affected the composition of the deposited film. Analysis was limited to modeling preferential sputtering rates and compositional analysis of the final result. Ar⁺ ion beams of 500-1000 eV were utilized, with an irradiating beam flux of ˜10¹⁴ ions/cm²/s on the substrate and a sputter deposition beam flux of ˜6.2*10¹¹ ions/cm²/s. This successfully demonstrated that the composition of the target could be used to control the composition of the final film, but did not take the opportunity to analyze surface morphology.

Motivation

The above sets the stage for the exploration of a potentially lucrative fabrication technique. Both ZnO and PDMS have important applications in a number of industries, including the biomedical market, with room to expand. One of the greatest challenges with this combination of dissimilar materials is that ZnO processing frequently requires temperatures far too high for PDMS. As stated earlier, ion beam processing is a viable option for room-temperature nanostructuring and oxidizing. This is critical to interface inorganic metals to polymers, such as PDMS. The advantages of interfacing these classes of materials is that it allows for novel materials that have both the properties of metal-oxide systems for sensors and polymer-based systems compatible with many practical environments, such as bio-systems. Some applications have gotten around this issue by utilizing multi-step processes, separating the ZnO preparation from its introduction to PDMS. Though successful, such increases in fabrication steps leads to additional time and cost to potential industrial-scale integration. Plasmas and ion beams are a natural choice as an alternative processing technique.

The following work aims to understand the behavior of the ZnO and PDMS system under simultaneous irradiation and codeposition in a dual ion beam setup. Based on conditions in the literature which have been shown to manipulate the morphology and chemistry of ZnO and PDMS separately, a number of systematic studies will be carried out to test the viability of this fabrication technique and to understand which parameters and mechanisms are responsible for the tunability of the final results. Specific ion beam parameters that will be explored include the ratio of fluxes between the two beams (and thus the relative deposition vs. irradiation), the energy of the ions, the total fluence that samples are exposed to, and the differences between inert and reactive ion beams on Si vs. PDMS. Overall, this holds the potential to develop a single-step process that will simultaneously deposit Zn, oxidize it to create ZnO, form nanostructures, and manipulate overall surface morphology and chemistry with high fidelity to meet the needs of biomaterial applications.

Facilities: Dual Ion-beam eXperiment (DIX) and Ion-Gas-Neutral Interactions with Surfaces (IGNIS)

The majority of experiments carried out herein were performed in two facilities at the Radiation Surface Science and Engineering Laboratory at the University of Illinois at Urbana-Champaign. Both vacuum facilities were custom designed to accommodate unique experimental setups requiring multiple ion beam modification sources, including the setup presented here.

Early work was carried out in the Dual Ion beam eXperiment (DIX). The purpose of this facility is to accommodate multiple surface modification and deposition tools, including broad and focused ion beams, magnetron sputtering guns, and evaporators, with a focus on highly specialized angles and geometries. The port configuration specifically allows for broad ion beams to be mounted perpendicular to each other, necessary for this work, or at projected perpendicular angles. Ion beam currents are measured with two electrically isolated circular titanium disks mounted perpendicular to each other to directly face both ion beams. The current plates are mounted on a manipulator, allowing for readings to be taken at chamber super center, and then removed when the sample is put in place for experiments.

Later work was carried out in the Ion-Gas-Neutral Interactions with Surfaces (IGNIS) facility. This features a much more robust design capable of in-situ, in-operando material modification and characterization. The centerpiece of this facility is the Specs PHOIBOS 150 NAP hemispherical analyzer, allowing techniques such as X-ray Photoelectron Spectroscopy (XPS) to be performed at pressures up to 5 mTorr. IGNIS is also designed to accommodate both forward and back scattering ion-scattering spectroscopy, Raman spectroscopy, ellipsometry, Secondary Neutral Mass Spectroscopy (SNMS), as well as several deposition sources. The manipulator can heat the sample to 900° C., cool to <190° C. with liquid nitrogen, electrically bias the sample, and move the sample with five degrees of motion. A separate small chamber is attached to IGNIS, known as the load lock, which serves as the loading and unloading area for samples. It has a much smaller volume than the main chamber and has its own dedicated pumping system. This allows it to pump down to UHV pressure relatively quickly after loading a sample, which can then be transferred to the manipulator in the main IGNIS chamber.

The underside of the manipulator is equipped with two titanium plates to measure ion beam current. The outer plate is grounded to the chamber and has a circular hole to let a specified cross sectional area of the beam reach a secondary plate. This plate is electrically isolated from the rest of the system and connected to an ammeter. The current and area can then be used to calculate the ion beam flux.

At the time this work was performed not all systems within IGNIS were online. Manipulator motion, current plate, and dual ion beam sources were utilized. Both broad ion beam sources used are the GenII Plasma Source manufactured by Tectra GmbH Physikalische Instrumente in Frankfurt, Germany. These sources ignite a plasma within a ceramic cup via microwaves generated in a magnetron and are facilitated by the electron cyclotron resonance effect [101]. A pair of molybdenum grids is then electrically biased to extract an ion beam of 0-2000 eV approximately 2.5 cm in diameter.

The GenII Plasma Source is installed on a vacuum chamber. At the end of the source, are the grids that extract and accelerate the ion beam. The sample is normal to the ion beam, and the current plate is facing away from the beam during the experiment.

2.2 Codeposition and Irradiation Setup and Procedures

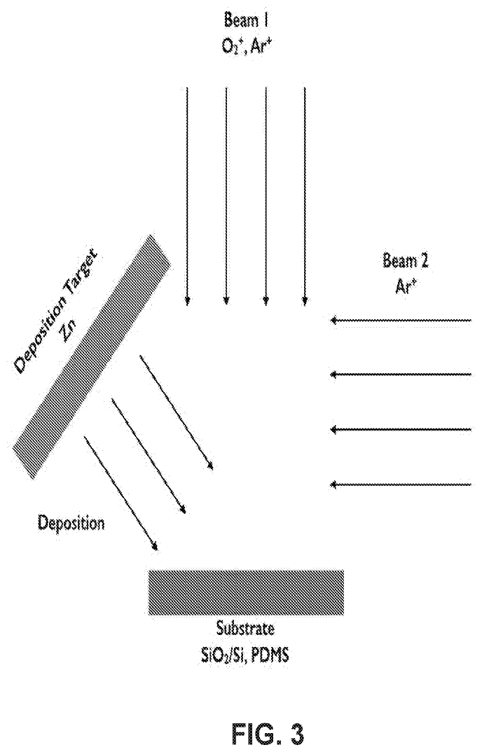

Two GenII Plasma Sources are mounted perpendicular to each other to facilitate the experimental setup. Samples are mounted on a custom-built stage, as shown below in FIG. 3. Four polycrystalline zinc targets are mounted at a 45° angle. Beam 1 impinges on the surface at normal incidence and does not interact with the Zn deposition source. Beam 2 impinges on the Zn target at 45°, sputtering Zn that is then deposited on the substrate. It is key that Beam 2 passes over, but does not interact with the substrate. In this way the two processes, material deposition and sample irradiation, are independently controlled through a number of factors including ion species and flux.

Several systematic studies were performed utilizing this setup. One of the primary factors explored is the ratio of fluxes between the two beams. Since exposures were carried out simultaneously, the ratio is the same for both flux and fluence, and is defined as follows,

$$\prod = \frac{{φ}_{1}}{{φ}_{2}} = \frac{{φ}_{1}}{{φ}_{2}}$$

where ϕ₁ and ϕ₂ are the fluxes of Beam 1 and Beam 2, respectively, and Φ₁ and Φ₂ are the fluences of Beam 1 and Beam 2, respectively. Since the deposition flux is proportional to that of Beam 2, this parameter indicates the relative deposition vs. sputtering rate from the substrate surface. Additional factors include substrate material (SiO₂ and PDMS), irradiation species (O₂⁺ and Ar⁺) to compare chemical and ballistic effects, ion energy, and total fluence, which are detailed below.

Sample Preparation and Handling

Two sample materials were utilized in this work, Si, with a thin naturally oxidized SiO₂ layer at the surface, and PDMS. Due to the significant differences in these materials, separate preparation and handling procedures was required.

The Si(100) samples were purchased as a single 3″ wafer with one side polished. The backside was scored with a diamond tip scribe parallel to the small flat edge to ensure breakage along a plane. The wafer was then placed between two glass microscope slides wrapped in Kimwipes delicate task wipers to minimize contact between hard surfaces. With the scored line placed at the edge of the slides, the protruding piece of wafer was tapped until it broke off. This process was repeated, changing Kimwipes between each step for cleanliness, until individual samples ˜1×1 cm were produced. These were then cleaned in an ultrasonic bath of acetone for 30 minutes, then rinsed with isopropyl alcohol and visually inspected for debris or scratches. They were then placed face down in individual rounded sample holders to prevent contact with the surface, with a spring to keep them in place. Following this, samples were only handled with vacuum clean tweezers. For experiments and certain analysis techniques, samples were mounted on various stages using carbon tape.

The PDMS samples were prepared using a Dow Corning Sylgard® 184 Silicone Elastomer Kit. A liquid base and curing agent were weighed with a chemical scale and mixed in a 10:1 ratio thoroughly with a spatula for 5 minutes. This mixture was then poured into a ceramic dish to a depth of ˜1 mm. This was then placed on a hot plate and heated to 150° C. for 10 minutes, then allowed to cool. After cooling for ˜1 hour, a razor blade was used to divide the PDMS into ˜1×1 cm samples and removed from the dish. Due to the flexible nature of PDMS, these samples were again placed in individual sample holders, but this time face up without a spring to ensure the surfaces were not disturbed. PDMS does not adhere well to carbon tape or copper tape and therefore the samples were always kept upright while in sample holders, during experiments, and during analysis.

Data Collection and Error Analysis

Experiments began by mounting a SiO₂ and PDMS sample side-by-side on the codeposition setup. The load lock is then isolated with gate valves and vented to atmospheric pressure with Ar. A door is unlocked and positive pressure within the load lock minimizes the amount of atmosphere and water vapor that can enter during sample loading. Once the samples are loaded onto the transfer arm the door is closed, gas flow turned off, and pumping begins. First, a scroll pump brings the load lock down to ˜3E-3 Torr, after which it is closed off and the turbomolecular pump opened. Once the load lock is pumped down below 1E-7 Torr, the gate valve separating the load lock from the main chamber is opened. Due to the constraint that the PDMS remain upright, the rest of the experiment is setup before transferring the samples to the manipulator.

Once the pressure between the chambers has stabilized at approximately 5E-8 Torr, this is recorded as the base pressure and the startup procedure for the ion sources is initiated. The manipulator is adjusted so that the current plate is sitting at chamber supercenter (the point where the sample will sit) and facing Beam 1. First, the gas flow to Beam 1 is turned on and the chamber pressure is raised to ˜4E-4 Torr, then the gas flow to Beam 2 brings the total to ˜8E-4 Torr. This is slightly higher than normal operation, but necessary to ensure both sources have adequate gas flow. The magnetron power supply to Beam 1 is then turned on and the power slowly increased until plasma is struck. This is indicated both by a visible glow within the chamber and a slight current reading on the ammeter as stray electrons are collected. This is then repeated for Beam 2. At this point plasma is ignited in the ceramic cup of both ion sources but no beam is being extracted. On the grid supply to Beam 1, the extraction voltage is set to −400 V, and then the energy is set depending on the experimental parameters. The extraction voltage is then adjusted slightly to minimize the current on both grids, as this will cause sputtering and wear them down over time. The magnetron power is then adjusted to produce a stable current as read on the ammeter. This can take up to 5 minutes as the source heats up and reaches equilibrium. At this point the power supply controlling the extraction and acceleration grids is shut off with the settings still in place, effectively turning of the ion beam but leaving the plasma. The current plate is then tilted toward Beam 2 and the procedure is repeated until a stable beam is established. The current plate is then moved back and forth as the source beams are turned on and off and adjusted to reach the desired flux ratio. This is determined by recording the beam current in a spreadsheet designed to calculate the flux. Once both ion beams reach the desired setting they are both shut off and the sample is transferred to the manipulator. A timer is set based on the calculated flux and the desired fluence and both ion beams are turned on simultaneously.

Over long periods of time (>10 minutes) the ion beam current can slowly drop. Testing has shown that this occurs with both Ar⁺ and O₂⁺. While the experiment is running, the current plate is not facing either of the ion beams, but does collect stray charged particles. This results in a reading lower than but proportional to the actual current. This is monitored during the experiment to maintain beam flux. Since the effect is expected from both sources, the power of both is slightly adjusted to return the current to its initial value. Since the quantitative change in current cannot be measured in-operando, the deviation from the initial value is kept within 10%, introducing error into the fluence. At the end of the experiment, the ion beams are shut off, the samples are transferred back into the load lock, and the beam currents are individually checked to ensure they are close to their original values. With the load lock once again separated via gate valve, the magnetron power supplies are then slowly shut off to prevent thermal shock to the internal ceramics and gas flow is turned off. The load lock gate valves are then shut, the load lock is vented with Ar, the door opened, and the samples removed and returned to their sample holders.

Several ex-situ analysis techniques were used on samples following codeposition, including atomic force microscopy (AFM), x-ray photoelectron spectroscopy (XPS), and contact angle. These were all carried out at the Frederick Seitz Materials Research Laboratory at the University of Illinois Urbana-Champaign. AFM was performed with the Asylum Research Cypher instrument, which offers extremely high-resolution capabilities. Imaging was performed in tapping mode, producing height, amplitude, and phase scans. The majority of images presented are color map height images. Although these contain the most meaningful physical representation of the surface, with features being shown with length scale, the lateral resolution across the surface can sometimes suffer from the electronic feedback loop of the instrument. The amplitude and phase scans, meanwhile, represent the deflection and oscillation phase offset of the probe tip. These are measured as a voltage and degree that are not a direct measurement of the surface, however they are recorded in real time and thus have greater lateral resolution. In some cases the amplitude data is included as a supplement to the height data to give a better representation of what the surface looks like, or the outline of surface features. AFM data was analyzed with both Asylum Research's ARgyle™ and WSXM software [102].

XPS data was taken with the Physical Electronics PHI 5400 instrument, utilizing Al K-α x-rays from a monochromatic x-ray source. CasaXPS software was used to analyze the data. First, survey scans were performed with a pass energy of 160 eV. Based on this, core scans were then performed over identified peaks with a pass energy of 40 eV. For each core scan there were 10 sweeps that were averaged. Based on these, the area of the peaks associated with each element was then adjusted with a sensitivity factor to calculate atomic concentration (%) of different elements on the surface. Wettability testing was performed with contact angle measurements with a ramé-hart goniometer. Droplets of water were placed at several locations on the sample surface and photographed. These images were then analyzed to determine the average contact angle between the surface and the water droplet. High contact angles indicate hydrophobic samples, while low contact angles indicate hydrophilic ones.

Error is present and propagates throughout the measurements and calculations in this work. The incident angle of irradiation from the ion beam was directly measured on the sample manipulator and the error is related to the smallest degree on the gauge, 1°, therefore the error is ±1°.

σ_{θ}=±1°

Another important setting for these experiments is the ion energy. This, however, is set on the power supply and not directly measured. Additionally, no data on this is listed on the equipment or in the manual. The supply displays energy in the range of 0-2 keV with 250 eV marked increments.

The most significant error calculation is that for the fluence the sample is exposed to. This error propagates from both the angle of incidence and the area of the current collection plate to the flux, and finally the fluence. The area of the current plate was calculated from the measured diameter, with error originating in this measurement. The bias error inherent in the instrument is ±0.01 mm. The precision error from multiple measurements also results in ±0.01 mm, resulting in the following.

total=\sqrt{precision²+bias²}=\sqrt{(0.01 mm)²+(0.01 mm)²}=0.01414 mm

d=11.07±0.01 mm

The error is first shown in full, and then represented by the correct number of significant figures. This is then converted into the radius.

$$r = \frac{d}{2} = \frac{11.07 \pm 0.01 mm}{2} = 5.54 \pm 0.01 mm$$

The radius is then used to calculate the area of the plate.

A=πr²=96.4 mm²

which yields the following error for the calculation of area.

$${σ}_{A} = A * 2 * \frac{{σ}_{r}}{r} = 96.42 {mm}^{2} * 2 * \frac{0.01 mm}{5.54 mm} = 0.35 {cm}^{2}$$

This gives a final area of 96.42±0.35 mm².

The final directly measured value that contributes error to the flux calculation is the directly measured current. Though the ammeter used to measure this current is highly accurate (quote value), the stability of the gun causes this to waver over time. As explained above, by monitoring the proportional current during the experiment, the current was kept within 10% of the initial value, so this contributes ±10% to the bias error. A second factor, secondary electron emissions, also contributes to the bias error. Incident ions on the current plate will tend to eject electrons from the current plate, which will also contribute to error in the current reading. Published work on this shows that 1 keV Ar⁺ at normal incidence on a wide variety of metals results in an extremely small secondary electron emission coefficient (γ) [103,104]. A very conservative value of 10% will be used here. This leads to the following calculation for error in the current.

σ_{I}=\sqrt{(0.1*I)²+(0.1*I)²}=0.1414*I

The derivation of flux is then as follows, beginning with the measured current and assuming an ion beam of single-charged ions.

$$I = \frac{charge}{time} \to [ A ] = \frac{[ C ]}{[ s ]} * \frac{1 [ ion ]}{1.602 * 1 {0}^{- 19} [ C ]} = 6.242 * 1 {0}^{18} [ \frac{ion}{s} ]$$

This describes the total current. The flux can then be described as this current over the area in which the current is collected, to yield ions/cm²/s.

$$φ = \frac{I}{A} \to \frac{6.242 * 1 {0}^{18} [ \frac{ion}{s} ]}{0.99 [ {cm}^{2} ]} = 6.305 * 1 {0}^{18} [ \frac{ion}{{cm}^{2} * s} ]$$

This, however, is only accurate if the incident flux is normal to the sample surface. Correction for this requires a factor of cos(ϑ). This results in the following flux equation.

$$φ = I * 6. 305 * 1 {0}^{18} [ \frac{ion}{{cm}^{2} * s} ] * cos ( θ )$$

or, symbolically

$$φ = \frac{I * cos ( θ )}{A}$$

The error in flux can then be expressed as follows.

$${σ}_{φ} = \sqrt{{( \frac{\partial φ}{\partial I} {σ}_{I} )}^{2} + {( \frac{\partial φ}{\partial θ} {σ}_{θ} )}^{2} + {( \frac{\partial φ}{\partial A} {σ}_{A} )}^{2}} \\ {σ}_{φ} = \sqrt{{( \frac{cos ( θ )}{A} {σ}_{I} )}^{2} + {( \frac{- I * sin ( θ )}{A} {σ}_{θ} )}^{2} + {( \frac{- I * cos ( θ )}{{A}^{2}} {σ}_{A} )}^{2}}$$

The ratio of two flux measurements is then described by the following.

$$\prod = \frac{{φ}_{A}}{{φ}_{B}}$$

The error propagates in this calculation as follows.

$${( \frac{{σ}_{\prod }}{\prod } )}^{2} = {( \frac{{σ}_{{φ}_{A}}}{{φ}_{A}} )}^{2} + {( \frac{{σ}_{{φ}_{B}}}{{φ}_{B}} )}^{2}$$

$${σ}_{\prod } = \prod * \sqrt{{( \frac{{σ}_{{φ}_{A}}}{{φ}_{A}} )}^{2} + {( \frac{{σ}_{{φ}_{B}}}{{φ}_{B}} )}^{2}}$$

Additionally, the fluence is then calculated from the flux with time.

Φ=ϕ*t

Or, conversely the required experimental time can determined from a desired fluence and measured flux.

$$t = \frac{Φ}{φ}$$

$${σ}_{t} = \sqrt{{( - \frac{φ}{{φ}^{2}} * {σ}_{φ} )}^{2}}$$

The time of the experiment is monitored with a stopwatch with an assumed error of ±1 s. The error in fluence is then as follows.

$${σ}_{Φ} = Φ * \sqrt{{( \frac{{σ}_{φ}}{φ} )}^{2} + {( \frac{{σ}_{t}}{c} )}^{2}}$$

There are several measurements that were repeated a number of times, including the diameter and height of surface features. The presented value is the average and the error is calculated in the following manner.

$${σ}_{\overline{x}} = \frac{{σ}_{x}}{\sqrt{n}}$$

Simulation of Ion Beam Sputtering Deposition

A key goal of the codeposition fabrication approach is to produce individual surface features. This makes a low deposition rate desirable to prevent complete coverage of the surface. Due to the nature of this setup, there is simultaneous deposition and sputtering from the sample surface. The following is an estimate of the deposition of Zn throughout the experiment. The Stopping and Range of Ions in Matter (SRIM) software package was utilized to simulate the sputtering of Zn via Ar⁺ ions as near as the possible. SRIM utilizes Binary Collision Approximation (BCA) to simulate ions impacting surfaces and calculate the resulting collision cascade, dpa, sputter yield, and sputtered atom energy, among other things. The approach has several limitations, however, with the most significant to this application being the lack of ability to account for chemical effects [105]. Since O₂⁺ irradiation, leading to oxidation, is a central theme of this work, the following results should be taken as rough estimates intended to establish the context of the experiments and not precise quantitative results.

A 500 Å thick Zn surface was simulated to be bombarded at 45° by Ar⁺ ions at 500 eV and 1000 eV for 100,000 runs. SRIM calculated the sputter yield to be 6.47 atoms/ion at 500 eV and 10.38 atoms/ion at 1000 eV.

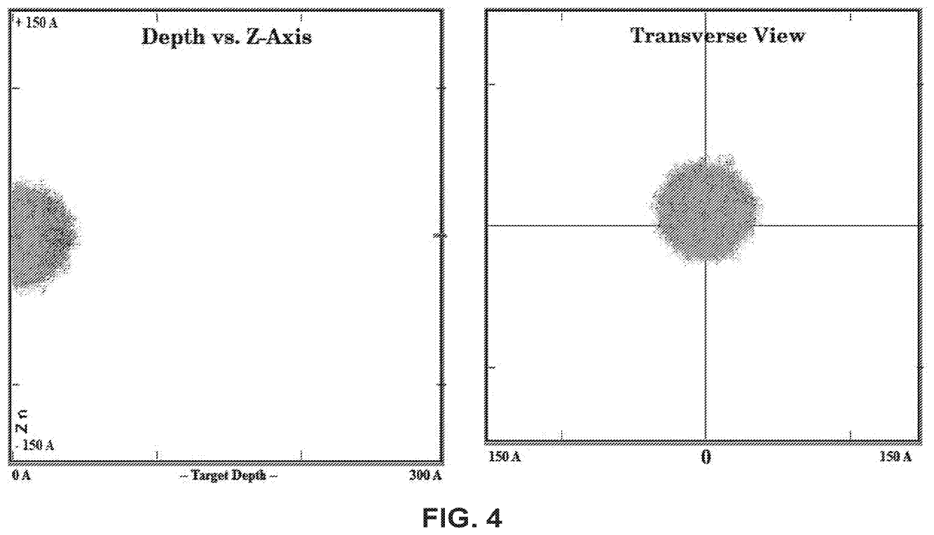

FIG. 4 graphically shows the results of the SRIM simulations for the 500 eV Ar⁺ case. The image on the left depicts the collision cascade via a cross-section of the sample, with the left edge representing the Zn surface, and moving deeper toward the right. This graphically shows the calculated radial ion range of 13 Å. The image on the right shows a top-down view of the same with a lateral projection range of 10 Å. It can be seen from this perspective that the damage cascade is skewed toward the top of the image. This is due to the 45° incident angle.

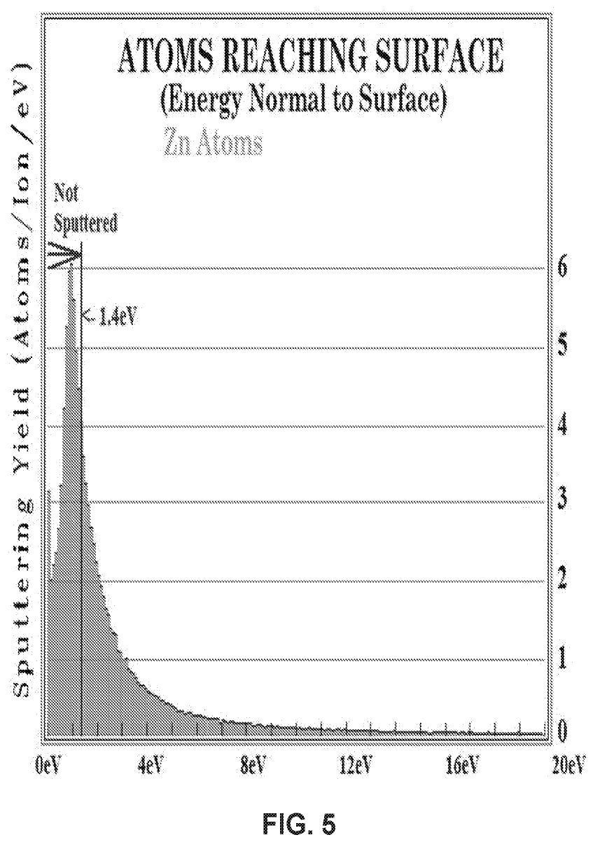

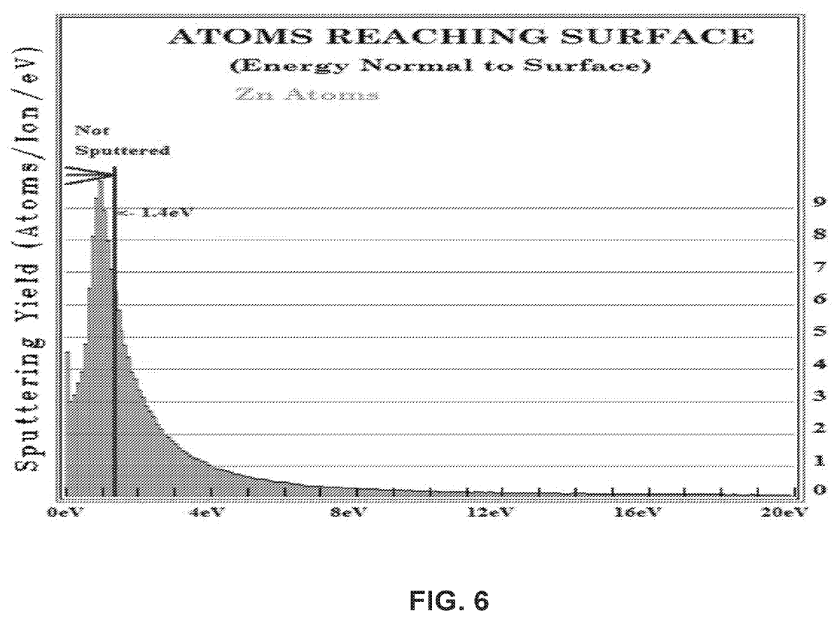

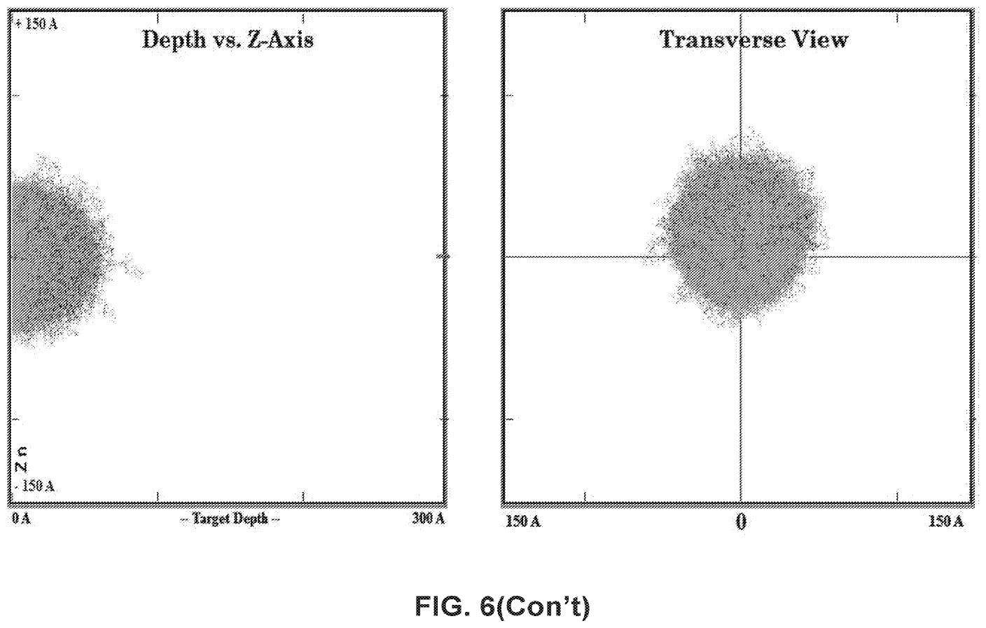

FIG. 5 shows the energy distribution of Zn surface atoms impacted by the 500 eV Ar⁺ beam. This is shown to follow a Thompson distribution, where those atoms below the surface binding energy of 1.4 eV are not sputtered. Thus, the distribution of energy among Zn atoms deposited on a substrate from this sputtering is indicated by the area to the right of the vertical line, ignoring any collisions that may occur between sputtering and deposition. This was then repeated for the 1000 eV sputtering case, and is shown in FIG. 6, right, top and bottom.

FIG. 5, right, graphically shows the results of the SRIM simulations for the 1000 eV Ar⁺ case. The image on the top right depicts the collision cascade via a cross-section of the sample, with the bottom right again representing the Zn surface, and moving deeper toward the right. This graphically shows the calculated radial ion range of 18 Å, which can be seen by the noticeably larger cascade. The image on the right shows a top-down view of the same. Once again, there is pronounced shift of the cascade away from center due to the 45° incident angle, with a lateral projection range of 14 Å. This data can then be used to estimate the deposition of Zn via sputtering.

FIG. 6 graphically shows the results of the SRIM simulations for the 1000 eV Ar⁺ case. The image on the right, top depicts the collision cascade via a cross-section of the sample, with the left edge again representing the Zn surface, and moving deeper toward the right. This graphically shows the calculated radial ion range of 18 Å, which can be seen by the noticeably larger cascade. The image on the right, bottom shows a top-down view of the same. Once again, there is pronounced shift of the cascade away from center due to the 45° incident angle, with a lateral projection range of 14 Å. This data can then be used to estimate the deposition of Zn via sputtering. FIG. 6, left, shows the energy distribution of Zn surface atoms impacted by the 1000 eV Ar⁺ beam. This again is shown to follow a Thompson distribution, where those atoms below the surface binding energy of 1.4 eV are not sputtered. Compared to FIG. 5, the increased sputter yield between 1000 eV ions and 500 eV ions can be seen. The distribution of energy among Zn atoms deposited on a substrate from this sputtering, then, is indicated by the area to the right of the vertical line, ignoring any collisions that may occur between sputtering and deposition.

First, the range of the total number of sputtered atoms will be calculated at each energy level. At 500 eV, sputter fluences ranged from 4.95E16 ions/cm² to 1.00E18 ions/cm². Given a Zn target surface area of 4 cm² and a sputter yield of 6.47 atoms/ion, this results in a total number of sputtered Zn atoms of 1.28E18-2.59E19 atoms. Assuming the (0001) plane of the HCP Zn matrix, the planar density can be calculated as follows [54].

First, the range of the total number of sputtered atoms will be calculated at each energy level. At 500 eV, sputter fluences ranged from 4.95E16 ions/cm² to 1.00E18 ions/cm². Given a Zn target surface area of 4 cm² and a sputter yield of 6.47 atoms/ion, this results in a total number of sputtered Zn atoms of 1.28E18-2.59E19 atoms. Assuming the (0001) plane of the HCP Zn matrix, the planar density can be calculated as follows [54].

$$PD = \frac{1}{2 {R}^{2} \sqrt{3}} = \frac{1}{2 {( 0.1 \; 33 nm )}^{2} \sqrt{3}} = 16.27 atoms \text{/} {nm}^{2}$$

Given this, and a sample surface area of 2 cm², a film thickness of 0.195-3.937 μm would be deposited if all of the Zn made it to the sample and none were removed from the irradiating ion beam. Due to the solid angle between the sputtering source and the sample surface, an estimated 10% of the sputtered atoms make it to the sample, resulting in an estimated deposition of 19.5-393.4 nm Zn for 500 eV sputtering.

Similarly, at 1000 eV, sputter fluences ranged from 5.05E16 ions/cm² to 1.00E18 ions/cm². Given the same Zn target area of 4 cm² and a sputter yield of 10.38 atoms/ion, this results in a total number of sputtered Zn atoms of 2.097E18-4.152E19 atoms. Utilizing the same planar density as above, and again assuming complete deposition and ignoring subsequent sputtering, this results in a film thickness of 0.319-6.312 μm. With the same fraction of atoms being deposited on the sample, a final estimated deposition of 31.9-631.4 nm Zn is achieved at 1000 eV sputtering. It is worth noting that in the experimental setup, the process described above often occurs in the presence of oxygen.

Considering that simultaneous sputtering of the sample with another ion beam occurs, a second set of SRIM simulations were performed. In these, 100,000 runs of O₂⁺ incident at 0° were performed on a 20 Å layer of Zn over a 500 Å layer of SiO₂. These results are shown below.

$$PD = \frac{1}{2 {R}^{2} \sqrt{3}} = \frac{1}{2 {( 0.1 \; 33 nm )}^{2} \sqrt{3}} = 16.27 atoms \text{/} {nm}^{2}$$

Given this, and a sample surface area of 2 cm², a film thickness of 0.195-3.937 μm would be deposited if all of the Zn made it to the sample and none were removed from the irradiating ion beam. Due to the solid angle between the sputtering source and the sample surface, an estimated 10% of the sputtered atoms make it to the sample, resulting in an estimated deposition of 19.5-393.4 nm Zn for 500 eV sputtering.

Similarly, at 1000 eV, sputter fluences ranged from 5.05E16 ions/cm² to 1.00E18 ions/cm². Given the same Zn target area of 4 cm² and a sputter yield of 10.38 atoms/ion, this results in a total number of sputtered Zn atoms of 2.097E18-4.152E19 atoms. Utilizing the same planar density as above, and again assuming complete deposition and ignoring subsequent sputtering, this results in a film thickness of 0.319-6.312 μm. With the same fraction of atoms being deposited on the sample, a final estimated deposition of 31.9-631.4 nm Zn is achieved at 1000 eV sputtering. It is worth noting that in the experimental setup, the process described above often occurs in the presence of oxygen.

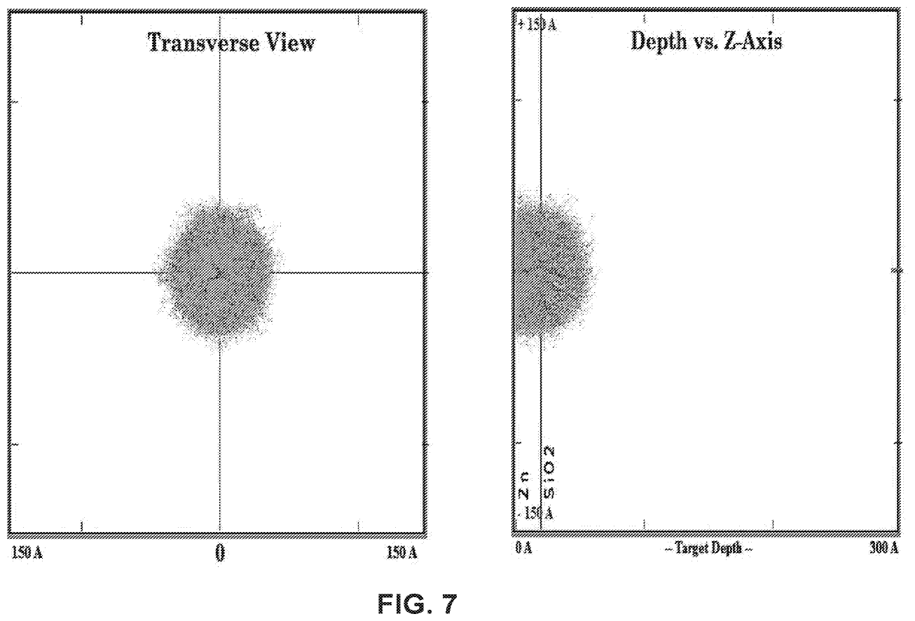

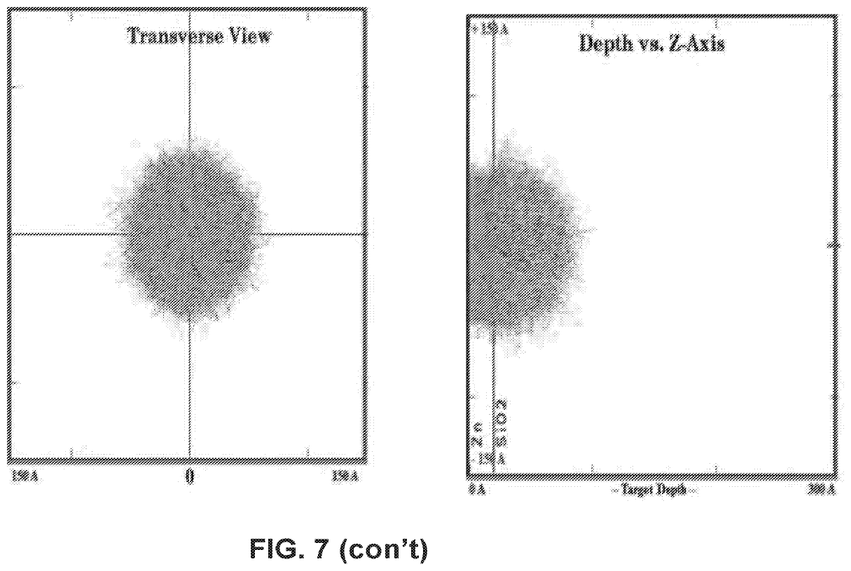

Considering that simultaneous sputtering of the sample with another ion beam occurs, a second set of SRIM simulations were performed. In these, 100,000 runs of O₂⁺ incident at 0° were performed on a 20 Å layer of Zn over a 500 Å layer of SiO₂. These results are shown in FIG. 7.

FIG. 7, left, graphically shows the results of the SRIM simulations for the 500 eV O₂⁺ case. These depict many similar features as FIGS. 5 and 6. The cross-sectional image on the left, top shows a side view of the damage cascade. It can be seen to expand slightly once the interface is reached due to the differences in material properties, such as density. This depicts an 11 Å radial ion range. The top-down view (left, bottom) again shows this, with one notable difference. In this case the damage cascade is centered instead of shifted upward due to the normal incident angle, as opposed to the previous 45°, with a lateral projection range of 7 Å. The similar case at 1000 eV is shown on the right, top and bottom. FIG. 7, right, top, graphically shows the results of the SRIM simulations for the 1000 eV O₂⁺ case. Once again, this is similar to the previous results, with slight variations due to the higher energy. Here, the radial ion range is 16 Å and the difference upon reaching the interface between Zn and SiO₂ is more pronounced. Again, the top-down view, right, bottom shows a centered damage cascade with a lateral projection range of 10 Å.

This produced sputter yields of 5.26 and 7.46 atoms/ion for 500 and 1000 eV O₂⁺ ions, respectively. Given a constant fluence of 1E17 ions/cm², on a solid Zn sample, this would remove a thickness of 80.02 nm at 500 eV and 113.5 nm at 1000 eV. In practice, the oxidation of the deposited material would result in a significantly lower sputter yield. The two above processes, deposition and sputtering, however, occur simultaneously. Given the sputtering rate is estimated to be less than the deposition rate, it is expected that ZnO (due to oxidation when the irradiating beam is O₂⁺) will be present on the surface.

Another significant detail to come out of these simulations is the energy of the sputtered Zn atoms. This is a parameter that is not well controlled in many deposition techniques and could play an important role in surface morphology evolution. The simulations yielded an average sputtered atom (Zn) energy of 10.36 eV/atom during 500 eV sputtering and 11.96 eV/atom during 1000 eV sputtering, with their distributions shown in FIGS. 5 and 6.

Codeposition Results on Si

The results of several experimental parameter sets from codeposition experiments on Si substrates are presented and discussed herein. While the focus of this work as a whole is concerned with the understanding and development of ion beam processing of ZnO with PDMS, polymer chemistry is highly complex on its own, and is further complicated by the intricate interaction with irradiation, and little work has previously been done that is relevant to this experimental setup. Si, however, is well understood in the context of this work and is part of the backbone of the PDMS polymer chain, making it a good candidate for comparison. The first parameter space in the codeposition and irradiation work focuses on the ratio between the irradiation and deposition ion beam fluxes. This is directly correlated with the relative deposition and sputtering rates of material from the sample surface. The next sample set analyzes the effects of ion energy, highlighting the similarities and differences between 500 and 1000 eV ions. Following that, results are presented for samples that were irradiated to a significantly greater fluence (5 times) to explore the nature of surface feature evolution and development. The chapter concludes with a summary of these results. The overall data will be organized, examined, and compared as appropriate to the parameter of focus in each section and, as such, some results appear more than once.

Ion Beam Flux Ratio

The experimental setup used in this work, as described above, explicitly enables the independent control over the irradiation of the sample surface and the deposition rate of Zn. One of the primary parameters that can be controlled in this way is the ratio of ion beam fluxes (and consequently the end fluence). The following data sets hold parameters such as sample irradiation fluence, ion species, and ion energy constant to make this comparison. Experiments were performed with flux ratios of 0.1, 0.2, 0.5, 1.0, and 2.0 ranging from lower to higher sample irradiation relative to Zn deposition.

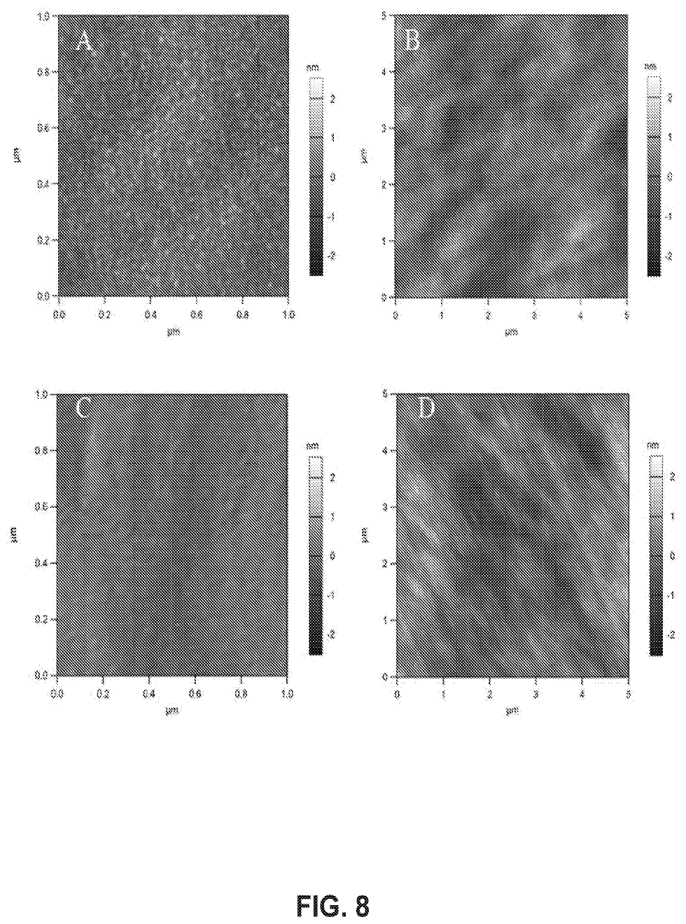

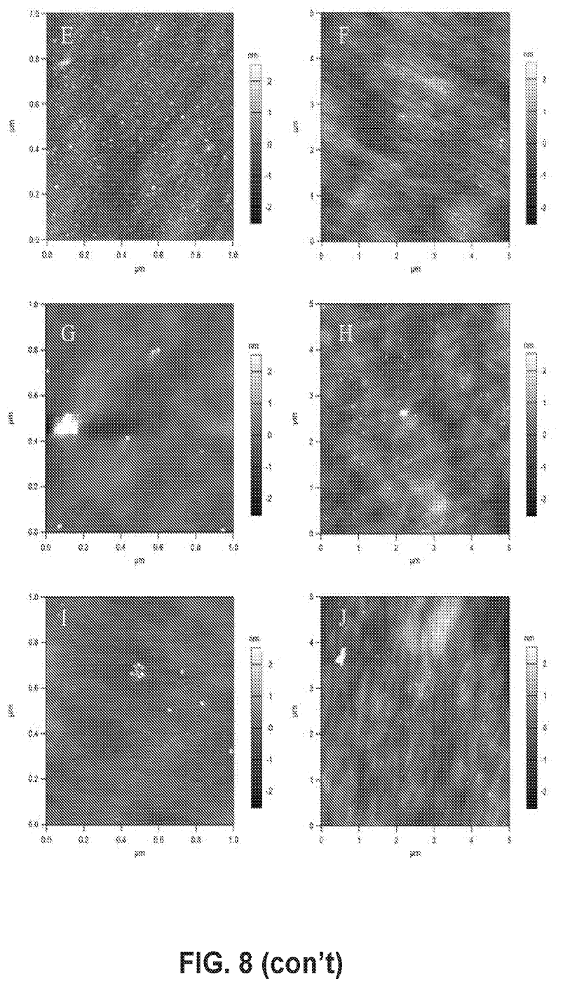

FIG. 8 shows AFM scans of Zn codeposition on Si, deposition with 500 eV Ar⁺, sample irradiated with 500 eV Ar⁺ to 1E17 ions/cm² with flux ratios of 0.1 (top; left, right), 0.2 (second from top, left, right), 0.5 (third from top, left, right), 1.0 (second from bottom, left, right), and 2.0 (bottom, left, right.) The first data set, shown in FIG. 8, shows the AFM height results of 500 eV Ar⁺ irradiation and codeposition over the aforementioned flux ratios, all with a final irradiation fluence of 1E17 ions/cm² on the Si substrate.

This data shows that the relative deposition rate has a significant effect on surface morphology. Images at top and second from top correspond to the highest deposition rates, with flux ratios of 0.1 and 0.2, and show no discernable surface features. Images at third from top, however, show a high density of small surface features <20 nm in diameter. At even high ratios, larger dots of ˜40 nm diameter are shown to form, but with lower areal density. Interestingly, very little change in roughness, as measured by Route Mean Square (RMS), was observed.

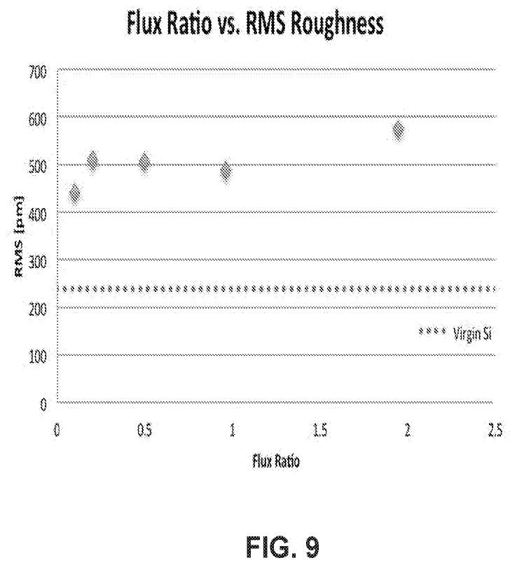

FIG. 9 shows that a slight increase in surface roughness is observed with increasing flux ratio, or decreased Zn deposition. Within this sample set, the highest deposition rate results in the smoothest sample surface, while the lowest Zn deposition resulted in the roughest. Additionally, no ordering of features is observed at any flux ratio under these conditions.

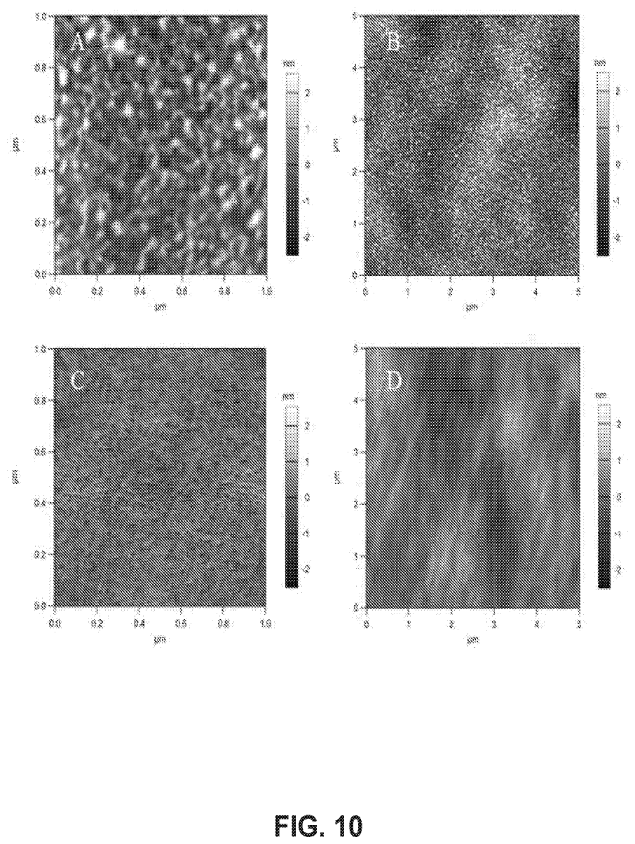

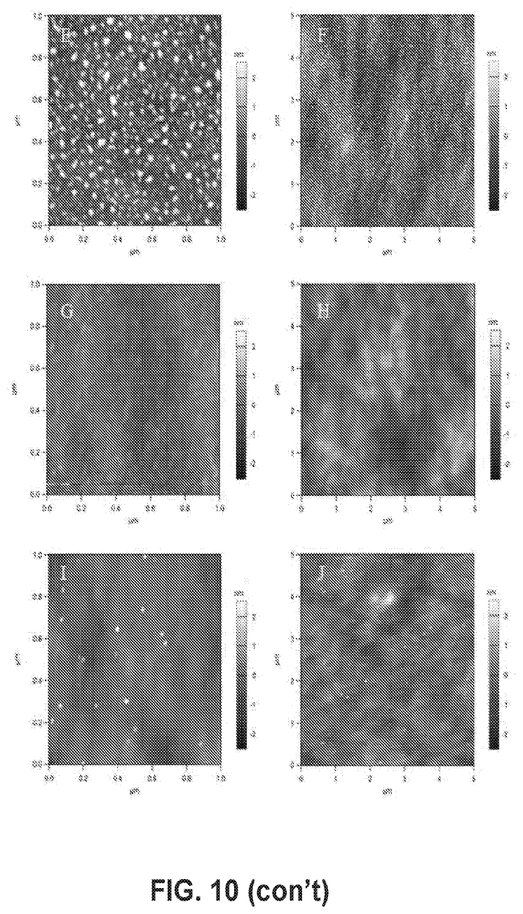

The previous experimental conditions were repeated on Si with an increase in ion energy from 500 eV to 1000 eV, with AFM results shown below in FIG. 10. FIG. 10 shows AFM scans of Zn codeposition on Si, deposition with 1000 eV Ar⁺, sample irradiated with 1000 eV Ar⁺ to 1E17 ions/cm2 with flux ratios of 0.1 (top, left, right), 0.2 (second from top, left, right), 0.5 (third from top, left, right), 1.0 (second from bottom, left, right), and 2.0 (bottom, left, right.)

This data set again shows that flux ratio has a significant effect on surface morphology. Under these conditions, the highest deposition rate does produce a notable change to the surface, with poorly defined dots being visible in images A-B. As before, significant feature growth is observed at a flux ratio of 0.5, with images E-F showing a high density of dots of 30-40 nm diameter. The lowest deposition sample with a ratio of 2.0 shows a much lower density of dots ˜20 nm in diameter. Greater variation in roughness is observed at 1000 eV than the 500 eV results shown previously.

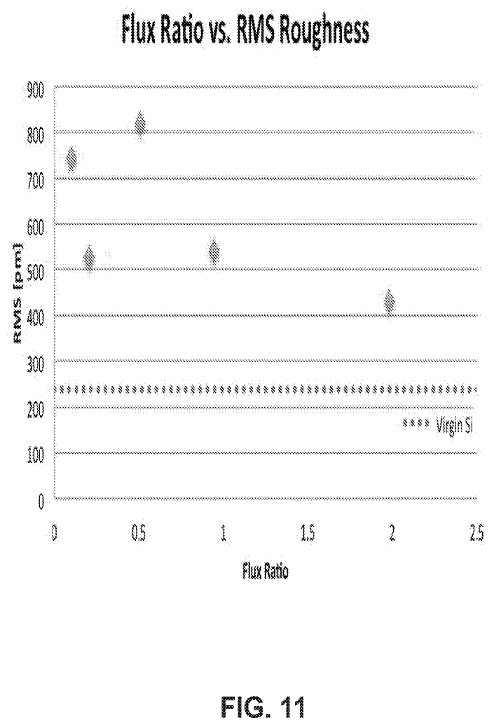

FIG. 11 shows the surface roughness variations with flux ratio for codeposition performed on Si with 1000 eV Ar⁺ ions. Flux ratios of 0.2, 1.0, and 2.0 show similar values to the 500 eV results. The higher deposition samples at flux ratios of 0.1 and 0.5, however, show notably rougher surfaces, though no clear correlation between roughness and flux ratio or deposition rate is observed. Again, no ordering of features occurs on any sample.

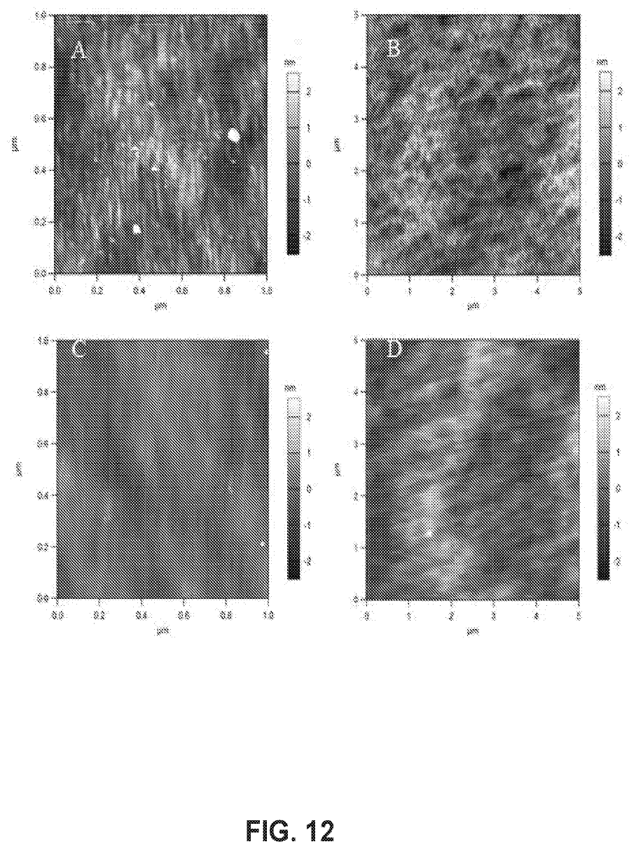

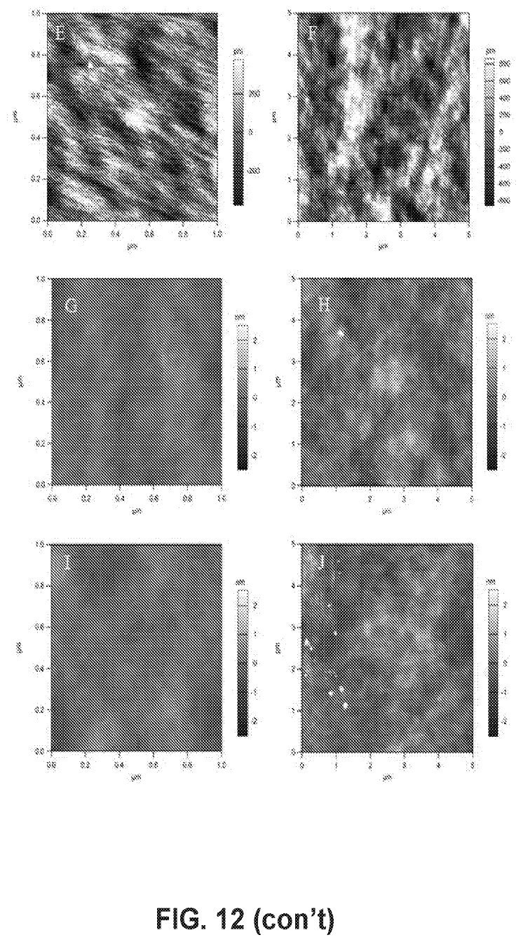

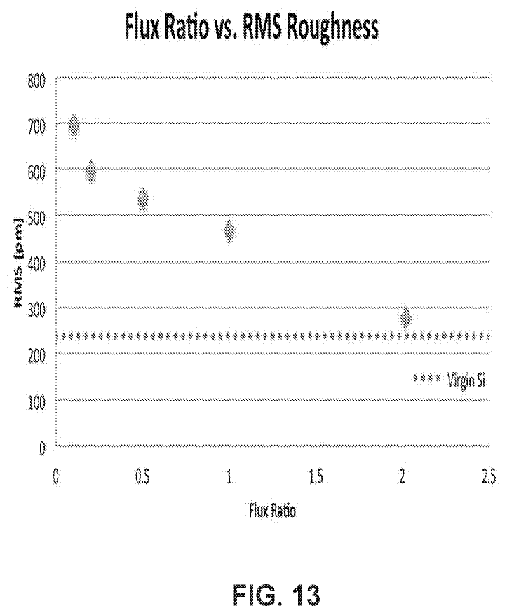

The above two experimental sets were then repeated with sample irradiation performed with O₂⁺ ions for the purpose of oxidizing the deposited Zn. The deposition ion beam impingent on the Zn target remained Ar⁺ to minimize the potential of continually oxidizing the surface and reducing the target sputter yield. The chemical effects of O₂⁺ compared to Ar⁺ are analyzed previously, while the surface morphology effects with flux ratio are shown in FIG. 12. This data shows far less dot formation than previous sets. The highest deposition rates show rough surfaces with a few dots forming as seen in images top and second from top. These also show what may be extremely early ripple formation, as can be seen in the vertical lines in images top left and second from top left. The highest density of dot is again seen at a flux ratio of 0.5 in images third from top, with similar features forming at lower density at a flux ratio of 1.0. Images second from bottom and bottom, flux ratio 2.0, show a few larger dots ˜130 nm in diameter surrounded by clusters of smaller features, though they appear to be grouped only in one area, with the rest of the sample being extremely smooth. This may have resulted from a physical or chemical surface defect and was not seen elsewhere on the sample. No ordering of features is observed at any flux ratio. Surface roughness data is shown in FIG. 13.

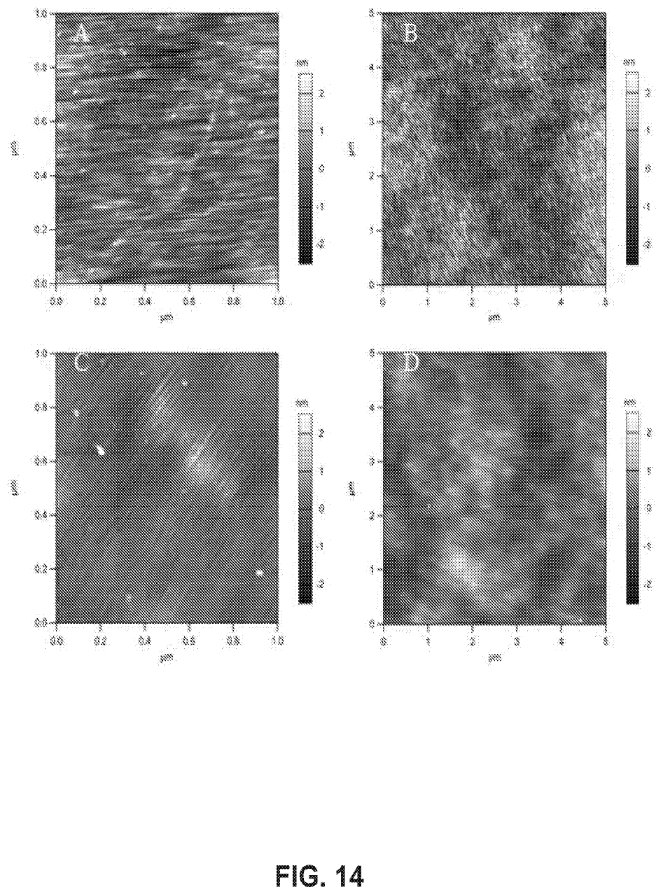

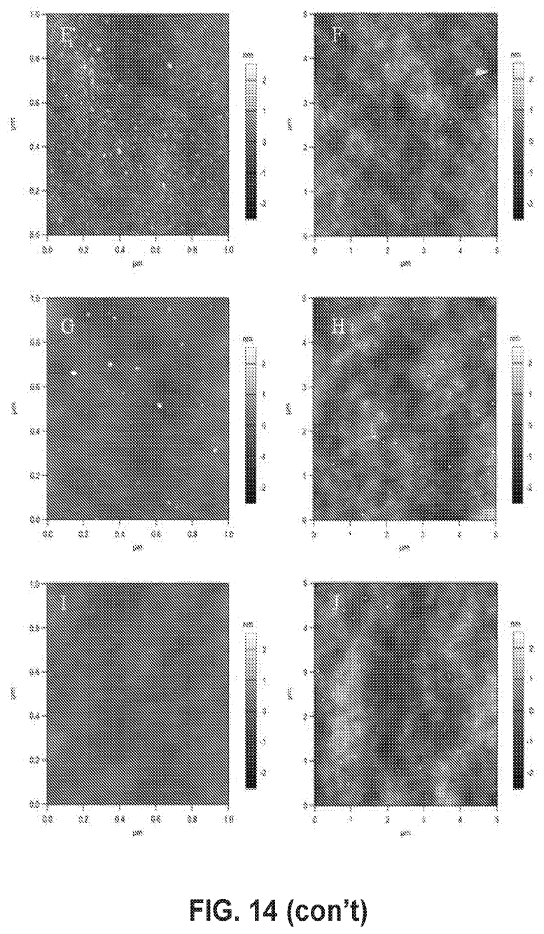

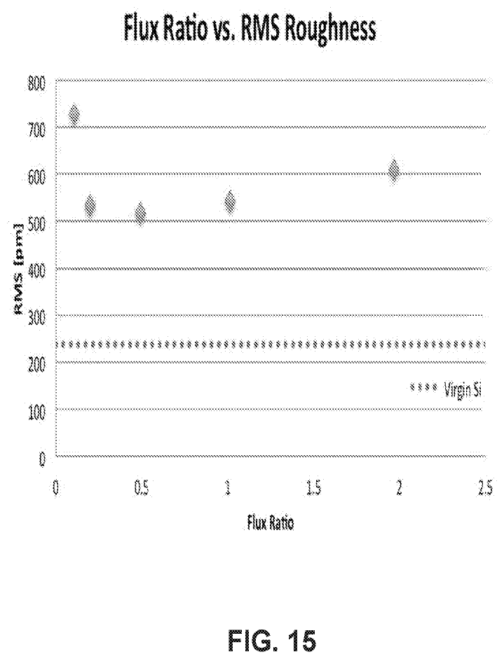

Codeposition of Zn on Si with 500 eV O₂⁺ irradiation shows a more clear relationship between surface roughness and the flux ratio. Here, surface roughness is shown to decrease with increasing flux ratio. Higher Zn deposition is shown here to result in rougher surfaces, regardless of clear feature formation, with increasingly smoother surfaces forming with decreased deposition. The next experimental set (FIG. 14-15) repeated the above conditions with O₂⁺ ion energy increased from 500 eV to 1000 eV.

FIG. 14 shows AFM scans of Zn codeposition on Si, deposition with 1000 eV Ar⁺, sample irradiated with 1000 eV O₂⁺ to 1E17 ions/cm² with flux ratios of 0.1 (top, left, right), 0.2 (second from top, left, right), 0.5 (third from top, left, right), 1.0 (second from bottom, left, right), and 2.0 (bottom, left, right). The increase in energy reveals more clear feature formation over flux ratios compared to the previous 500 eV O₂⁺ set. Image top left shows what appear to be precursors or early stage dots that are visible, but are very small and not clearly defined. Moving from a ratio of 0.1 to 0.2, more defined dots have formed but are very low in density. Both samples also show ripple formation with wavelengths ˜20 nm. Next, at a ratio of 0.5, images show a much higher density. At a ratio of 1.0, a lower density of more developed dots is seen, with the density dropping even further at a ratio of 2.0. This can also be seen in the surface roughness data. As seen before, the highest Zn deposition results in the roughest sample (FIG. 15). As the flux ratio is increased toward 0.5 where a high density of features is observed, the samples are continually smoother. Past this point, as the features become less dense but better defined, the roughness slowly increases.

Several clear trends can be seen in the above data sets that highlight the effects of the deposition and irradiation ion flux ratios. First, at the lower flux ratios (and consequently higher deposition rate within each data set), very few or no individual surface features are seen to form. This could be a result of high enough deposition that a Zn film fully covers the Si surface. At higher flux ratios, there is relatively lower Zn deposition and individual dots are shown to form. These are believed to result from the coalescence of the deposited Zn, driven by the energy imparted by the irradiating ion beam. The drop in density at higher ratios would then be a result of less Zn being present. Among all four sets that analyzed the effects of flux ratio, surface morphology appears to shift dramatically at a ratio of 0.5 from a randomly rough surface to one that shows clear dot formation. This will be further addressed in next experiments, which analyzes the chemical composition of the surface.

Energy Effects on Surface Morphology

The energy of ions incident on a material surface is another parameter that is both easily controlled and commonly used to tune modification methods. Increasing ion energy not only increases the total energy deposited on a surface, but also the ion penetration depth and energy distribution. For the codeposition setup, this affects both Zn deposition and substrate sputtering as shown in the simulations in chemical analysis and wettability results. Notable differences in ion beam flux ratio are thus highlighted below as related to the ion energy.

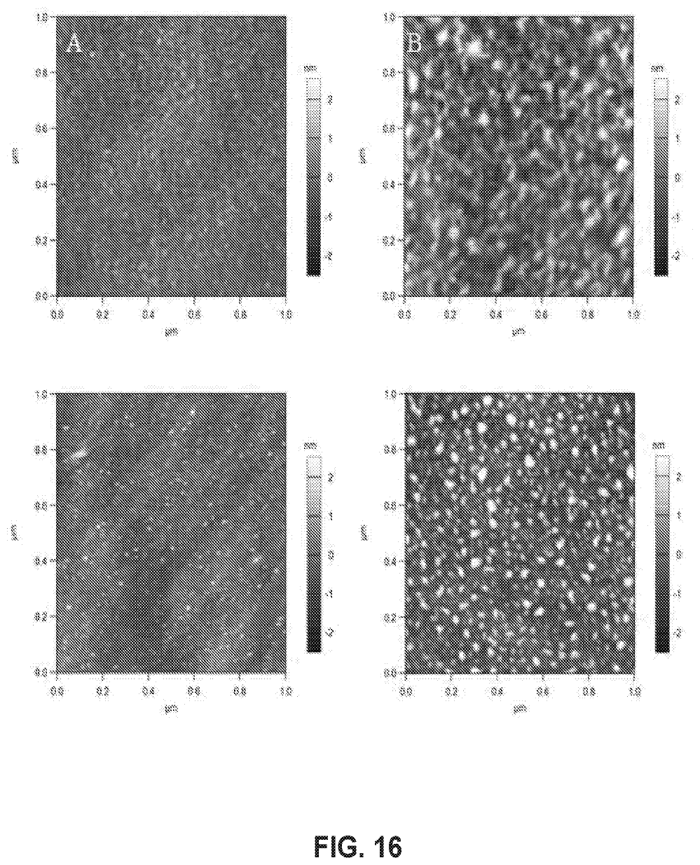

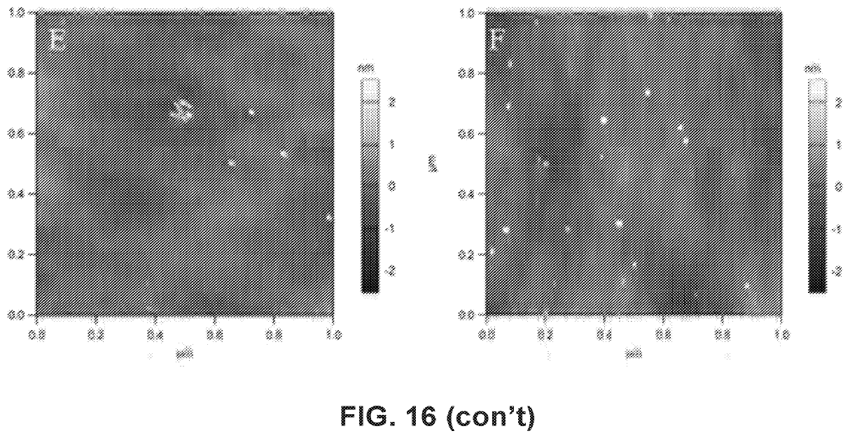

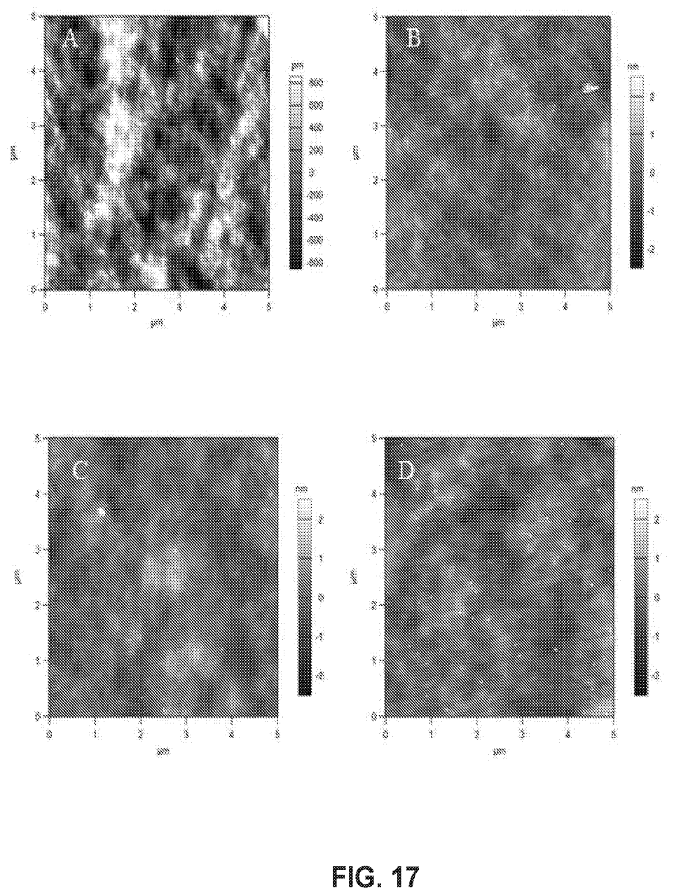

First, samples were irradiated with Ar⁺ at 500 eV and 1000 eV at a number of flux ratios. Those exposed to 1000 eV ions generally resulted in a higher density of larger, more clearly defined features compared to those exposed to 500 eV ions. This can be clearly seen at flux ratios of 0.1, 0.5, and 2.0. Images on the left in FIG. 16 show the 500 eV samples and those on the right the 1000 eV samples. The lowest flux ratio samples, top left and right, show very similar surface morphology, but with greater height variation at 1000 eV. This is again seen at a ratio of 0.5 in images second from top, left and right. Finally, images bottom left and right show that at the highest flux ratio the same features form, but at a much higher density at 1000 eV. Similar results can be seen by comparing the samples irradiated with 500 eV and 1000 eV O₂⁺. This is most clear with the 0.5 and 1.0 flux ratios. (FIG. 17). The effects of increasing energy on the size and density of dots created on the Si surface include an increase of both feature size and density. This could be a result of greater Zn deposition from a greater target sputter yield, though this is not straightforward since the increase in ion energy also increases the sputter yield from the sample surface, effectively increasing both the deposition and removal rates. The simulations above address this issue. The modeled interaction of the Zn target shows that increasing the ion energy from 500 eV to 1000 eV increases the sputter yield from 6.47 atoms/ion to 10.38 atoms/ion. The sputter yield from the modeled sample surface, however, only increases from 5.26 atoms/ion to 7.46 atoms/ion. This lesser increase should indicate that at higher energies there is an overall increased deposition, leading to the shown effects of energy on surface morphology. XPS results, however, show that there is no clear relationship between Zn concentration and the energy used.

Fluence Effects on Surface Morphology

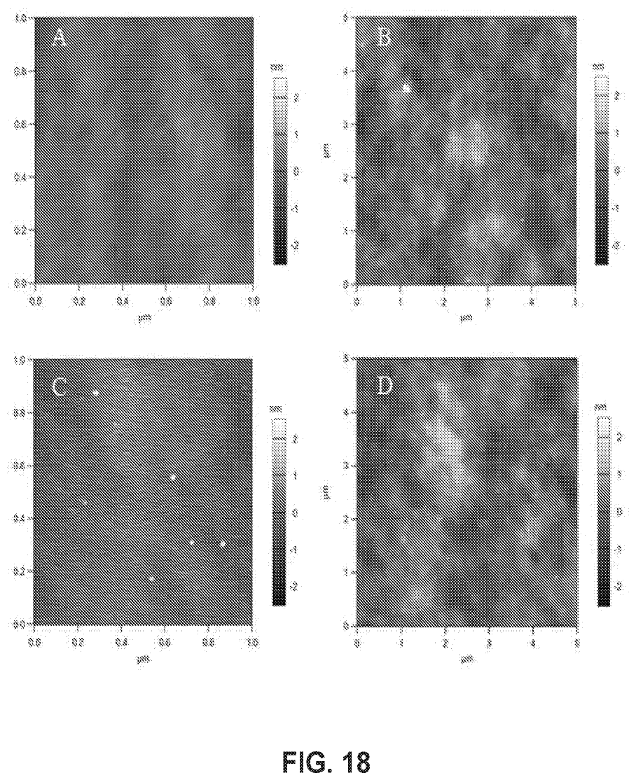

The final experimental parameter that is varied for codeposition of Zn on Si is the total fluence. All previous experiments are defined by an end irradiation fluence of 1E17 ions/cm², with the depositing beam fluence varied by the flux ratio. Literature has shown that irradiated surface evolve over time, and thus the results presented so far only indicate a single point during the evolution of the surface morphology. To examine this effect, two experiments were performed wherein codeposition was performed on Si with 500 eV and 1000 eV O₂⁺ irradiation with a flux ratio of 1.0 and a final fluence of 5E17 ions/cm². FIG. 18 compares the surfaces of both fluences at 500 eV.

First, a higher density of dots is shown to form at the higher fluence, suggesting that the surface of the sample that was stopped at 1E17 ions/cm² had not reached an equilibrium state at the time the experiment ended. More significantly, however, is the evolution of the entire surface around the dots. Ripples with a very small wavelength are clearly distinguishable. Scans were repeated with a 90° rotation to confirm that they are not an artifact of the AFM tip.

FIG. 18 shows AFM scans of Zn codeposition on Si, deposition with 500 eV O₂⁺, sample irradiated with 500 eV O₂⁺ to 1E17 ions/cm² (top left, right) and 5E17 ions/cm² (bottom left, right) with a flux ratio of 1.0.

Next, the same comparison of 1E17 ions/cm² and 5E17 ions/cm² is made for 1000 eV O₂⁺ with a flux ratio of 1.0. Here, there is not a significant difference in the size or density of dots formed over the surface, as seen in FIG. 4.12. Ripple formation is again observed when the sample is irradiated to a fluence of 5E17 ions/cm² that was not seen before. These were also confirmed by repeating the scan with a 90° rotation. The wavelength of the ripples is slightly larger in this case, which is a direct effect of the ion energy or an indirect effect of higher Zn deposition.

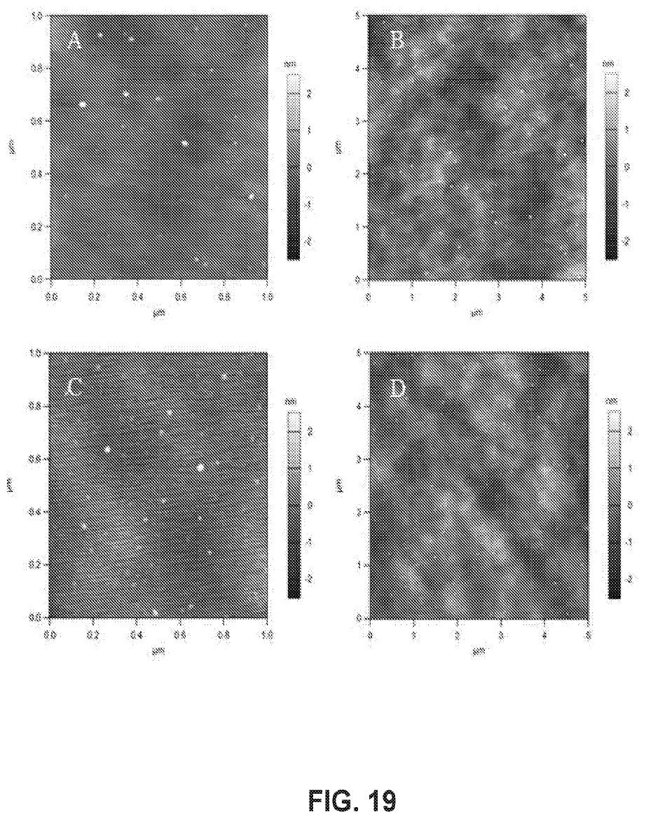

FIG. 19 shows AFM scans of Zn codeposition on Si, deposition with 1000 eV O₂⁺, sample irradiated with 1000 eV O₂⁺ to 1E17 ions/cm² (top, left, right) and 5E17 ions/cm² (bottom, left, right) with a flux ratio of 1.0

These results are significant as they agree with other published work on the irradiation of Si. Normal incidence irradiation of Si has been shown to not result in any pattern formation unless a threshold amount of impurity is present, leading to instability during irradiation. It is noteworthy that neither 500 eV nor 1000 eV cases form ripples at 1E17 ions/cm², yet both do at 5E17 ions/cm². This is indicative that both experimental parameter introduce enough Zn to lead to pattern formation, which is not surprising since work has shown that this threshold is typically very small, typically <5% [74]. Ripples are thus only seen at the higher fluence because the surface has not had time, after the critical amount of impurity was introduced, to fully evolve.

The effects of total fluence and energy on surface roughness are shown in FIG. 4.13. While the increase in roughness between 500 eV and 1000 eV for 1E17 ions/cm2 fluence can be attributed to the drastic difference in dot formation, the roughness for both energies at 5E17 ions/cm2 fluence is a combination of the dot and ripple patterns.

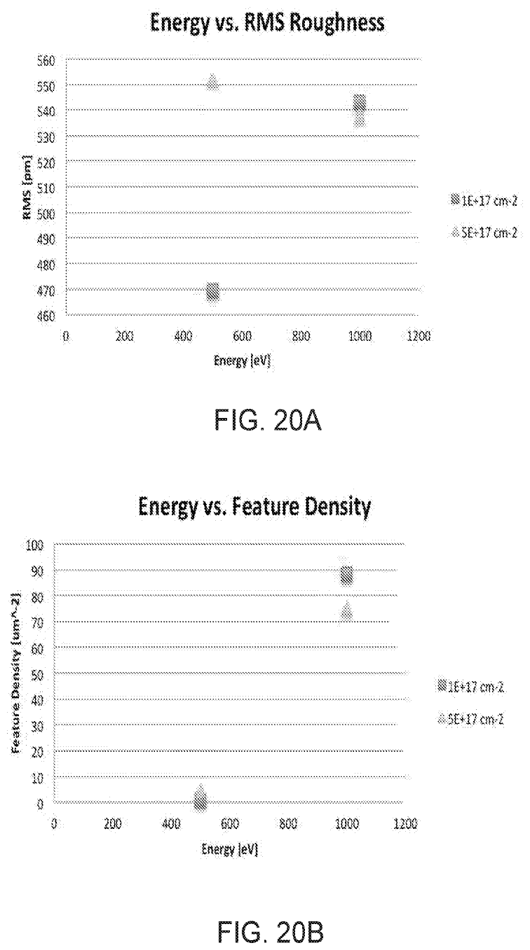

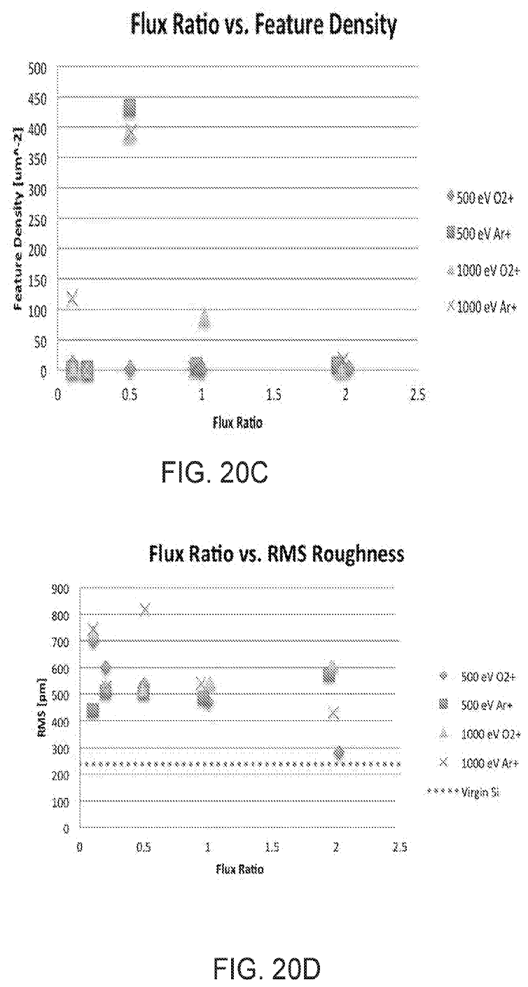

The effects on feature density, however, are much more clear. FIG. 20A shows that for both 1E17 and 5E17 ion/cm² fluence, the ion energy variation results in not only very similar changes, but also nearly identical values of density. This shows that ion energy is a significant factor in controlling density of the dot pattern, while fluence has little effect

Conclusions

This work has shown that codeposition of Zn on Si is a viable method of creating nanoscale dots over a relatively large area. All three studies have shown that, in this setup, ion beam flux ratio, energy, and fluence are significant factors in controlling surface morphology. While this control is limited to dot formation, a variety of dot sizes and densities have been demonstrated. This is further elucidated in below with chemical analysis. Less well defined, but still significant, is the effect of flux ratio on surface roughness. This data is compiled in FIG. 20D. Though not true in every case, samples are generally shown to be rougher at low flux ratios and smoother at higher ratios, which have lower deposition rates.

Ion energy is shown to have a strong effect on feature size and density, with 1000 eV creating larger, more densely packed dot patterns than 500 eV. This could be the result of a number of effects including higher energy deposition, higher sputter ratios, higher total Zn deposition, greater penetration depth, and different energy distributions throughout the surface. Total fluence was shown to not greatly affect the dot patterns, which seem to have reached equilibrium by 1E17 ion/cm² fluence, but is effective at inducing a second pattern of ripples across the surface.

The flux ratio is shown have a particularly significant effect on surface morphology, with both too high and too low of ratios resulting in little to no dot formation. In the case of Zn and Si, a ratio of 0.5 seems to be the most effective at creating surfaces with a high density of well-defined dots. This can be seen in FIG. 20C, where all but one data set has a sharp increase in feature density at the 0.5 flux ratio.

Codeposition Results on PDMS

The results of several experimental parameter sets for the codeposition of Zn on PDMS are presented and discussed herein. These match the work presented on Si to make a direct comparison between the substrates. First, the effects of ion irradiation of PDMS without deposition are examined as no published data showing the effects on surface morphology could be found. This is necessary knowledge to understand how adding deposition alters surface evolution. The first parameter space in the codeposition and irradiation work follows and focuses on the ratio between the beam fluxes/fluences. As before, this will analyze the relative ratio of deposition vs. sputter removal from the substrate. The effects of ion energy are then highlighted. Following that, results are presented for samples that were irradiated to a significantly greater fluence (5 times) to examine how the surface develops at different stages. The chapter concludes with a summary of the result of each study.

Ion Beam Patterning of Virgin PDMS

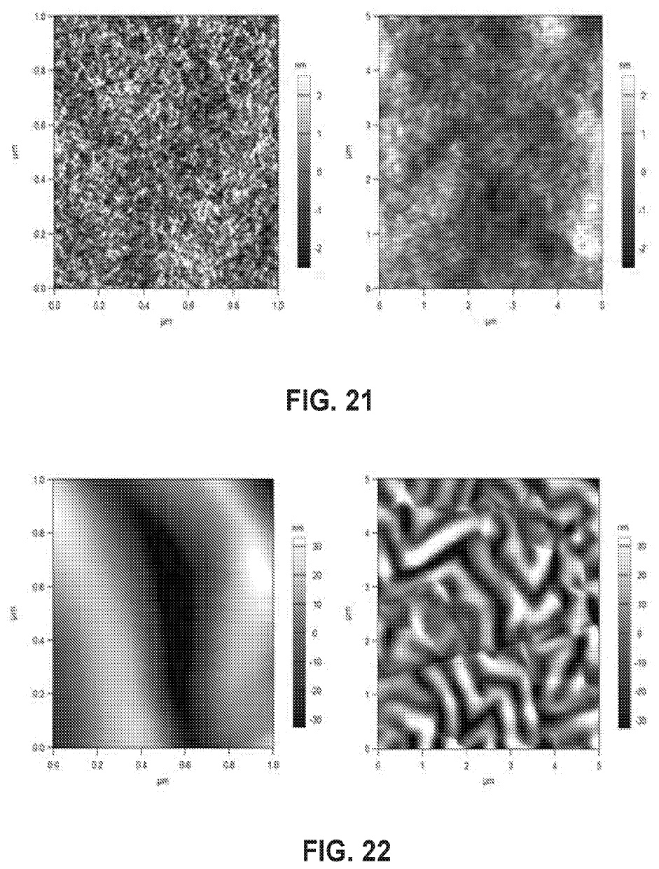

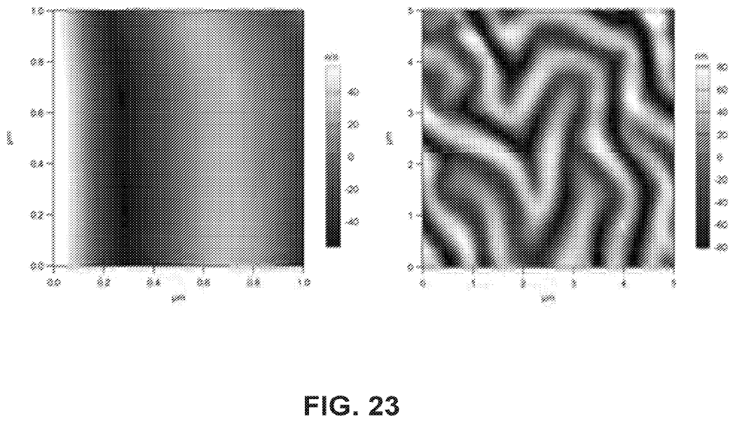

PDMS samples were first irradiated with 500 eV and 1000 eV Ar⁺ ions to a fluence of 1E17 ions/cm² to establish the effects of irradiation on surface morphology. FIG. 21 shows AFM images of a virgin PDMS samples. This shows an apparently smooth surface, though rougher than Si, with RMS roughness of 881 μm. This is undoubtedly a result of the curing process. Unlike Si, PDMS undergoes a drastic change in surface morphology after being irradiated with a normal incidence ion beam. FIGS. 22 and 23 show the changes to the surface after being irradiated to 1E17 ions/cm2 with Ar⁺ at 500 eV and 1000 eV, respectively.

Both samples show the formation of a wrinkle pattern on a much larger scale than any features created on Si. The features shown in FIG. 22 above have wrinkle peak-to-peak distances (referred to as wavelength from here on) of ˜550 nm, while those in FIG. 23 below are ˜800 nm. The 500 eV sample also shows sharp, straight borders between areas of continuous pattern formation, while the 1000 eV sample shows one continuous area.

Other than Si, the PDMS chain backbone also contains oxygen atoms, with methyl groups attached to the Si atoms. Incident ions do not only break long polymer chains into smaller components, but also create a great number of dangling bonds, creating a highly reactive surface. Additionally, the surface is originally in a state of tension from the curing process [106]. Breaking bonds allows the surface to relax, release tension, and reform chains from dangling bonds. This is believed to be the primary mechanism responsible for this surface morphology.

Ion Beam Flux Ratio

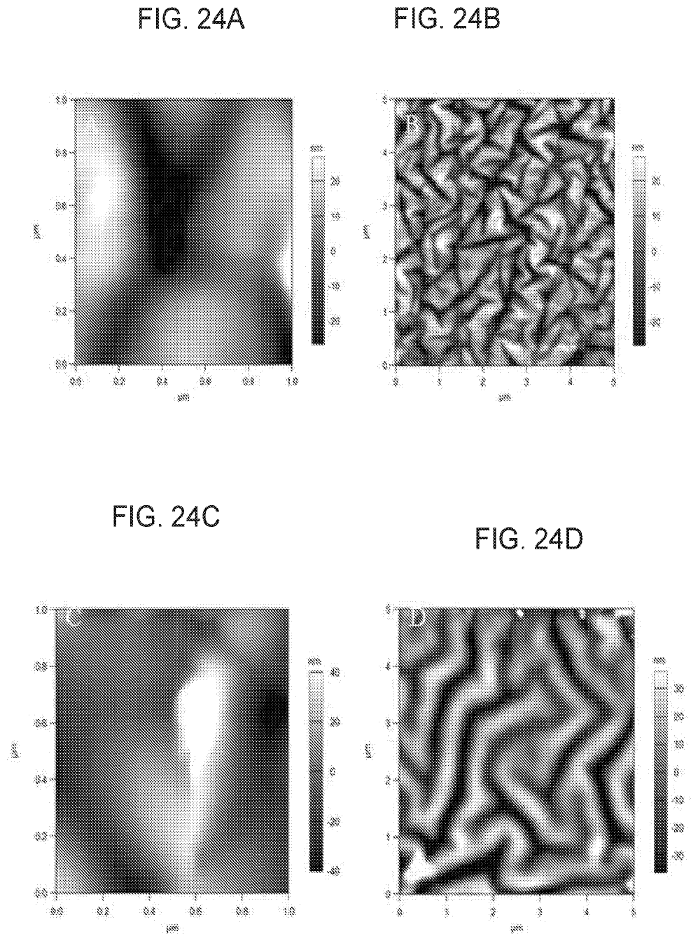

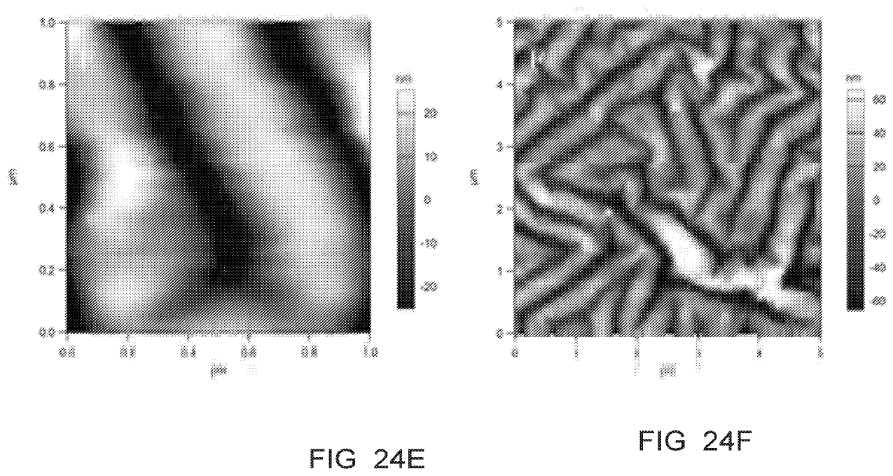

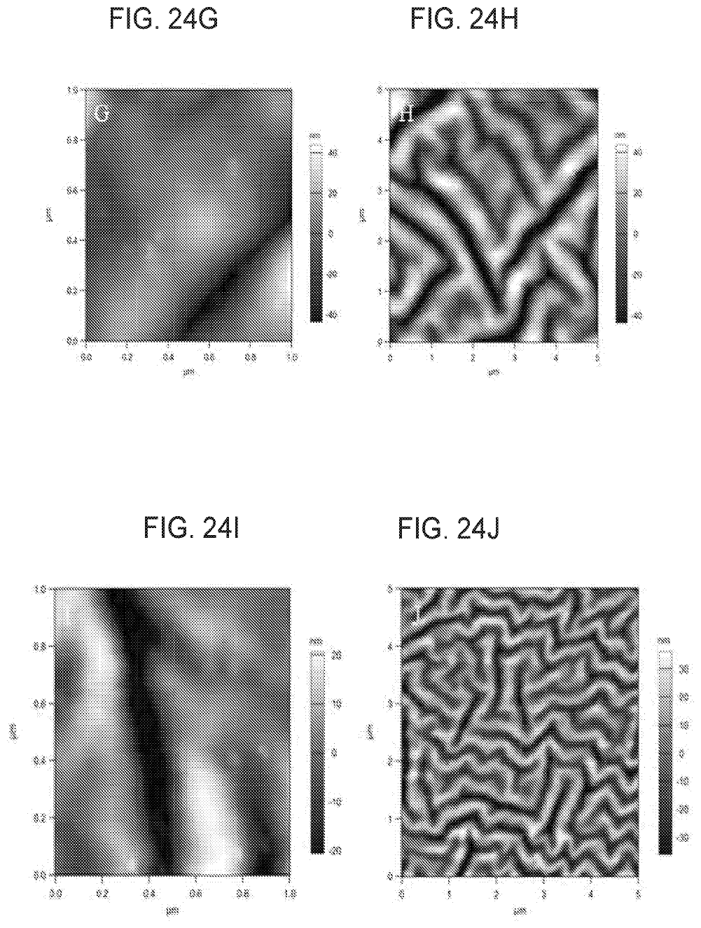

Adding Zn deposition via the codeposition setup is the logical next step following the establishment of PDMS surface response to irradiation. The ratio of ion beam fluxes is studied at 500 eV and 1000 eV ion beam irradiation with both Ar⁺ and O₂⁺ (Zn only sputter deposited with Ar⁺). Again, samples are irradiated to a fluence of 1E17 ions/cm² with deposition fluence varying with the flux ratio. The first sample set, 500 eV Ar⁺, is shown in FIGS. 24A-J and 25E.

This data confirms the initial assumption of the complexities of codepositing on a polymer. Similar to the high fluence Si experiments, two patterns have formed on the PDMS surfaces. First, there is large-scale wrinkle formation similar to the results of irradiation without deposition. On top of this, some samples show dot formation similar to that on Si. This data, however, shows a variety of permutations to the PDMS wrinkle structure. Since the only factor that varied between these samples is the deposition beam flux, the Zn must play some role in altering the wrinkle formation. Since SRIM is unable to accurately model PDMS due to complex chemistry, the actual sputter yield values are not estimated and cannot be referred to, but it is assumed that within the range of energies used in this work they will follow the same pattern of higher deposition and sputtering rates at 1000 eV compared to 500 eV.

Images A-B in FIG. 24, and image A in 25, show the lowest flux ratio, and thus highest Zn deposition. While the wrinkles still form, they are smaller than the rest of the flux ratios, with broader peaks and sharper valleys. It is theorized that the relatively high level of Zn creates a protective barrier here, limiting the penetration depth of ions into the polymer. Small, very low profile dots can be seen on these wrinkles in image 24B. Increasing flux ratio shown in C-H of FIG. 24, and B-D of 25, show wrinkle patterns similar to the no-deposition results, with only the 1.0 ratio samples showing dot formation. The highest flux ratio of 2.0 shows a higher dot density on shorter wavelength ripples.

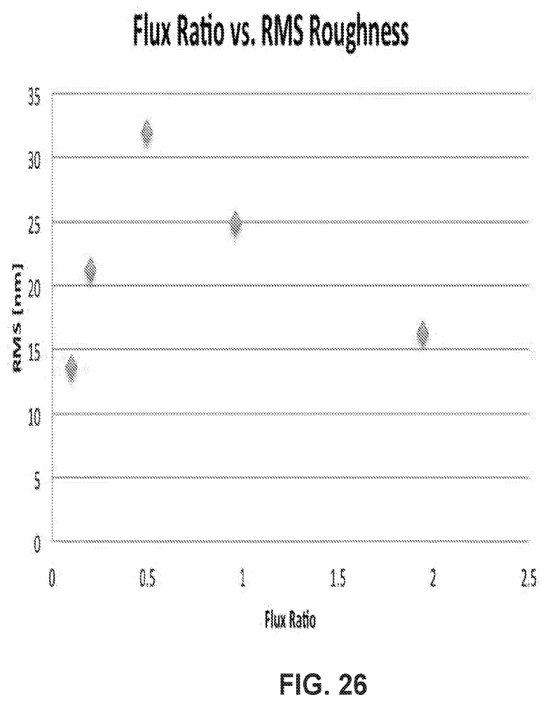

The creation of the ripple structure results in RMS roughness values orders of magnitude greater than seen on Si or the virgin PDMS surfaces, from 100s of μm to 10s of nm. This is shown in FIG. 26. Roughness data shows a sharp increase with flux ratio, reaching a peak at 0.5, then gradually reducing.

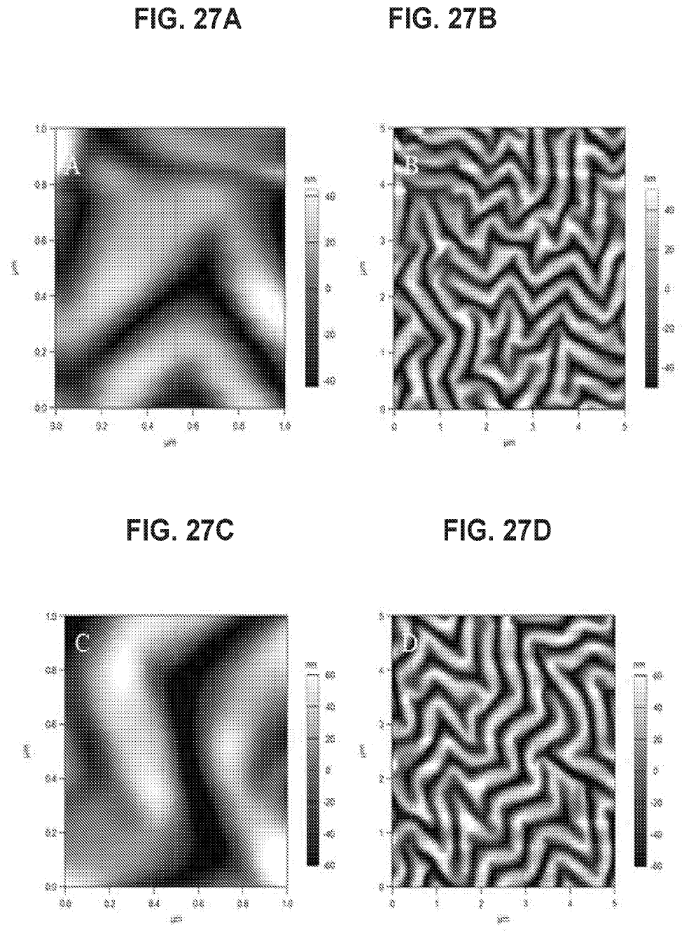

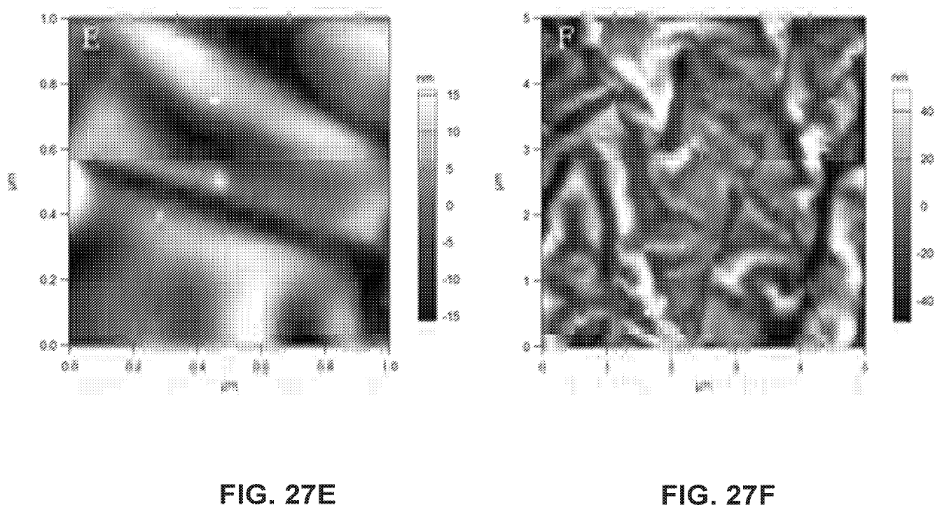

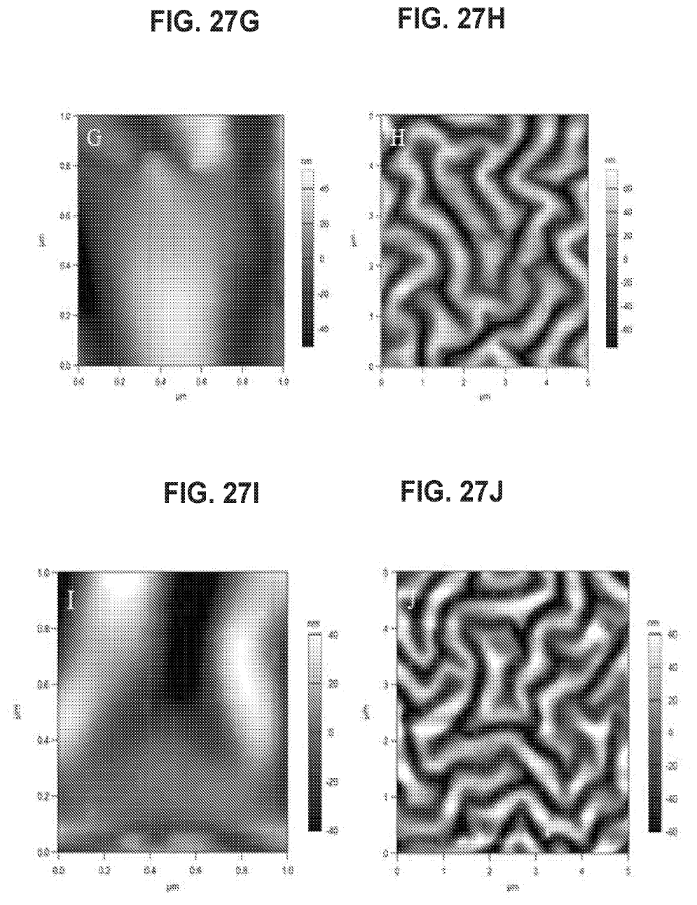

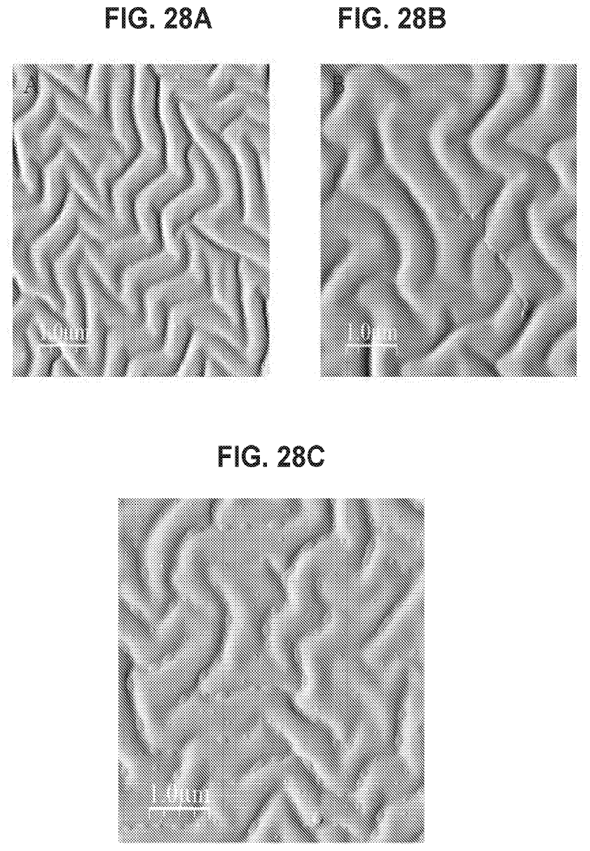

The next data set increases the Ar⁺ ion energy to 1000 eV, while maintaining the same fluence and gas species. Image A-D in FIG. 27 show the results of the lowest flux ratios, 0.1 and 0.2. These show the formation of tight wrinkle patterns with no apparent dot formation. Image 27A, however, shows that small dots have formed on the 0.2 ratio sample, with larger dots shown to be present in the ripple valleys. A flux ratio of 0.5, images 27E-F, show vague wrinkle formation with the same sharp divisions seen in FIG. 22. Atop this structure area few well defined dots. Increasing the flux ratio further shows the standard wrinkle structure, with dots forming only on the 2.0 ratio sample. Again, smaller dots can be seen on the wrinkle peaks while much larger features can be seen rising from the valleys between them, clearly shown in image C of FIG. 28.

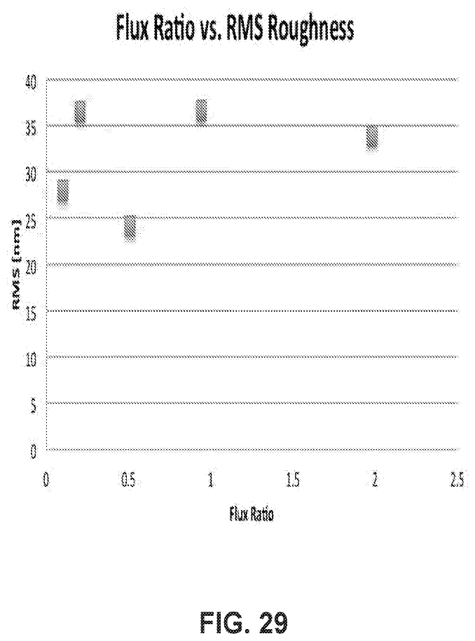

Roughness data for this sample set is shown in FIG. 29. This seems to follow the same pattern as the 500 eV set, with one notable exception in the 0.5 flux ratio sample. The rest show increased roughness with increasing ratios less than 0.5 and slowly diminishing roughness at ratios higher than 0.5. The outlier in this set corresponds to images E-F in FIG. 27, which did not form wrinkle patterns like the others. The larger plateau regions separated by short, sharp valleys resulted in this roughness data.

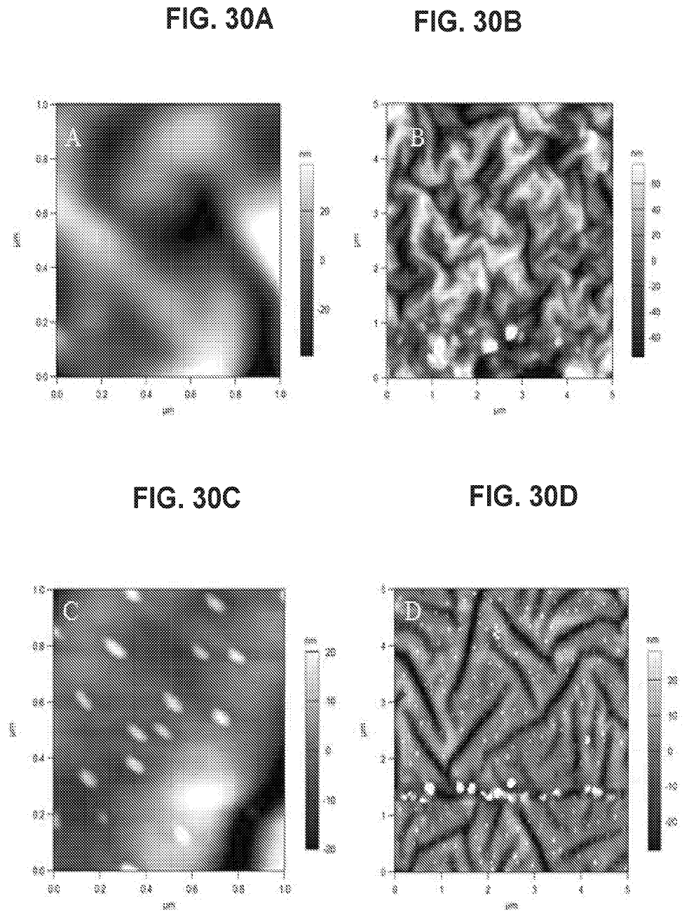

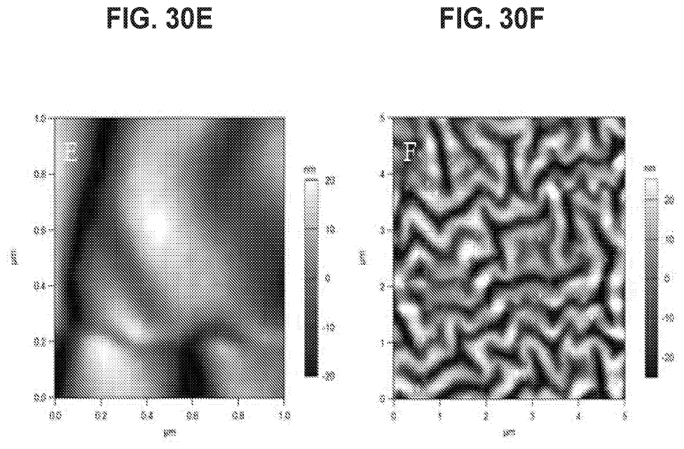

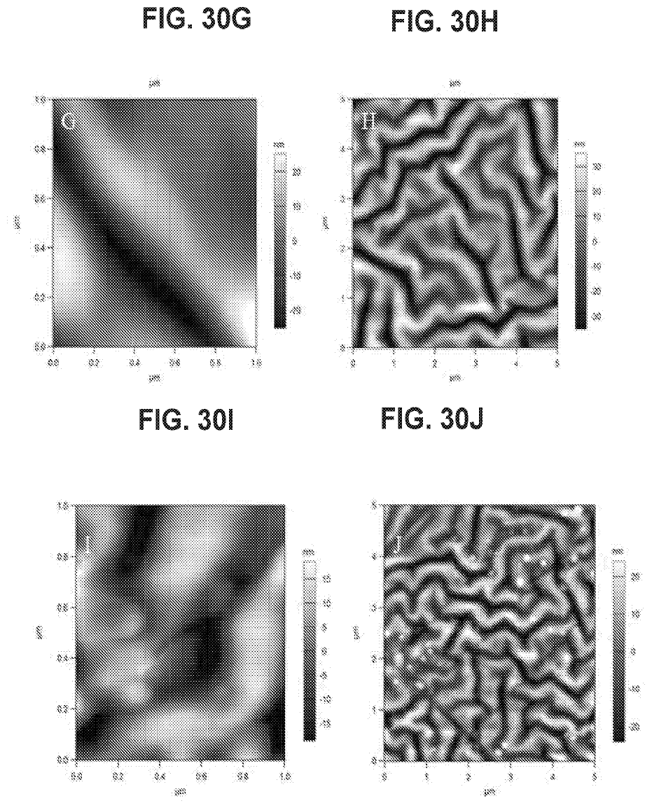

The next data set moves to using O₂⁺ ions at 500 eV and produces some exceptional results. The lowest flux ratios are shown in images A-B of FIG. 30 and show rougher wrinkles than have produced thus far with no dot formation visible in either image. The lower section of image B shows a particularly rough portion of the surface. Flux ratios of 0.2, 0.5, and 1.0 show an interesting pattern in surface evolution. All three show similar wrinkle formation, with the 0.2 flux ratio sample having large plateau regions. Dot formation is of particular significance here, with large, clearly defined dots covering the sample surface. Additionally, a sharp division cuts through the sample horizontally and contains a few large features. The 0.5 flux ratio sample, images E-F, shows dots of the same size, but much lower density. Image E appears to show a very high density of poorly defined features that may be early-stage precursors to the larger dots. The 1.0 flux ratio sample shown in images G-H, however, shows no dot formation. The highest flux ratio sample shows areas of similar wrinkle formation with some sharp dividing lines. Unlike previous samples, the larger wrinkles can be seen to cross these dividing lines, suggesting that the wrinkles were already formed when the divisions occurred. A few clusters of much larger dots (than the lower flux ratios) are also seen. These are ˜170 nm in diameter compared to ˜75 nm seen in the 0.2 and 0.5 flux ratio samples.

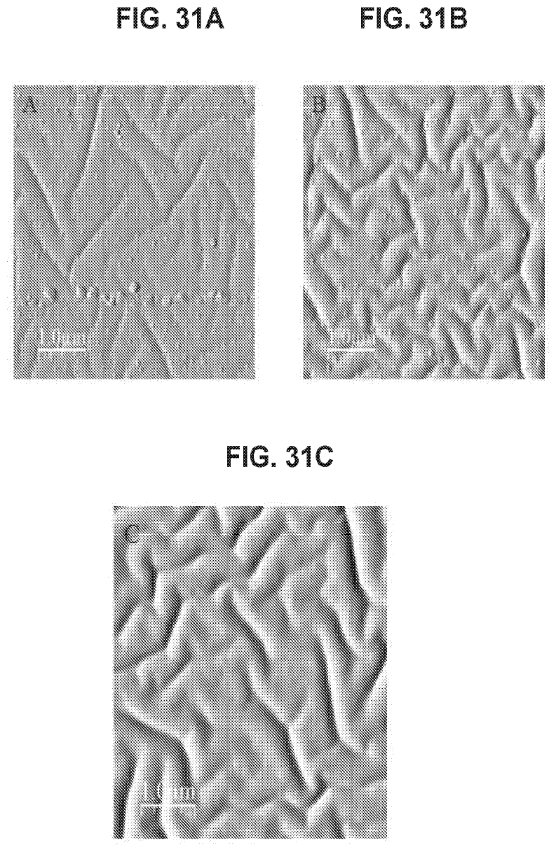

The trend in dot formation seen at flux ratios of 0.2, 0.5, and 1.0 shown in FIG. 30 are more clearly represented by the amplitude data shown below in FIG. 31.

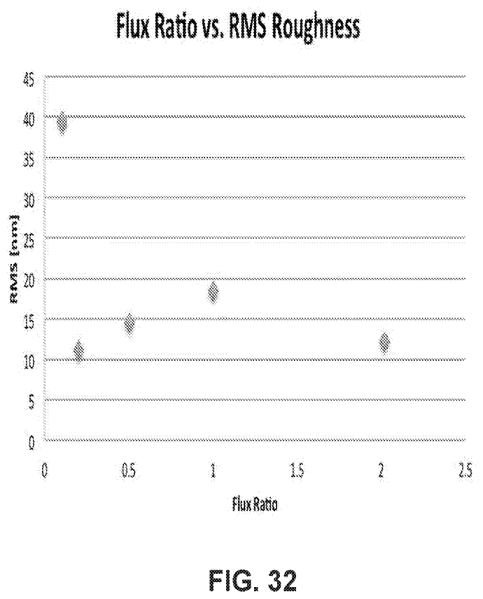

The lowest flux ratio sample shows the highest RMS roughness of this set, which can be seen by the unique rough wrinkle morphology in image B of FIG. 30. FIG. 32 shows that, after that sample, the roughness slowly increases with flux ratio, and then diminishes between the 1.0 and 2.0 ratio samples.

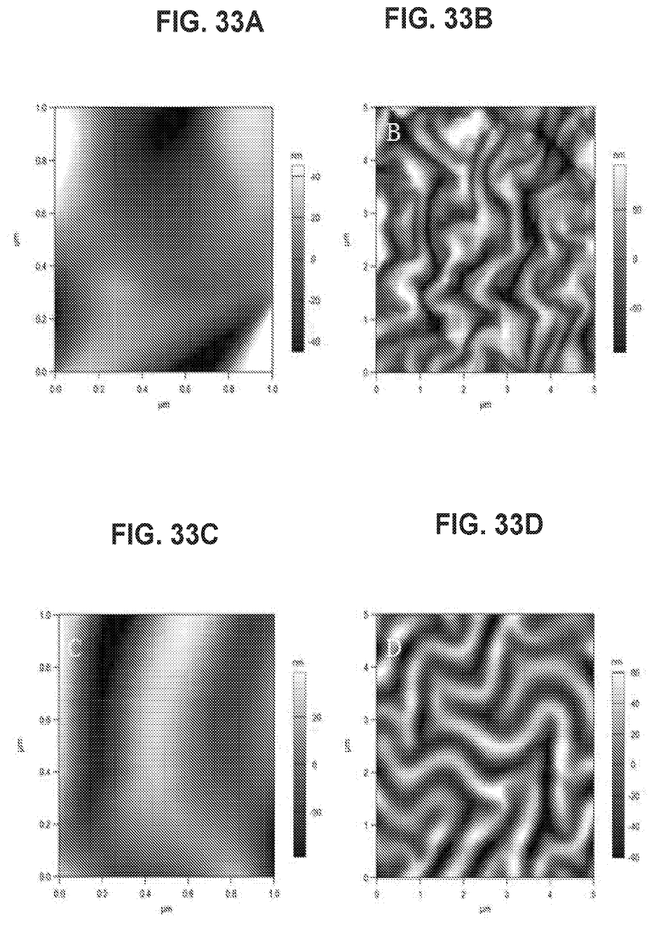

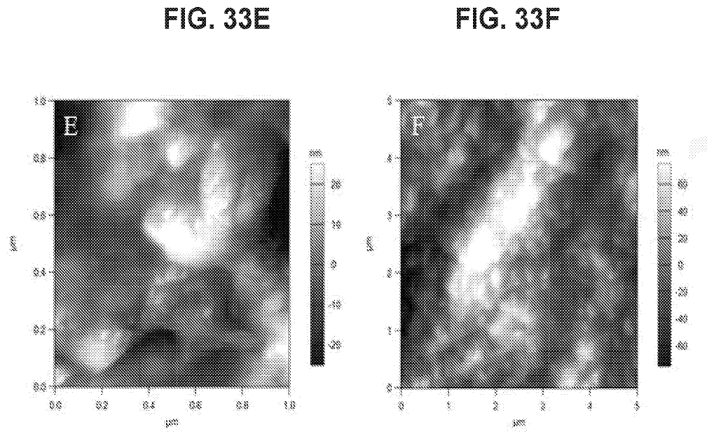

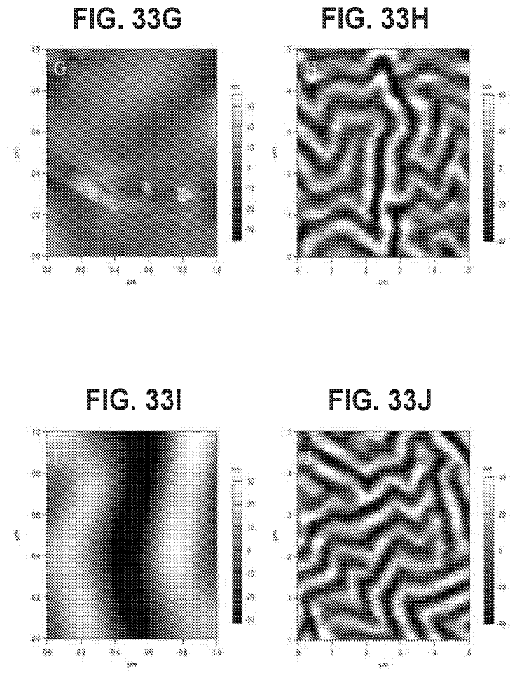

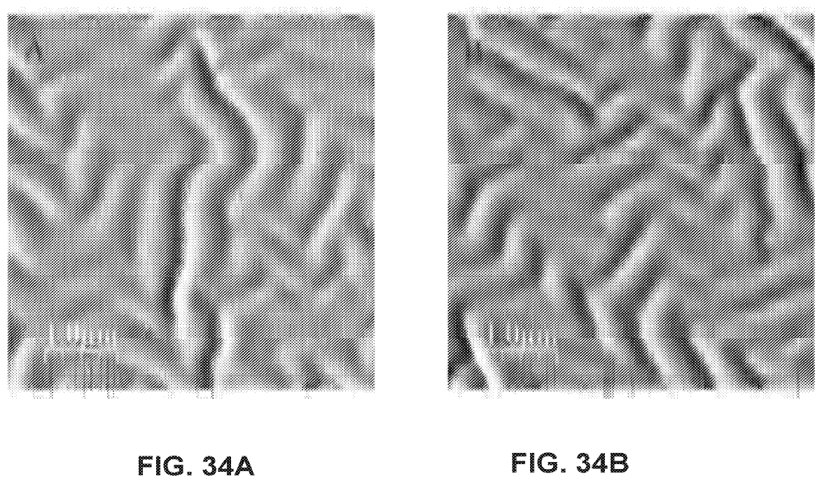

The energy of the O₂⁺ ions is then increased to 1000 eV for the next data set. Similar to the last set, the lowest flux ratio (0.1) sample, shown in images A-B of FIG. 33, formed a very rough wrinkle pattern with no dot formation. The 0.5 flux ratio sample, images E-F, also shows a very rough surface, this time with no observable wrinkles. The samples with flux ratios of 0.2, 1.0, and 2.0 all show smooth wrinkle formation with no dots on the height images. FIG. 34, however, shows that the 1.0 ratio sample does form low profile dots. The 2.0 ratio sample is also included for comparison.

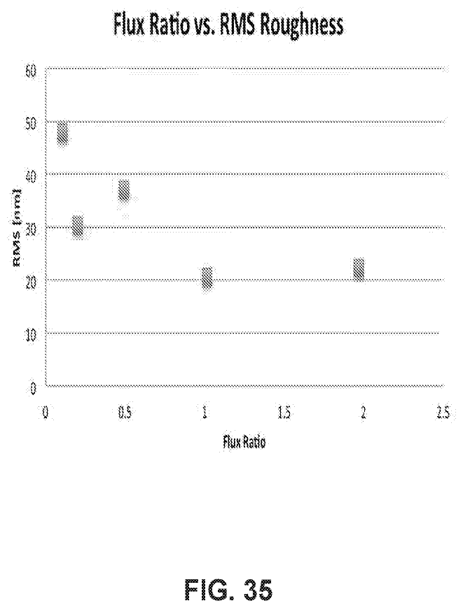

Due to the rough large-scale formation on some of the samples in this set, the RMS roughness data does not show a clear a pattern. A generally decreasing trend in roughness with increasing flux ratio can be seen in FIG. 35.

Among these data sets there is little corroborating evidence that the flux ratio plays as significant a role in surface morphology evolution as it does on Si. At the lowest flux ratio little to no dot formation is observed, with only the 1000 eV Ar⁺ sample showing well-defined wrinkle formation. Most of the rest of the data shows dots forming under individual circumstances without a clear link to the samples with adjacently higher or lower flux ratios. The 500 eV O₂⁺ set shown in FIG. 30, however, is one instance that correlates dot formation with ion beam flux ratio. Among the 0.2, 0.5, and 1.0 flux ratio samples in this set dots are clearly formed with high density at a ratio of 0.2. These diminish significantly in density in the 0.5 ratio sample and disappear completely in the 1.0 ratio sample. With the exception of a few outliers, the surface roughness among these data sets follows a roughly increasing trend with increasing flux ratio, reaching a maximum near a ratio of 1.0, then decreases.

Energy Effects on Surface Morphology

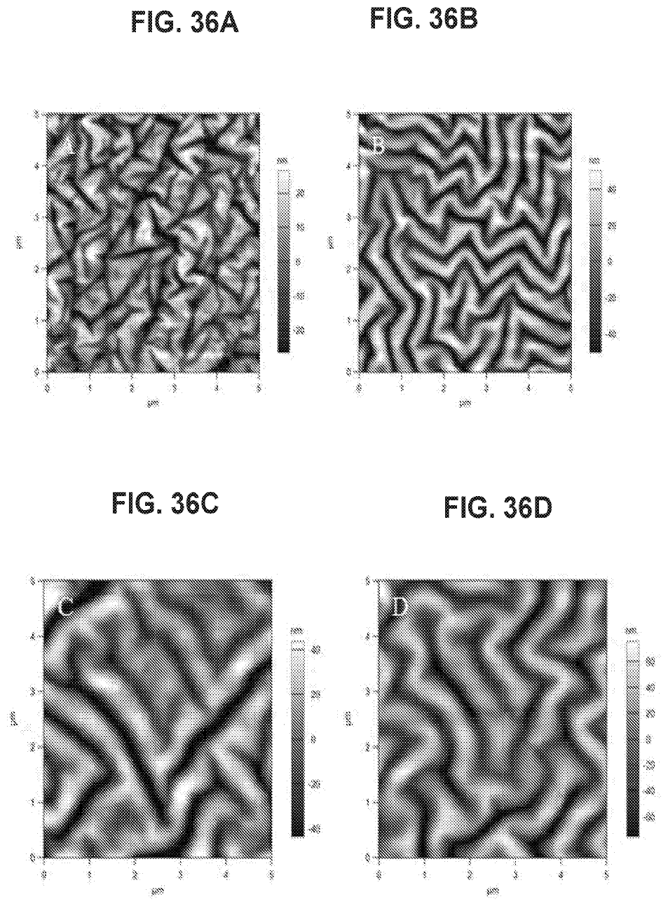

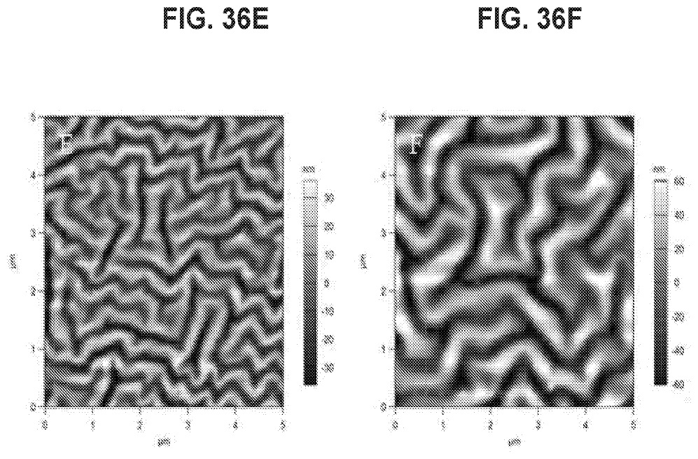

The next parameter space that is investigated is the energy of incident ions, comparing 500 eV and 1000 eV irradiations. The increase in energy results in higher Zn deposition, increased energy deposition, deeper ion penetration depth, and a different energy distribution. In the case of ion energy for codeposition on PDMS, more differences than similarities are observed. FIG. 36 shows these comparisons between 500 eV and 1000 eV Ar⁺ irradiations.

The most significant comparison can be made with the 0.1, 1.0, and 2.0 flux ratio samples from these data sets. Images 36A and 36B compare 500 eV and 1000 eV ions, respectively, with a flux ratio of 0.1. These show a rougher wrinkle patter at 500 eV and a smoother one at 1000 eV. Also, the lower energy case shows some small, faint dot while none are observed at the higher energy. Images 36C and 36D show that a flux ratio of 1.0 produces essentially the same wrinkle pattern, with low density dot formation occurring at 500 eV and none at 1000 eV. Finally, with flux ratio of 2.0, images 36E and 36F both show wrinkle and dot formation. The 500 eV sample, however, shows both smaller dots and wrinkles than the 1000 eV sample, with the dots also forming with a higher density at the lower energy. This data suggests it is more likely for dots to form under 500 eV than 1000 eV codeposition.

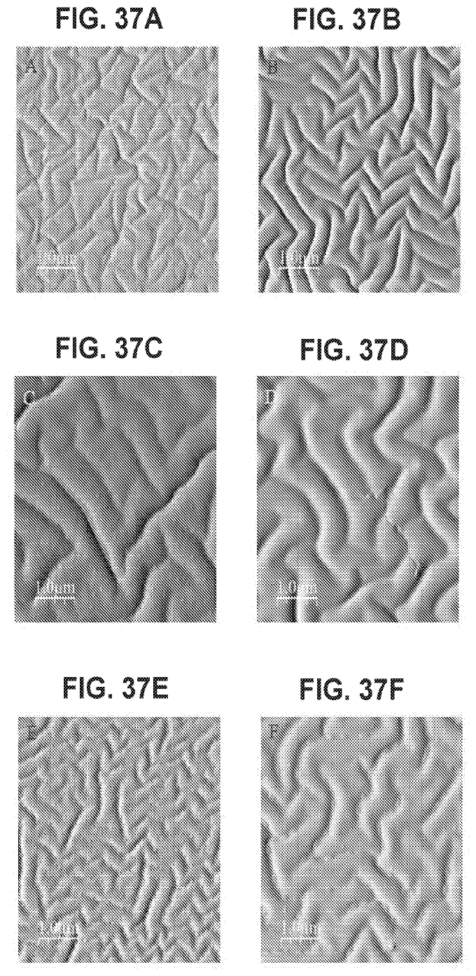

The amplitude images for this data are shown in FIG. 37. Images 37C, 37E, and 37F in particular show a clearer picture of dot formation, while the other show extremely smooth surfaces.

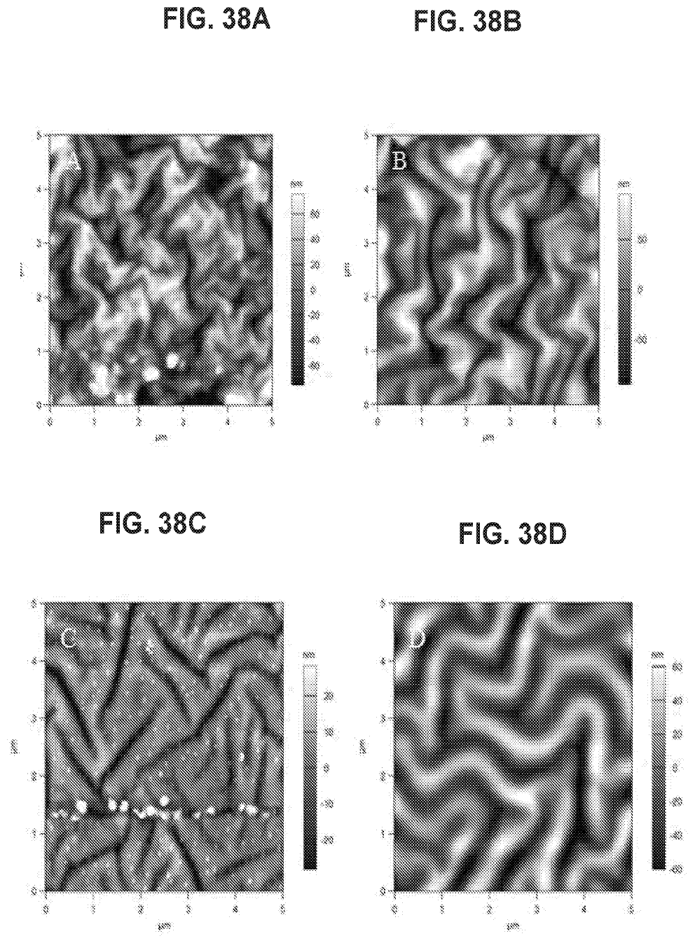

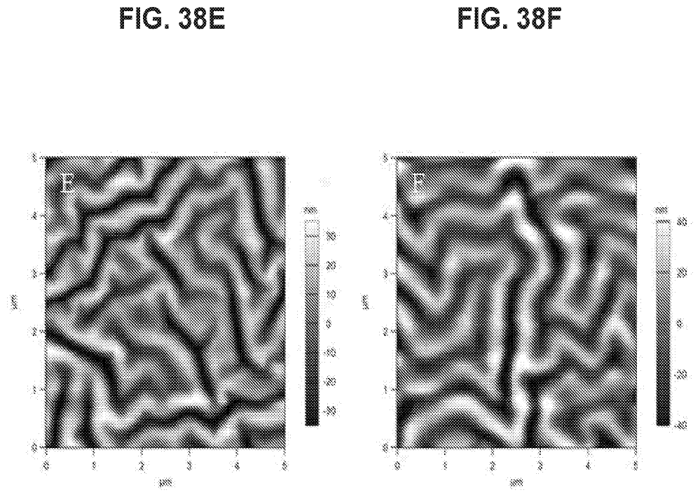

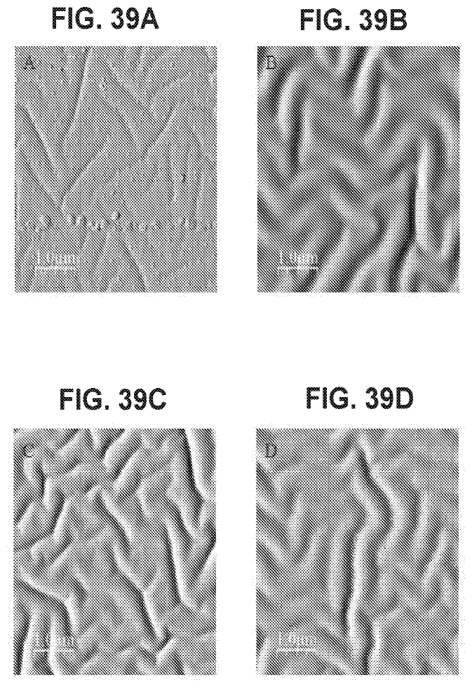

The same energy comparison is made among those samples exposed to O₂⁺ ions in FIG. 38. Here those samples with flux ratios of 0.1, 0.2, and 1.0 are highlighted. Both samples at the lowest flux ratio show notably rough wrinkle patterns, with that at 500 eV (38A) being slightly more tightly folded than the 1000 eV sample (38B). The samples with a flux ratio of 0.2 are significantly different. At 500 eV (38C), the surface forms wrinkles with large plateaus instead of rounded curves (38D). Additionally, the lower energy induces high-density dot formation while none are seen at 1000 eV. At a flux ratio of 1.0, images 38E and 38F show that there is little difference in the height data. The amplitude images of these, images 39C and 39D of FIG. 39, show that there is, in fact a difference. While the 500 eV sample shows smooth wrinkles, at 1000 eV a high density of very small dots is seen to form.

FIG. 39 shows the height data corresponding to FIG. 38. Overall, energy appears to have no significant, or at least direct, effect on surface morphology. At some flux ratios, dots are seen to form at 500 eV and not 1000, and at other ratios the opposite is seen.

Fluence Effects on Surface Morphology

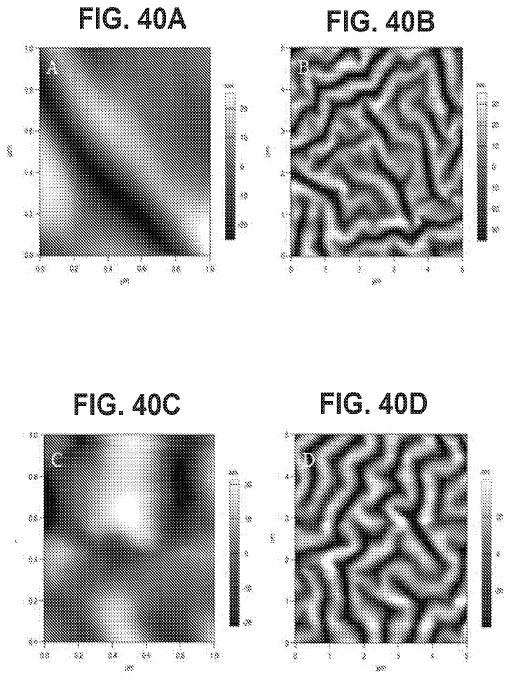

The final parameter space explored in the Zn codeposition experiments on PDMS is the overall fluence. All samples to this point were defined by a total irradiation beam fluence of 1E17 ions/cm², with the deposition beam fluence varying by the flux ratio. To determine whether further surface evolution would occur, samples were irradiated with 500 eV and 1000 eV O₂⁺ and a flux ratio of 1.0 to a final fluence of 5E17 ions/cm2. The 500 eV results are shown in FIG. 40.

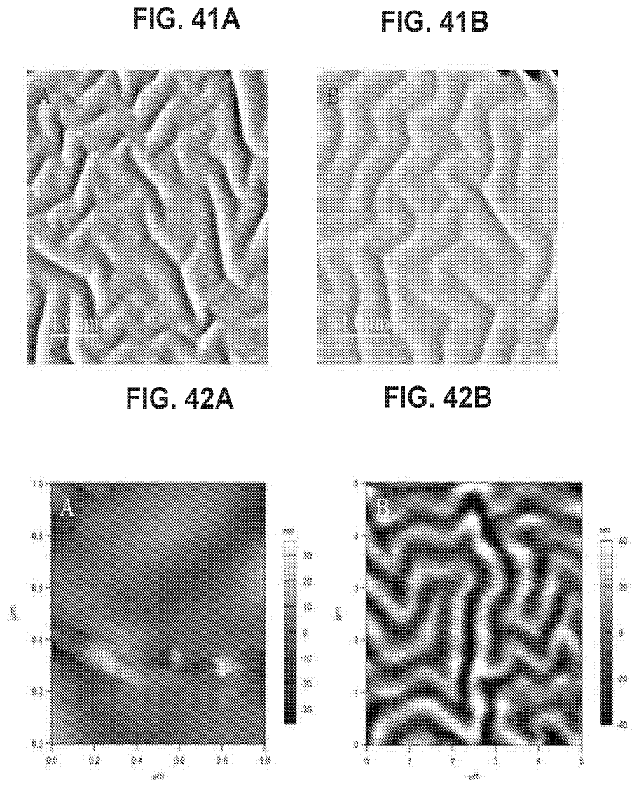

Images B and D of FIG. 40 indicate that the increased fluence had little effect on surface morphology. Both samples show well-defined wrinkle patterns, with image 40B showing that the 1E17 ions/cm² sample had some valleys that are shallower than those of the 5E17 ions/cm² sample. FIG. 41 shows that some dot formation does occur in the valleys at the higher fluence. This suggests that the surface had not yet reached an equilibrium morphology by a fluence of 1E17 ions/cm². The additional fluence appears to have allowed for the coalescence of deposited material in the wrinkle valleys.

FIG. 42 shows this comparison for 1000 eV O₂⁺ irradiation with notably different results.

Images A-B of FIG. 42 show that at a fluence of 1E17 ions/cm² the surface forms clear ripples with only small dots. The dots can be seen more clearly in image A of FIG. 43. At a fluence of 5E17 ions/cm², however, features are seen. Image 42D and 42E are both from the same sample, but at different locations on the surface. Other samples were scanned at various locations, with no notable difference in morphology. Image 42D shows a very high density of small features forming on the wrinkles while 42E shows a lower density of dots with two distinct sizes. Small dots, similar to other samples, are seen to cover the surface. There are also larger dots that only appear in the wrinkle valleys. The cause of this difference is unknown, but could be due to slight differences in deposition based on distance to the sputtering target. This would imply that there is a very fine dependence of dot formation on Zn deposition. This makes identifying precise parameters difficult, while demonstrating that the ability to tightly control surface morphology is possible.

Conclusions

The codeposition of ZnO on PDMS has demonstrated that a variety of surface morphologies are achievable. In all cases, irradiation is shown to induce relatively large-scale wrinkles on the surface. The effects of codeposition are shown to produce dots in some, but not all cases. Among the studies of flux ratio, energy, and total fluence, no one parameter is shown to directly control the evolution of dots on the surface. Flux ratio is shown to have an effect in the case of 500 eV O₂⁺ at ratios of 0.2-1.0. Here, a transition was seen from clearly formed dots, to a lower density of dots, to a smooth surface that was devoid of features other than the wrinkles.

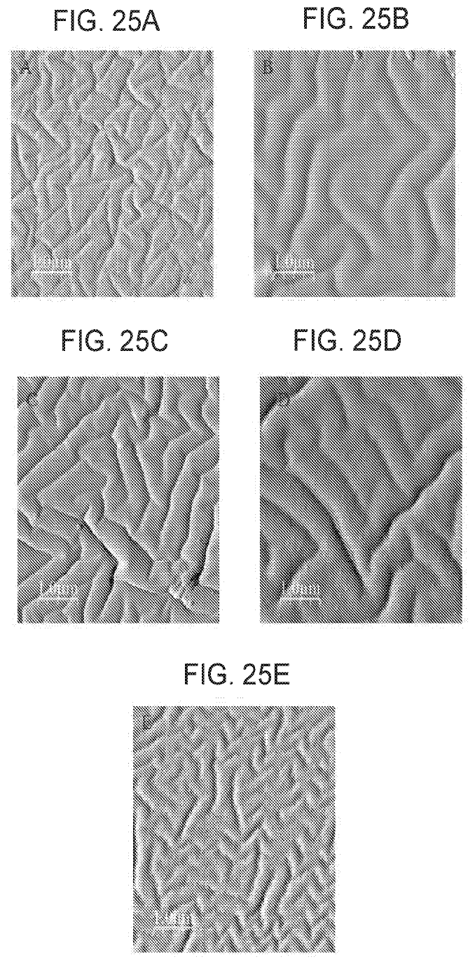

One very notable result seen in many cases of dot formation on PDMS is the preference of formation in valleys. For samples with smaller dots, they are shown to be created evenly over the surface. Examples of this can be seen in FIG. 25, image D, FIG. 311, image B, and FIG. 34, image A. For samples specifically with a low density of dots, and those with larger dots, the preference of valleys is quite clear. These can be seen in FIG. 38, images A and C, FIG. 41, image B, and FIG. 43, image C. These images are organized in FIG. 44 for direct comparison. A few samples show a high dot density over the entire sample, but also show a preference for valley formation, such as FIG. 25 image E and FIG. 41 image A. Although deposition should occur evenly over the surface, there is a preference, specifically when more material is present, for coalescence to occur in the valley. This is evidence that it is energetically preferable for the deposited, and concurrently oxidized, Zn to gather in these regions. In the valley, the curvature allows the material to form a more spherical geometry, as opposed to the peaks, where migrating material is stretched over the curve in a flatter configuration. It is also possible this occurs due to a geometric argument in terms of the irradiating ion beam. As ions collide with surface atoms, they are imparted with momentum inward toward the surface. This momentum may drive the deposited material away from the peaks and cause them to collect in the valleys.

Chemical Analysis and Wettability Results

The analysis thus far has focused on how the processing parameters have affected the surface morphology of Si and PDMS under codeposition. The effects of both Zn deposition and the irradiation of the substrates with Ar⁺ and O₂⁺ ions also resulted in the changes in surface chemistry, which are presented here via XPS. This includes the comparisons of how flux ratio affected overall deposition, comparisons of how Ar⁺ and O₂⁺ interact with the substrates, and how these relate to the morphologies shown in the figures. Additionally, contact angle results are presented.

Surface Atomic Concentrations after Codeposition on Si

For each sample, a survey XPS scan was performed that indicated the presence of significant peak, as described earlier. This is shown in FIG. 44 for Si that underwent codeposition with 500 eV O₂⁺ to an irradiation fluence of 5E17 ions/cm2 with a flux ratio of 1.0. A number of Zn, O, Si, and C peaks can be seen in this. The presence of C contamination was detected on all samples. This is attributed to analysis being performed ex-situ and a thin film of contamination attaching to the surface between removal from the experimental vacuum chamber and insertion in the XPS vacuum chamber. From the work performed here, however, it is impossible to tell if C was present during the experiment, and thus could have influenced the experimental results. If it did form after the experiment, this will not only create the C peak, but could also slightly diminish the signal from underlying surface atoms, particularly Zn, which is shown to have low concentrations. The discussion of the results will focus on the Zn and O concentrations detected on the surface, but presence of C should not be ignored.

Following the survey scan, region scans were performed on the identified peaks. These were taken more slowly, with lower pass energy, and repeated 10 times to produce smooth average peaks for analysis. FIG. 45 shows the region scans for the leftmost Zn peaks in FIG. 44. The rough line represents the measured data, while the two smooth lines are expected fittings for those peaks identified by CasaXPS. The area within the peaks was then summed and, after correcting for the sensitivity factors of each element based on the library within CasaXPS, used to calculate the atomic percent of each identified element. The complete list of Zn, O, Si, and C concentrations found on all samples is presented in Table 1A and Table 1B.

TABLE 1A
Atomic Concentration after Codeposition on Si Substrates

| Irradiation | Energy | | Fluence | | | | |
|---|---|---|---|---|---|---|---|
| Species | [eV] | Flux Ratio | [ions/cm²] | Zn % | O % | Si % | C % |
| Virgin | | | | | 30.79 | 54.89 | 14.31 |
| O₂⁺ | 1000 | 1.015 | 1E+17 | 1.83 | 20.6 | 7.89 | 69.69 |
| O₂⁺ | 500 | 1.002 | 1E+17 | 6.85 | 44.58 | 21.86 | 26.71 |
| O₂⁺ | 500 | 0.502 | 1E+17 | 2.94 | 45.53 | 28.19 | 23.34 |
| O₂⁺ | 1000 | 0.492 | 1E+17 | 7.8 | 24.49 | 1.41 | 66.29 |
| O₂⁺ | 500 | 2.020 | 1E+17 | 0.43 | 54.62 | 28.46 | 16.48 |
| O₂⁺ | 1000 | 1.968 | 1E+17 | 2.94 | 50.06 | 23.01 | 23.99 |
| Ar⁺ | 500 | 0.962 | 1E+17 | 1.5 | 42.41 | 24.44 | 31.65 |
| Ar⁺ | 1000 | 0.943 | 1E+17 | 5.47 | 28.44 | 9.42 | 56.68 |
| O₂⁺ | 500 | 0.986 | 5E+17 | 6.48 | 28.84 | 9.03 | 55.65 |
| O₂⁺ | 1000 | 0.993 | 5E+17 | 4.47 | 38.37 | 14.72 | 42.22 |
| O₂⁺ | 500 | 0.104 | 1E+17 | 5.52 | 22.39 | 0.39 | 71.71 |
| O₂⁺ | 1000 | 0.108 | 1E+17 | 17.14 | 33.48 | 5.67 | 43.73 |
| O₂⁺ | 500 | 0.2020 | 1E+17 | 13.82 | 39.85 | 12.22 | 34.11 |
| O₂⁺ | 1000 | 0.2002 | 1E+17 | 0.66 | 30.56 | 20.27 | 48.51 |
| Ar⁺ | 500 | 1.945 | 1E+17 | 4.81 | 42.59 | 21.23 | 31.37 |
| Ar⁺ | 1000 | 1.980 | 1E+17 | 2.2 | 50.52 | 25.09 | 22.19 |
| Ar⁺ | 500 | 0.202 | 1E+17 | 1.26 | 32 | 17.67 | 49.07 |
| Ar⁺ | 1000 | 0.204 | 1E+17 | 8.54 | 31.02 | 14.1 | 46.34 |
| Ar⁺ | 500 | 0.100 | 1E+17 | 5.82 | 33.15 | 19.56 | 41.48 |
| Ar⁺ | 1000 | 0.100 | 1E+17 | 10.49 | 27.59 | 10.2 | 51.72 |
| Ar⁺ | 500 | 0.497 | 1E+17 | 3.34 | 35.17 | 31.14 | 30.35 |
| Ar⁺ | 1000 | 0.508 | 1E+17 | 4.96 | 30.52 | 25.49 | 39.02 |

TABLE 1B
Atomic Concentration after Codeposition on PDMS Substrates

| Irradiation | Energy | | Fluence | | | | |
|---|---|---|---|---|---|---|---|
| Species | [eV] | Flux Ratio | [ions/cm²] | Zn % | O % | Si % | C % |
| Virgin | | | | 0 | 27.88 | 22.27 | 49.85 |
| O₂⁺ | 1000 | 1.015 | 1E+17 | 2.23 | 27.81 | 12.15 | 27.74 |
| O₂⁺ | 500 | 1.002 | 1E+17 | 1.99 | 30.82 | 14.18 | 22.19 |
| O₂⁺ | 500 | 0.502 | 1E+17 | 2.08 | 34.34 | 17.22 | 46.37 |
| O₂⁺ | 1000 | 0.492 | 1E+17 | 0.26 | 29.45 | 21.77 | 48.53 |
| O₂⁺ | 500 | 2.020 | 1E+17 | 0.17 | 43.26 | 23.23 | 33.34 |
| O₂⁺ | 1000 | 1.968 | 1E+17 | 1.74 | 44.35 | 21.69 | 32.23 |
| Ar⁺ | 500 | 0.962 | 1E+17 | 0.97 | 42.66 | 19.87 | 36.51 |
| Ar⁺ | 1000 | 0.943 | 1E+17 | 0.16 | 27.85 | 21.96 | 50.03 |
| O₂⁺ | 500 | 0.986 | 5E+17 | 1.32 | 33.01 | 18.09 | 47.58 |
| O₂⁺ | 1000 | 0.993 | 5E+17 | 0.98 | 35.72 | 20.14 | 43.16 |
| O₂⁺ | 500 | 0.104 | 1E+17 | 2.87 | 30.86 | 17.78 | 48.49 |
| O₂⁺ | 1000 | 0.108 | 1E+17 | 3.15 | 33.81 | 17.9 | 45.14 |
| O₂⁺ | 500 | 0.2020 | 1E+17 | 0.31 | 27.52 | 19.29 | 52.88 |
| O₂⁺ | 1000 | 0.2002 | 1E+17 | 0.66 | 30.56 | 20.27 | 48.51 |
| Ar⁺ | 500 | 1.945 | 1E+17 | 0.71 | 40.98 | 22.23 | 36.08 |
| Ar⁺ | 1000 | 1.980 | 1E+17 | 0.15 | 39.57 | 22.6 | 37.68 |
| Ar⁺ | 500 | 0.202 | 1E+17 | 1.26 | 32 | 17.67 | 49.07 |
| Ar⁺ | 1000 | 0.204 | 1E+17 | 0.46 | 31.97 | 20.14 | 47.42 |
| Ar⁺ | 500 | 0.100 | 1E+17 | 0.3 | 31.31 | 20.1 | 48.29 |
| Ar⁺ | 1000 | 0.100 | 1E+17 | 0.51 | 30.66 | 19.32 | 49.51 |

FIG. 46 as it relates to the flux ratio shows the atomic concentration of Zn on Si after codeposition. This data contains a number of interesting details. First, a higher concentration of Zn is shown to be deposited at the lowest flux ratio for most codeposition conditions. This then decreases as the flux ratio increases. This confirms two important points: first, that the flux ratio does in fact control the deposition of Zn and, second, that lower flux ratios result in higher Zn deposition while higher ratios result in lower Zn deposition. Recall that the ratio of fluxes is defined as the flux of the substrate irradiating ion beam divided by the flux of the sputter depositing beam. Thus, lower flux ratios correspond to a higher flux impinging on the Zn target compared to that flux impinging on the substrate surface. Though the trend of Zn concentration with flux ratio is generally true, it can be seen to not be universally true. The case of 500 eV Ar⁺ is of particular note, as it appears to show no trend at all. This could be due to experimental error. At several of the flux ratios, such as 0.1 and 0.5, the Zn concentration is higher for 1000 eV codeposition than 500 eV. At others however, 0.2, 1.0, and 2.0 this is not seen, differing from the modeling results that suggest that higher deposition rates should be achieved at 1000 eV.

The atomic concentration of O on Si is shown in FIG. 53. Here, a different pattern is seen. In all cases the highest flux ratio results in the highest O content on the surface, suggesting that a lower Zn deposition rate has an effect on the sputtering of oxygen. This could occur if the Zn binds with O, forming ZnO and a stronger bond, thus a lower sputter yield. For both Ar⁺ and O₂⁺, there is a clear increase in O concentration with increasing flux ratio, agreeing with the aforementioned argument. Neither data set at 1000 eV, however, shows this pattern, with Ar⁺ staying relatively constant up to a flux ratio of 1.0, and O₂⁺ actually decreasing. This decrease in O after O₂⁺ irradiation is particularly unexpected and could be a result of the C contamination.

In addition to flux ratio, the total fluence during codeposition was examined by irradiating samples to both 1E17 ions/cm² and 5E17 ions/cm². The compositional results for this test on Si are shown in FIG. 48. Here, after codeposition with 500 eV O₂⁺, the O concentration remains relatively constant, while a significant increase seen at 1000 eV. This suggests that a stable surface chemistry was achieved by 1E17 ions/cm² fluence at 500 eV, while the chemistry was still evolving at 1000 eV. This is interesting in terms of the surface morphology from Si. FIGS. 18 and 19 show significant differences between these two fluences. At 500 eV, the surface was still smooth at 1E17 ions/cm² and developed both dots and ripples at 5E17 ions/cm². At 1000 eV, dot patterns are shown at both fluences, but similar to the lower energy, ripples only form at the higher fluence In addition to flux ratio, the total fluence during codeposition was examined by irradiating samples to both 1E17 ions/cm² and 5E17 ions/cm². The compositional results for this test on Si are shown in FIG. 6.5. Here, after codeposition with 500 eV O₂⁺, the O concentration remains relatively constant, while a significant increase seen at 1000 eV. This suggests that a stable surface chemistry was achieved by 1E17 ions/cm² fluence at 500 eV, while the chemistry was still evolving at 1000 eV. This is interesting in terms of the surface morphology from Chapter 4. FIGS. 18 and 19 show significant differences between these two fluences. At 500 eV, the surface was still smooth at 1E17 ions/cm² and developed both dots and ripples at 5E17 ions/cm². At 1000 eV, dot patterns are shown at both fluences, but similar to the lower energy, ripples only form at the higher fluence.

The O concentration for these samples is presented in FIG. 49. At 500 eV, where Zn concentration was stable, the O concentration actually decreases significantly. At 1000 eV the opposite is seen as both the Zn and O concentrations increase.

As with Si, a survey XPS scan was first performed on the PDMS samples to identify relevant peaks. This is shown in FIG. 50 for both a virgin PDMS surface and one that has undergone codeposition with 1000 eV O₂⁺ to 1E17 ions/cm² with a flux ratio of 0.1.

After the relevant peaks were identified, they were then scanned more slowly with region scans multiple times to produce smooth peaks for compositional analysis. Two examples of this are shown in FIGS. 51A and 51B. Image 51A shows the same two Zn peaks highlighted in FIG. 45 on Si. Image 51B shows a compounded peak from two Si photoelectrons that are close in energy.

The compositional analysis of the PDMS sample notably shows a surface stoichiometry that very nearly matches the PDMS monomer. Each monomer contains 1 Si atom, 1 O atom, 2 C atoms, and 6 H atoms. The XPS atomic concentration for the virgin sample showed 22.27% Si, 27.88% Si, and 49.85% C. Hydrogen is not directly detectable by XPS. It must be considered, however, that it is just as likely that contamination formed on PDMS samples between experiment and analysis. This result then may not be entirely reliable in showing the predicted stoichiometry of virgin PDMS.

FIG. 52 shows the Zn concentration on PDMS after codeposition. As before, there is a general trend of decreasing Zn with increasing flux ratio, as expected. Except for the 0.2 flux ratio samples, 500 and 1000 eV O₂⁺ and 1000 eV Ar⁺ all follow this trend. Once again, there is not a straightforward relationship between energy and species and the surface concentration of Zn. It is quite interesting, however, that the overall percentage of Zn found on the surface of these samples is notably lower than on Si, where the concentration reached 17.14%. This would suggest that Zn/ZnO does not bind readily to the PDMS surface and is more easily sputtered. This is unexpected as the PDMS surface should be more chemically active with many broken polymer chains.

The O concentration for these samples is shown in FIG. 52. Again, there is a generally increasing trend in O concentration with increased flux ratio, with similar values as those seen on Si. It is again interesting that there is not an increased amount of O in those samples irradiated with O₂⁺ compared to those irradiated with Ar⁺.

In the case of PDMS, increasing fluence from 1E17 ions/cm² to 5 E 17 ions/cm² decreases the Zn concentration from ˜2% to ˜1%, as seen in FIG. 54. While this does demonstrate a common trend between the two energies, it also does not match what was demonstrated on Si and, more importantly, represents a very small difference that could easily be attributed to analysis error.

The same analysis for O concentration as it relates to fluence is shown in FIG. 55. Interestingly, both energies again follow a very close trend, this time increasing with fluence. Here, the increases are more significant, with a 2.20% increase at 500 eV and 7.91% increase at 1000 eV.

Surface Wettability

In addition to surface morphology and chemistry, surface wettability was examined to test an applicable property of codeposition. FIG. 56 shows a water droplet analyzed to determine wettability. This is a Si surface on which codeposition was performed with 500 eV Ar⁺ to a fluence of 1E17 ions/cm² with a flux ratio of 1.0. Ten images were taken of this droplet, with angular measurements taken on both sides in each image, resulting in a calculated contact angle of 80.20±2.49°, indicating a slightly hydrophilic surface.

Contact angle data on Si is shown in FIGS. 57 and 58. As a base for comparison, the contact angle on a virgin Si sample was measured to be 73.58±0.89°. To more clearly see the data sets, the Ar⁺ and O₂⁺ data are presented separately. The Ar⁺ in FIG. 57 shows a fairly narrow range of contact angles, none of which are drastically hydrophilic or hydrophobic. The 500 eV data shows an increase in contact angle (more hydrophobic) with an increase in flux ratio, while the 1000 eV results are inconsistent.

The O₂⁺ data in FIG. 58 also shows a general increase in contact angle with flux ratio. Compared with the virgin sample, nearly every data point in FIG. 58 indicates a higher contact angle after codeposition. This would indicate that codeposition performed with O₂⁺ induces a measure of hydrophobicity on the surface.

The overall increase in contact angle with flux ratio draws a natural connection to the Zn concentration data. These are compared in FIG. 59. Although rough, an inverse correlation can be seen between the atomic concentration of Zn on the surface and the resultant contact angle. In addition to this connection, the effects of O, Si, and C concentrations, as well as RMS roughness, were all investigated in relation to contact angle. None showed any correlation. This analysis was also performed on PDMS, beginning with FIG. 57, showing an example contact angle measurement. Light can be seen to illuminate the transparent PDMS substrate beneath the droplet.

The flux ratio is then evaluated for the resultant contact angle for PDMS as well in FIG. 60. Here, the 500 eV O₂⁺ data again shows that the contact angle increases with increasing flux ratio, and thus lower Zn concentration. The 1000 eV data, however, is unclear, with a fairly hydrophobic sample being created at a flux ratio of 0.1, and the rest remaining constant within the range of error.

A significant result shown in FIG. 61 is the comparison to the virgin PDMS sample, and those irradiated with 500 eV and 1000 eV O₂⁺ ions without codeposition, with the codeposition samples. All three that did not undergo codeposition have a contact angle ˜104°. Almost every codeposition sample, however, has a contact angle noticeably less than this. Since FIGS. 21 and 22 show that the morphology of the irradiated PDMS without codeposition has the same wrinkle patterns as those with codeposition, yet the same contact angle as the virgin sample, this indicates that the presence of Zn on the surface plays a role in the decrease in contact angle and move toward hydrophilicity. This is also demonstrated in FIG. 62. Although no clear trend between the actual concentration of Zn and the contact angle is seen, all but two cases have a lower contact angle after codeposition.

Conclusions

Chemical analysis of codeposited Si and PDMS samples yielded interesting results. Compositional analysis of Si showed the presence of C on all samples, possibly leading to error in both the analysis of other concentrations at the surface and in the morphological evolution of the surfaces. Overall, the flux ratio was shown to control the amount of Zn deposited on the surface, with a decrease in Zn concentration with increasing flux ratio. This was expected as a higher ratio means a higher irradiating flux compared to sputter depositing flux. Increasing the flux ratio also increased the O atomic concentration in most samples, and was not shown to be sensitive to O₂⁺ vs. Ar⁺ irradiation. This is evidence that the atomic concentration of O on the surface is determined by ballistic factors and not the chemical reactivity of the impinging ion, as Ar⁺ and O₂ are close in mass. Contrary to predictions from the SRIM calculations, a higher Zn composition was not seen at higher energies.

The XPS analysis of PDMS showed that the virgin sample contained the expected stoichiometric ratios that define the PDMS monomer. This may be misleading, however, since the PDMS samples had the same risk of atmospheric contamination as the Si. Again, a decrease in Zn concentration with increasing flux ratio was observed, as well as overall lower Zn concentrations compared with Si. The O atomic concentrations were approximately the same order of magnitude as on Si and also showed a vaguely increasing trend with flux ratio. Increased fluence showed a decrease in Zn concentration and increase in O concentration at both 500 eV and 1000 eV.

The surface wettability of Si showed an increasing trend with increasing flux ratio, making a connection to the Zn concentration. Among all samples, an increase in Zn concentration is shown to result in a decrease in contact angle, or a push toward hydrophilicity. Though this trend is shown, the most hydrophilic sample had an average contact angle of 60.49±4.28°, which is not drastically hydrophilic. The PDMS samples did not show a clear trend in change in contact angle related to the flux ratio or the Zn concentration. The codeposited PDMS samples did, however, show more hydrophilic than the virgin sample and the irradiated, but not codeposited, PDMS samples. Since these were shown to form the same wrinkle pattern, the decrease in contact angle is likely due to the presence of Zn. Since dots formed on some, but not all samples, and the dots that did form are very low profile, this is likely a chemical and not morphological effect. Also, it is possible that a trend linking the contact angle with Zn concentration is not seen due to the relatively low atomic concentration of Zn on all samples and that a trend may be observed with even higher deposition rates/concentration.

This work as evaluated codeposition as a method of synthesizing ZnO nanostructures on Si and PDMS. The effects of flux ratio, ion energy, irradiation fluence, and ion species were studied. Analysis techniques include AFM (surface morphology), XPS (surface chemistry), and contact angle (wettability). Surface morphology on Si showed the controlled creation of disordered dots ˜20-50 nm in diameter. In terms of the flux ratio, the highest (2.0) and lowest (0.1) values tend to result in little or no dot formation. Among the rest of the values, a flux ratio of 0.5 resulted in consistently well-formed dots on the surface. The energy with which codeposition was performed was also shown to have a notable impact, with higher energy (1000 eV) tending to form larger, more densely packed dots than the lower energy (500 eV). Finally, the total fluence showed two results. Dots were shown to form with 500 eV O₂⁺ codeposition at 5E17 ions/cm², as well as 1000 eV O₂⁺ codeposition at both 1E17 ions/cm² and 5E17 ions/cm². At the higher fluences, however, ripples were also seen, as expected by the normal incidence irradiation of Si with an impurity.

The codeposition work on PDMS also demonstrated the ability to create complex surfaces that integrate ZnO with the substrate. First, normal incidence irradiation was shown to create wrinkle patterns on the surface at both 500 eV and 1000 eV Ar⁺ irradiation. These patterns are similar to those seen before created by O₂⁺ plasma immersion. Like Si, a variety of dots were shown to form on the substrate during codeposition. These were larger than on Si, with diameters ˜75-200 nm. The ability to form and control these dots was less clear than on Si, likely due to the complex chemistry of the polymer. In one case, the fluence was shown to affect their formation, with a relatively high density of well-formed dots occurring at a flux ratio of 0.2, then a lesser density of the same at 0.5, then no dots at a ratio of 1.0 for codeposition with 500 eV O₂⁺. Energy did not demonstrate a significant effect on surface morphology, with dots forming on some 500 eV and 1000 eV samples, and not on others. Fluence also did not demonstrate a clear pattern, though a higher fluence of 5E17 ions/cm² did show dot formation that was not seen at 1E17 ions/cm² in some cases. One of these samples did have the interesting result of different surface morphologies at different locations, which was not seen in other samples. FIG. 43C shows that at one location on the 1000 eV O₂⁺ sample there is a very rough surface, while at another location there are bimodal small and large dots, indicating that a fine control over deposition is required to control the creation of dots. Most notably, in the case of small dot formation on PDMS, they appear to form randomly over the surface. When larger dots are seen, however, they show a high preference for the valleys of the larger wrinkle pattern. It is theorized that it is energetically preferable for the deposited material to coalesce in the valleys.

Chemical analysis showed that, for both Si and PDMS, there is a decrease in Zn concentration with increasing flux ratio, as predicted. There is also a notably lower Zn concentration on PDMS compared to Si. The O concentration for both is shown to increase with flux ratio. Interestingly, Ar⁺ and O₂⁺ did not demonstrate a significant difference in the surface morphology or chemistry, suggesting that ballistic effects of the impacting ion are more significant than chemical ones. Contact angle measurements were also performed. While observed changes were not drastic, a trend was observed. On Si, increasing the flux ratio, and thereby decreasing the Zn concentration, led to more hydrophilic surfaces. On PDMS, this trend with the flux ratio was not observed, but nearly all samples with Zn from codeposition were also more hydrophilic than the virgin material. Additionally, there was almost no change in the wettability of PDMS that was irradiated, creating the wrinkle patterns, without codeposition compared to the virgin sample. This is further evidence that the changes in wettability are due to the presence of ZnO.

Further testing is required to evaluate the usefulness of these surfaces for applications. The wettability study, however, did show that this property is controllable. Overall, the ability to controllably synthesize ZnO nanostructures on both Si and PDMS was successfully demonstrated.

This work provides the opportunity to expand on the understanding of codeposition with metal oxides and polymer substrates in a number of ways, the most significant of which is in-situ or, ideally, in-operando chemical analysis. The presence of C on the Si samples is troubling as it may have significantly altered the results. It is impossible to tell with the present technique if the C was present during the experiment, or if it became attached to the surface between the experiment and analysis. In-situ XPS would prevent any possible atmospheric contamination between the experiment and analysis, while in-operando XPS would allow the surface chemistry to be monitored in real time, allowing for the comparison of changes in the peaks relative to each other.

Additional chemical analysis techniques could also clarify the results. A more surface sensitive technique, like ion scattering spectroscopy, would probe the first monolayer and provide an interesting comparison to the XPS results. The addition of X-Ray Diffraction (XRD) could also illuminate the crystal structure of the surface and confirm the creation and phase of ZnO, and not just the deposition of Zn.

While the results of wettability are interesting and relevant, there are many other techniques that should be performed to evaluate the practical use of these materials. These include biological, mechanical, and electronic testing. Biological testing needs to be performed to determine both how the body and bacteria would react to these surfaces. Mechanical, as well as thermal and chemical, testing should be performed to evaluate the stability of the materials in extreme environments. Finally, for applications as sensors, the electronic properties of the surfaces should be evaluated and their sensitivity to different molecules.

Finally, while ZnO and PDMS are very promising biomaterials, there are certainly others worth investigating. Additional metal oxides, such as TiO₂ and MgO, are of great interest in the biomedical community and it would be worth knowing if they behave similarly. As the world of flexible biomaterials and electronics is still in its infancy, there are a number of other flexible substrates that are being investigated, including indium tin oxide (ITO) and sustainable materials like bacterial nanocellulose (BNC) and chitosan.

### REFERENCES

[1] B. P. Yang, H. Yan, S. Mao, R. Russo, J. Johnson, R. Saykally, N. Morris, J. Pham, R. He, and H. Choi, Adv. Funct. Mater. 12, 323 (2002).
[2] R. E. Mohan, M. Sivakumar, and K. S. Sreelatha, Int. J. Mod. Nonlinear Theory Appl. 1, 130 (2012).
[3] R. Knenkamp, R. C. Word, and C. Schlegel, Appl. Phys. Lett. 85, 6004 (2004).
[4] Y. Ito, K. Kushida, K. Sugawara, H. Takeuchi, and S. Member, IEEE Trans. Ultrason. Ferroelectr. Freq. Control 42, 316 (1995).
[5] S. Trolier-Mckinstry and P. Muralt, J. Electroceramics 12, 7 (2004).
[6] K. Nomura, M. Ohta, K. Ueda, T. Kamiya, M. Hirano, and H. Hosono, Science (80-.). 300, 1269 (2003).
[7] H. Harima, J. Phys. Condens. Matter 16, S5653 (2004).
[8] S. J. Pearton, W. H. Heo, M. Ivill, D. P. Norton, and T. Steiner, Semicond. Sci. Technol. 19, R59 (2004).
[9] S. PEARTON, Prog. Mater. Sci. 50, 293 (2005).
[10] M. Wagh, L. Patil, T. Seth, and D. Amalnerkar, Mater. Chem. Phys. 84, 228 (2004).
[11] Y. Ushio, Sensors Actuators B 17, 221 (1994).
[12] Z. L. Wang, X. Y. Kong, Y. Ding, P. Gao, W. L. Hughes, R. Yang, and Y. Zhang, Adv. Funct. Mater. 14, 943 (2004).
[13] Y. LIU, Y. YANG, H. YANG, Z. LIU, G. SHEN, and R. YU, J. Inorg. Biochem. 99, 2046 (2005).
[14] A. Wei, X. W. Sun, J. X. Wang, Y. Lei, X. P. Cai, C. M. Li, Z. L. Dong, and W. Huang, Appl. Phys. Lett. 89, 123902 (2006).
[15] A. El-Ansary and L. M. Faddah, Nanotechnol. Sci. Appl. 3, 65 (2010).
[16] Z. Fan and J. G. Lu, J. Nanosci. Nanotechnol. 5, 1561 (2005).
[17] U. O\ddot{z}gür, Y. I. Alivov, C. Liu, A. Teke, M. a. Reshchikov, S. Dog\check{a}n, V. Avrutin, S.-J. Cho, and H. Morkoc, J. Appl. Phys. 98, 41301 (2005).
[18] I. Mihailova, V. Gerbreders, E. Tamanis, E. Sledevskis, R. Viter, and P. Sarajevs, J. Non. Cryst. Solids 377, 212 (2013).
[19] A. P. Rambu, V. Tiron, V. Nica, and N. Iftimie, J. Appl. Phys. 113, 234506 (2013).
[20] Q. Xu, Q. Cheng, Z. Zhang, R. Hong, X. Chen, Z. Wu, and F. Zhang, J. Alloys Compd. 590, 260 (2014).
[21] J. W. Shin, J. Y. Lee, Y. S. No, T. W. Kim, and W. K. Choi, J. Appl. Phys. 100, 13526 (2006).
[22] M. R. Khanlary, V. Vahedi, and A. Reyhani, Molecules 17, 5021 (2012).
[23] S. Xu and Z. L. Wang, Nano Res. 4, 1013 (2011).
[24] K. J. Yu, D. Kuzum, S. Hwang, B. H. Kim, H. Juul, N. H. Kim, S. M. Won, K. Chiang, M. Trumpis, A. G. Richardson, H. Cheng, H. Fang, M. Thompson, H. Bink, D. Talos, K. J. Seo, H. N. Lee, S. Kang, J. Kim, J. Y. Lee, Y. Huang, F. E. Jensen, M. A. Dichter, T. H. Lucas, J. Viventi, B. Litt, and J. A. Rogers, Nat. Mater. 15, 782 (2016).
[25] S. Kang, R. K. J. Murphy, S. Hwang, S. M. Lee, D. V. Harburg, N. A. Krueger, J. Shin, P. Gamble, H. Cheng, S. Yu, Z. Liu, J. G. McCall, M. Stephen, H. Ying, J. Kim, G. Park, R. C. Webb, C. H. Lee, S. Chung, D. S. Wie, A. D. Gujar, B. Vemulapalli, A. H. Kim, K.-M. Lee, J. Cheng, Y. Huang, S. H. Lee, P. V. Braun, W. Z. Ray, and J. A. Rogers, Nature 530, 71 (2016).
[26] C. K. Choi, Sensors 8, 3257 (2008).
[27] C. M. Yang, M. H. Hon, and I. C. Leu, Ceram. Int. 38, 4277 (2012).
[28] S. Gomathi, N, Sureshkumar, A, Neogi, Curr. Sci. 94, 1478 (2008).
[29] R. J. Moon, A. Martini, J. Nairn, J. Simonsen, and J. Youngblood, Chem. Soc. Rev. 40, 3941 (2011).
[30] W. K. Czaja, D. J. Young, M. Kawecki, and R. M. Brown, Biomacromolecules 8, 1 (2007).
[31] I. Siro and D. Placket (Cellulose 17, 459 (2010).
[32] M. K. Kim, D. K. Yi, and U. Paik, Langmuir 26, 7552 (2010).
[33] I.-S. Hwang, Y.-S. Kim, S.-J. Kim, B.-K. Ju, and J.-H. Lee, Sensors Actuators B Chem. 136, 224 (2009).
[34] P. Du, X. Lin, and X. Zhang, J. Phys. D. Appl. Phys. 46, 195303 (2013).
[35] J. C. Lotters, W. Olthuis, P. H. Veltink, and P. Bergveld, J. Micromechanics Microengineering 7, 145 (1997).
[36] Dow-Corning, SYLGARD® 184 SILICONE ELASTOMER KIT (2014).
[37] L. Tsai, W. Dahlquist, S. Kom, and G. Nordin, J. Micro/Nanolithography, MEMS, MOEMS 10, (2011).
[38] J. L. Tan, J. Tien, D. M. Pirone, D. S. Gray, K. Bhadriraju, and C. S. Chen, Proc. Natl. Acad. Sci. 100, 1484 (2003).
[39] P. E. F. Neys, I. F. J. Vankelecom, M. L′abbe, R. F. Parton, and E. Cenlemans, J. Mol. Catal. A Chem. 134, 209 (1998).
[40] F. Abbasi, H. Mirzadeh, and A. Katbab, Polym. Int. 50, 1279 (2001).
[41] M. Khoo and C. Liu, Sensors Actuators A 89, 259 (2001).
[42] H. Hillborg, J. F. Ankner, U. W. Gedde, G. D. Smith, H. K. Yasuda, and K. Wikstro, Polymer (Guildf). 41, 6851 (2000).
[43] J. Duan, X. Liu, Q. Han, and X. Wang, J. Alloys Compd. 509, 9255 (2011).
[44] B.-R. Li, C.-W. Chen, W.-L. Yang, T.-Y. Lin, C.-Y. Pan, and Y.-T. Chen, Biosens. Bioelectron. 45, 252 (2013).
[45] C. J. Lee, C. H. Hsieh, H. S. Huang, and J. C. Huang, Scr. Mater. 69, 5 (2013).
[46] P.-H. Lei, H.-M. Wu, and C.-M. Hsu, Surf. Coatings Technol. 206, 3258 (2012).
[47] H. Denver, T. Heiman, E. Martin, A. Gupta, and D.-A. Borca-Tasciuc, J. Appl. Phys. 106, 64909 (2009).
[48] J. M. E. Harper and J. J. Cuomo, Annu. Rev. Mater. Sci. 13, 413 (1983).
[49] J. P. Allain, M. Nieto, M. R. Hendricks, P. Plotkin, S. S. Harilal, and A. Hassanein, Rev. Sci. Instrum. 78, 113105 (2007).
[50] F. A. Smidt, Int. Mater. Rev. 35, 61 (1990).
[51] H. R. Kaufman, J. Vac. Sci. Technol. 21, 725 (1982).
[52] J. M. E. Harper, J. Vac. Sci. Technol. 21, 737 (1982).
[53] M. Nastasi, J. Mayer, and J. Hirvonen, Ion-Solid Interactions: Fundamentals and Applications, 1st ed. (Cambridge University Press, 1996).
[54] W. D. Callister, Materials Science and Engineering: An Introduction, 7th ed. (John Wiley & Sons, Inc., 2007).
[55] P. Sigmund and N. Q. Lam, K. Danske Vidensk. Selsk. Det, Metematisk-Fysiske Meddelelser 255 (1993).
[56] P. Sigmund, J. Mater. Sci. 8, 1545 (1973).
[57] C. S. Madi, E. Anzenberg, K. F. Ludwig, and M. J. Aziz, Phys. Rev. Lett. 106, 1 (2011).
[58] Z. Yang, M. Lively, and J. P. Allain, Nucl. Instruments Methods Phys. Res. Sect. B Beam Interact with Mater. Atoms 307, 189 (2013).
[59] S. A. Mollick, D. Ghose, P. D. Shipman, and R. Mark Bradley, Appl. Phys. Lett. 104, 1 (2014).
[60] J. G. Han, J. Phys. D. Appl. Phys. 42, 43001 (2009).
[61] K. Vad, A. Csik, and G. Langer, SpectroscopyEurope 21, 13 (2009).
[62] H. Oechsner, Int. J. Mass Spectrom. Ion Process. 143, 271 (1995).
[63] M. Navez, C. Sella, and D. Chaperot, Colloq. Intern. Cent. Natl. Rech. Sci 254, 240 (1962).
[64] S. A. Mollick and D. Chose, in Proceeding Int. Conf. Recent Trends Appl. Phys. Mater. Sci. (2013), pp. 341-342.
[65] S. A. Mollick and D. Chose, J. Appl. Phys. 106, 1 (2009).
[66] R. Gago, L. Vázquez, O. Plantevin, J. A. Sanchez-Garcia, M. Varela, M. C. Ballesteros, J. M. Albella, and T. H. Metzger, Phys. Rev. B 73, 155414 (2006).
[67] G. Ozaydin, K. F. Ludwig, H. Zhou, L. Zhou, and R. L. Headrick, J. Appl. Phys. 103, 33512 (2008).
[68] C. S. Madi, B. Davidovitch, H. B. George, S. A. Norris, M. P. Brenner, and M. J. Aziz, Phys. Rev. Lett. 101, 246102 (2008).
[69] B. Ziberi, F. Frost, T. Hoche, and B. Rauschenbach, Vacuum 81, 155 (2006).
[70] J. A. Sanchez-Garcia, L. Vazquez, R. Gago, A. Redondo-Cubero, J. M. Albella, and Z. Czigany, Nanotechnology 19, 355306 (2008).
[71] G. Carter and V. Vishnyakov, Phys. Rev. B 54, 17647 (1996).
[72] J. Zhou, S. Facsko, M. Lu, and W. Möller, J. Appl. Phys. 109, 104315 (2011).
[73] K. Zhang, M. Brotzmann, and H. Hofsäss, AIP Adv. 2, 0 (2012).
[74] H. Hofsass, K. Zhang, A. Pape, O. Bobes, and M. Brötzmann, Appl. Phys. A Mater. Sci. Process. 111, 653 (2013).
[75] R. M. Bradley, J. Vac. Sci. Technol. A Vacuum, Surfaces, Film. 6, 2390 (1988).
[76] J. M. Hyman and B. Nicolaenko, Phys. D Nonlinear Phenom. 18, 113 (1986).
[77] A. Keller and S. Facsko, Materials (Basel). 3, 4811 (2010).
[78] M. Kutsuna, P. Ghosh, M. Kudo, M. Tanemura, and Y. Hayashi, Appl. Surf. Sci. 256, 1481 (2009).
[79] L.-C. Chao, C.-C. Liau, J.-W. Lee, and F.-C. Tsai, J. Vac. Sci. Technol. B Microelectron. Nanom. Struct. 25, 2168 (2007).
[80] N. V. Alov, Nucl. Instruments Methods Phys. Res. Sect. B Beam Interact with Mater.
Atoms 256, 337 (2007).
[81] N. V. Alov, J. Anal. Chem. 60, 431 (2005).
[82] J. M. E. Harper, J. Appl. Phys. 52, 4118 (1981).
[83] A. W. Kleinsasser, J. M. E. Harper, J. J. Cuomo, M. Heiblum, and Y. Heights, Thin Solid Films 95, 333 (1982).
[84] C. Oehr, Nucl. Instruments Methods Phys. Res. Sect. B Beam Interact. with Mater. Atoms 208, 40 (2003).
[85] C.-M. Chan, T.-M. Ko, and H. Hiraoka, Surf. Sci. Rep. 24, 1 (1996).
[86] C. Yanling, W. Yingkuan, P. Chen, S. Deng, and R. Ruan, 7, 1 (2014).
[87] D. B. H. Chua, H. T. Ng, and S. F. Y. Li, Appl. Phys. Lett. 76, 721 (2000).
[88] A. Tserepi, E. Gogolides, K. Tsougeni, V. Constantoudis, and E. S. Valamontes, J. Appl. Phys. 98, 1 (2005).
[89] P. Karmakar and D. Chose, Surf. Sci. 554, L101 (2004).
[90] D. Chose, J. Phys. Condens. Matter 21, 224001 (2009).
[91] A. Ide-Ektessabi, H. Nomura, N. Yasui, and Y. Tsukuda, Thin Solid Films 447-448, 383 (2004).
[92] P. Gailly, C. Petermann, P. Tihon, and K. Fleury-Frenette, Appl. Surf. Sci. 258, 7717 (2012).
[93] M. Stepanova and S. K. Dew, J. Phys. Condens. Matter 21, 224014 (2009).
[94] F. Buatier de Mongeot and U. Valbusa, J. Phys. Condens. Matter 21, 224022 (2009).
[95] N. Vourdas, A. Tserepi, and E. Gogolides, Nanotechnology 18, 125304 (2007).
[96] C. N. Taylor, K. E. Luitjohan, B. Heim, L. Kollar, J. P. Allain, C. H. Skinner, H. W. Kugel, R. Kaita, a. L. Roquemore, and R. Maingi, Fusion Eng. Des. (2013).
[97] N. Alov, D. Kutsko, I. Spirovová, and Z. Bastl, Surf. Sci. 600, 1628 (2006).
[98] M. Joe, J.-H. Kim, C. Choi, B. Kahng, and J.-S. Kim, J. Phys. Condens. Matter 21, 224011 (2009).
[99] M. Joe, C. Choi, B. Kahng, and J.-S. Kim, Appl. Phys. Lett. 91, 233115 (2007).
[100] J. M. E. Harper, J. Vac. Sci. Technol. 16, 1901 (1979).
[101] Tectra GmbH Physikalische Instrumente, Plasma Source Operating Manual: Genii Plasma Source (Frankfurt, 2012).
[102] I. Horcas, R. Fernández, J. M. Gómez-Rodriguez, J. Colchero, J. Gómez-Herrero, and A. M. Baro, Rev. Sci. Instrum. 78, (2007).
[103] E. R. Cawthron, Aust. J. Phys. 24, 859 (1971).
[104] J. G. Martel and N. T. Olson, Nucl. Instruments Methods 105, 269 (2000).
[105] C. Hopf, A. von Keudell, and W. Jacob, J. Appl. Phys. 94, 2373 (2003).
[106] S. Wu, Polymer Interface and Adhesion, 1st ed. (M. Dekker, New York, 1982).

### Example 2: Directed Plasma Nanosynthesis of Multi-Functional Natural Nanocellulose Interfaces with Anti-Bacterial and Super-Hydrophilic Properties

Metal nanoparticles have attracted much attention for their unusual chemical and physical properties. Gold nanoparticles have been used in many fields such as biotechnology, optics, electronics, catalysis, and sensors. [1-3] Silver nanoparticles have also been widely used in sensors, antibacterial and photocatalytic areas. [4-6] The synthesis of nanoparticles with different chemical composition, size distribution, and controlled mono dispersion is an important area of research in nanotechnology. Many methods such as vapor deposition, solvent-thermal, sol-gel, electrochemistry and microwave have been developed to fabricate nanoparticles. [1-8] The stability and functional properties of nanoparticles are critical to their application, which are traditionally determined by the coatings. Bacterial nanocellulose (BNC) and chitosan (CS) are fascinating and renewable natural nanomaterial characterized by favorable properties such as remarkable mechanical properties, porosity, water absorbency, moldability, biodegradability and excellent biological affinity. Intensive research and exploration in the past few decades on BNC/CS nanomaterials mainly focused on their biosynthetic process to achieve the low cost preparation and application in medical, food, advanced acoustic diaphragms, and other fields. These investigations have led to the emergence of more diverse potential applications exploiting the functionality of BNC/CS nanomaterials. There is a great demand for multifunctional nanomaterials in the biomedical, new energy and other areas. Recently, Ag or Au nanoparticle-modified BNC have been fabricated using traditional chemical methods and exploited their application in antibacterial, detector, sensor, catalysis, and imaging [9-20].

Many methods such as vapor deposition, solvent-thermal, sol-gel, co-deposition have been widely used to fabricate nanoparticles. In order to form the nanomaterials and nanostructure, many chemical and complex reaction conditions are needed. The extensive use of chemicals will increase the content of impurity in products, produce environmental pollution and increase the cost. Harsh conditions are needed to induce reactions in traditional chemical synthesis methods, which will undergo complex process and need expensive equipment. The structures of the reactant especially some bioactive materials and natural polymers are easily destroyed in high temperature, high pressure and solvent chemical environments. Therefore, simple, high efficiency and low cost synthesis methods under ambient temperature and pressures are needed. Ion irradiation can produce many kinds of active particles and can be used to fabricate materials and surfaces.

The invention consists of a method of extracting an energetic beam of ions or a combination of radicals and ions to irradiate a natural biomaterial surface pre-treated in a chemical metal-based solution to create two critical functions: 1) induce nanoparticle synthesis and integration into the natural biomaterial nanocellulose surface and 2) transform the interface into an anti-bacterial, bactericidal surface capable of combating disease while maintaining a superhydrophilic surface to enable protein adhesion and enhanced bioactive properties (e.g., biosensing, biocompatibility, tissue reconstruction, etc. The method of beam extraction is dictated by the energy density required to balance the material response rate with the irradiation stimulant rate. One of the greatest chasms in multi-functional biointerfaces is the ability to synthesize an interface that is both hydrophilic providing excellent cell adhesion for tissue reconstruction and healing yet preventing bacterial adhesion that could ultimately lead to infection. In addition, designing natural-based biomaterials that can integrate with the harsh chemical environment of the body is desired. The invention provides for the first time a multi-functional interface by synthesizing Ag or Au nanoparticles into the surface matrix of natural nanocellulose (e.g., bacterial nanocellulose, chitosan) and transforming its surface into an anti-bacterial nanostructure yet maintaining its hydrophilic properties. In fact in the chase of chitosan the method transforms the interface from hydrophobic to hydrophilic.

Another important purpose for this invention is the fact that the complex 3D structure engineering in this natural biomaterial in fact mimics more closely the complex 3D extreme environment of the body. With DPNS, DIS, DSDPNS, or DSPNS one can then systematically tailor the surface chemistry, topology, morphology, surface energy, surface charge density and thus provide for intelligent biomimetic hydrogel scaffolds for tissue engineering and regenerative medicine applications. Most in-vitro studies are carried out in 2D cultures testing various drug delivery or tissue reconstruction techniques. However, in-vivo cells and tissues are immersed in a complex hierarchy of anno to microscale porous topography. Directing cell behavior via the complex structural and biochemical environment in the body and studying this in vitro is one of the most pressing challenges in the field of regenerative medicine and tissue engineering.

The invention is an unprecedented biointerface that is both anti-bacterial and hydrophilic providing for an ideal tissue engineering material. The use of BNC and chitosan enable the design of devices such as membranes that could be used with stents in blood vessels for repair of damaged vascular lesions induced by hemodynamic activity (e.g., cardiovascular systems, cerebrovascular aneurysms, abdominal aneurysms, etc.). Broader applications include biosensors that require Ag or Au nanoparticles embedded in natural biomaterials for use with surface-enhanced Raman spectroscopy (SERS) where the sensitivity is enhanced by the DPNS, DIS, DSDPNS, or DSPNS-provided nanostructuring of the surface. The incorporation of Au and Ag nanoparticles also enhances the intrinsic anti-bacterial properties of the nanostructured natural nanocellulose.

The key to the invention is also the DPNS, DIS, DSDPNS, or DSPNS process of the natural nanocellulose does not involve any toxic or high-temperature treatment, which reduces chemical waste and enhances the ability to introduce nanostructuring without costly thermal cycling or houses-days of curing steps.

FIG. 63A-B shows results of irradiation synthesis of nanostructured natural nanocellulose materials with enhanced multi-functional properties. FIG. 63A-B shows before/after SEM images (low and high resolution) of chitosan or bacterial nanocellulose material synthesized with Au nanoparticles and Ag nanoparticles irradiated with DPNS at 1-keV Ar⁺ normal incidence. FIG. 64 shows results of irradiation synthesis of nanostructured natural nanocellulose materials with enhanced multi-functional properties. X-ray diffraction data showing enhanced peak from embedded metal nanoparticles in the irradiated matrix. FIG. 65 shows results of irradiation synthesis of nanostructured natural nanocellulose materials with enhanced multi-functional properties. FIG. 65 shows contact angle data for chitosan samples with corresponding diagram to illustrate the transition of chitosan from hydrophobic to hydrophilic properties after irradiation at room temperature. FIG. 66 shows results of irradiation synthesis of nanostructured natural nanocellulose materials with enhanced multi-functional properties. FIG. 66 shows SEM before/after of bacterial nanocellulose integrated with Ag nanoparticles. Note the dramatic transformation from BNC cellulose to pillar super nanostructures. FIG. 67 shows results of irradiation synthesis of nanostructured natural nanocellulose materials with enhanced multi-functional properties. FIG. 67 shows contact angle data for BNC samples with corresponding diagram.

FIG. 68 shows a diagram of sequence of synthesis of natural cellulose irradiated by DPNS. Fabrication of the pure chitosan (CS) film: 600 μL of HCl is placed into 60 mL of water and stirred to form a 1% HAc solution. 0.5 g of CS (available commercially) in placed in the above solution. The solution is stirred magnetically and ultrasonically dispersed. After a solution forms, 10-15 mL of the CS solution into a 5.5 cm culture dish. This is placed into an oven (37-40° C.) for 1-2 days until a dried film is formed.

Fabrication of Ag, Au/CS film: 0.2 mL of AgNO₃ or HAuCl₄ solution (100 mM) in 10 mL was added to the CS solution (10-15 mL) prepared as above. The solution is stirred for 20 minutes. This is placed into an oven (37-40° C.) for 1-2 days until a dried film is formed.

Fabrication of Ag, Au/BNC film. Preparation of bacterial nanocellulose. BNC is prepared as known in the art. Basically, BNC is prepared as follows. 500 mL of liquid culture medium by combining 25 g yeast extract, 15 g of peptone, 125 g of mannitol, 500 mL of high purity water. Mixture was autoclaved at 120° C. for 20 minutes and stored at 4° C. 100 mL of semisolid media was prepared by adding 15 g of agar to 5.0 g of yeast extract, 3.0 g of peptone, 25.0 g of mannitol, and 100 mL of high purity water. Mixture was autoclaved at 120° C. for 20 minutes. Once autoclaved, sposit 5 mL of the mixture in a 90 mm×16 mm plastic petri dish. Allow the solution to gel at 4° C. and store at this temperature until further use. Rehydrate G. xylinus strain preserved in freeze-dried vials by adding 1 mL of liquid culture medium and pipetting up and down, as indicated by the manufacturer's instructions. Inoculate the petri dishes containing semisolid media with small droplets of bacterial suspension using an inoculating loop. Make sure that the inoculum covers the entire petri dish by moving the loop in a zig zag direction from the edge to the center of the dish. Incubate the petri dishes at 26° C. for 72 hours in an incubator without 002. Once the incubation period is complete, small white colonies are visible. If colonies are not immediately used, store the dishes at 4° C. by sealing the lid with parafilm and placing the dishes upside down. They can be stored up to six months. Transfer 2 mL of the liquid culture medium into each well of a 24 well tissue culture plate. Take two colonies with an inoculating needle from the inoculated petri dishes and place them into the first well of the tissue culture plate. Repeat with the remaining 23 wells. Incubate the tissue culture plate at 30° C. for 7 days. This will yield a total 24 BNC pellicles with a diameter of 16 mm and a thickness of approximately 2-3 mm diameter as depicted in FIG. 73. Do not disturb the bacterial culture during the incubation period; during the incubation period, G. xylinus extrudes glycopyranose sugar molecules to form a polymeric crystalline mesh in the air-liquid interface, which adopts the shape and size of the flask under static cultivation conditions. This polymeric matrix, known as bacterial nanocellulose, is conspicuous at the end of the incubation period. The BNC pellicules are collected from the growth media and sterilized in 200 mL 1% NaOH solution for 1 hour at 50° C., in order to remove all traces of G. xylinus. Optimally stir this solution at 300 rpm using a magnetic stirbar and a stirring plate. Discard the NaOH solution and add 200 mL freshly prepared 1% NaOH solution. Repeat this process one or more times until the BNC pellicules acquire a translucent appearance. Rinse the BNC pellicules with water three times and store in high purity water at room temperature. Ensure that the pellicles are completely submerged and are not allowed to dry. Autoclave at 121° C. for 20 minutes. Prepared BNC film is placed into a 10 mM AgNO₃ or HAuCl₄ solution for 1-2 hours. This is placed into an oven (37-40° C.) for 1-2 days until a dried film is formed.

FIG. 69 shows conditions under which the materials were formed. The DPNS parameters consist of irradiation with either Ar⁺ or O₂⁺ or both extracted from an rf ECR plasma and combined at energies below 1-keV and normal incidence for fluences between 10¹⁶ and 10¹⁷ cm⁻². Plasma Jet Conditions: Information unlimited PVM500 RF Power, 20 kV, 20 kHz to 70 kHz. Alicat Scientific MC2SLPM-D Mass Flow controller, Argon gas (Ar), mass flow 2 slpm. Inner diameter of the glass tube 1 mm. Width of Cu foil 1 cm. Distance between the end of the glass tube and the Cu foil 0.5-1 cm.

### REFERENCES

[1] Xuan Yang M Y et al. (2015) “Gold Nanomaterials at Work in Biomedicine,” Chern Rev 115:10410.
[2] Wen Zhou X G et al. (2015) “Gold Nanoparticles for In Vitro Diagnostics,” Chern Rev 115:1157.
[3] Xiao Yan Liva A W et al. (2013) “Catalysis by gold: New insights into the support effect,” nano today 8:403.
[4] Benjamin Le Ouay F S (2015) “Antibacterial activity of silver nanoparticles: A surface science insight,” nano today 10:339,
[5]. Sonja Eckhardt P S B et al. (2013) “Fromm Nanobio Silver: Its Interactions with Peptides and Bacteria, and Its Uses in Medicine,” Chern Rev 113:4708.
[6] Bi GFaX (2015) “Silver-catalysed reactions of alkynes: recent advances,” Chern Soc Rev 44:8124.
[7] Francesco Meinardi H M et al. (2015) “Highly efficient large-area colourless luminescent solar concentrators using heavy-metal-free colloidal quantum dots,” Nature Nanotechnology 10:878.
[8] Maria V. Mukhina et al. (2015) “Intrinsic Chirality of CdSe/ZnS Quantum Dots and Quantum Rods,” Nano Lett 15:2844.
[9] PC Faria-Tischer et al. (2016) “Structure and effects of gold nanoparticles into bacterial cellulose-polyaniline conductive membranes,” RSC Advances.
[10] B. Heli et al. (2016) “Modulation of population density and size of silver nanoparticles embedded in bacterial cellulose via ammonia exposure: visual detection of volatile compounds in a piece of plasmonic nanopaper,” Nanoscale.
[11] Nahid Pourreza (2015) “Green in-situ synthesized silver nanoparticles embedded in bacterial cellulose nanopaper as a bionanocomposite plasmonic sensor,” Biosensors and Bioelectronics 74:353-359.
[12] Yan Ge et al. (2015) “Color-tunable luminescent CdTe quantum dot membranes based on bacterial cellulose (BC) and application in ion detection,” RSC Adv 5:55756.
[13] Nicolle Dal'Acqua (2015) “Characterization and Application of Nanostructured Films Containing Au and TiO2 Nanoparticles Supported in Bacterial Cellulose,” Phys Chem C 119:340-349.
[14] Meiyan Chen (2015) “Bacterial Cellulose Supported Gold Nanoparticles with Excellent Catalytic Properties,” ACS Appl Mater Interfaces 7:21717-21726.
[15] Ning Yan (2015) “Antibacterial properties and cytocompatibility of bio-based nanostructured carbon aerogels derived from silver nanoparticles deposited onto bacterial cellulose,” RSC Adv 5:97467.
[16] Jian Wu (2014) “In-situ synthesis of silver-nanoparticles/bacterial cellulose composites for slow-released antimicrobial wound dressing,” Carbohydrate Polymers 102:762-771.
[17] Parastoo Pourali (2014) “Impregnation of the Bacterial Cellulose Membrane with Biologically Produced Silver Nanoparticles,” Curr Microbial 69:785-793.
[18] Tao Niu (2014) “Cellulose-based catalytic membranes fabricated by deposition of gold nanoparticles on natural cellulose nanofibres,” RSC Adv 4:4901.
[19] Saionara V. Costa (2013) “ZnO nanostructures directly grown on paper and bacterial cellulose substrates without any surface modification layer,” Chem Commun.
[20] Guang Yang (2012) “Hydrothermal synthesis of bacterial cellulose/AgNPs composite: A “green” route for antibacterial application,” Carbohydrate Polymers 87:2482-2487.

### Example 3. Liquid Plasma Directed Synthesis of Nanoparticles and Multi-Functional Natural Materials

The invention comprises using a non-equilibrium liquid plasma (a.k.a. solution plasma) with natural biomaterials (e.g., chitosan, natural nanocellulose) and combine with specific liquid solutions that drive nanoparticle synthesis (e.g. Au, Ag, ZnS) and/or integration with carbon allotrope systems (e.g. graphene, graphene oxide, graphene, graphite, etc.) and creation of interface nanopatterning providing for enhanced properties including: biocompatibility, cell adhesion, biosensing, peptide and biomolecule adhesion, pharmaceutical adhesion, and payload delivery and biomarker sensitivity.

Metal nanoparticles and semiconductor nanocrystals (also known as quantum dots, QDs) have attracted much attention for their unusual chemical and physical properties. Gold nanoparticles have been used in many fields such as biotechnology, optics, electronics, catalysis and sensors [1-3]. Silver nanoparticles have also been widely used in sensors, antibacterial and photocatalytic areas [4-6]. ZnS QDs exhibit wide applications in bio-imaging, disease detecting, cancer therapy, energy transfer and so on [7-8]. The synthesis of nanoparticles with different chemical composition, size distribution, and controlled mono-dispersity is an important area of research in nanotechnology. Many methods such as vapor deposition, solvent-thermal, sol-gel, electrochemistry and microwave have been developed to fabricate nanoparticles [1-8]. The stability and functional properties of nanoparticles are critical to their application, which are traditionally determined by the coatings.

Chitosan is a kind of natural polymer and has been extensively used in drug delivery systems, gene therapy, tissue engineering, and biosensors. This is not only because of its low price, excellent biocompatibility, biodegradability, low toxicity, but also its unique cationic property and facile functionalization with various molecules due to amino and hydroxyl groups in chitosan. Carlo et al. synthesized gold-chitosan nano composite for caffeic acid sensing via an organic acids reduction method [9]. Baginskiy et al. prepared chitosan-modified stable colloidal nanostars for the photothermolysis of cancer cells [10]. Anna et al. developed noncytotoxic chitosan-gold nanocomposites as efficient antibacterial materials via chemical reduction method [11]. Tran et al. synthesized silver/chitosan nanocomposite via a hydrothermal method and studied their application in electrochemical detection of hydrogen peroxide [12]. Mironenko et al. fabricated chitosan/Au and chitosan/Ag nanocomposites used as H2S gas sensors via NaBH4 reduction method [13]. Mansur et al. fabricated nano-photocatalysts based on ZnS QDs/chitosan for the photodegradation of dye pollutants via chemical co-deposit method [14]. Huang et al. synthesized fluorescent chitosan-ZnSe/ZnS nanoparticles for potential drug carrier via three steps [15]. Chang et al. fabricated biocompatible chitosan coated ZnS and ZnS:Mn QDs via a gamma radiation route [16].

There is a great deal of demand for multifunctional nanomaterials in biomedical, new energy and other areas. For example, a nanocomplex for drug delivery will have drug molecules to therapy, have specific molecules to realize target delivery, have detectable molecules to determine their distribution. Carbon nanotubes (CNTs) and graphene have a large surface and low toxicity. So they are good carriers for multiple kinds of nanoparticles and have been widely used. Lokman et al. fabricated Au/chitosan/graphene oxide nanostructure films via a sputter method, which has high sensitive SPR response to Pb(II) ions [17]. Lin et al. fabricated silver/CNTs/chitosan film used as a glucose biosensor [18]. Ni et al. enhance the power conversion efficiency of solar cells by functional CNTs decorated with CdSe/ZnS QDs [19]. Gholivand et al. fabricated an electrochemical sensor for warfarin determination based on covalent immobilization of QDs onto CNTs and chitosan composite film [20]. Graphene can be produced via bottom-up synthesis and up-bottom exfoliation of graphene [21, 22]. Although bottom-up synthesis, such as crystallization based on vapor deposition, have made an essential contribution to the progress of research on graphene, these methods are costly and not readily scalable, and so do not meet industrial demands. In contrast, “liquid-phase” exfoliation of graphite is recognized as the most inexpensively scalable method [22, 23]. This approach usually employs fluids that have an affinity towards carbon allotrops in combination with certain mechanical agitation forces generated by sonication, shear mixing or other methods. Recently, there are many researchers focusing on the preparation of graphene.

Plasma technology has been widely used in the fabrication and reinforcement of materials and surfaces [24-27]. Atmospheric pressure plasma jet (APPJ) has become a mature technology that is suitable for industrial use. An APPJ is operated at a pressure of 1 atm in an open environment. Therefore, it is particularly useful for materials processes involving decomposed or evaporated chemicals that may severely pollute enclosed tubes or chambers. Having highly reactive and energetic species in APPJs is advantageous for biomaterial sterilization and treatment, Pt reduction, surface modification, thin film deposition, substrate cleaning, rapid annealing, rapid sintering of nanoporous TiO2 photoanodes and reduced graphene oxides (rGOs) counter electrodes for dye-sensitized solar cells, and rapid surface activation of graphite felt electrode for all vanadium redox flow batteries [28-32].

As shown above, many methods such as vapor deposition, solvent-thermal, sol-gel, codeposition have been widely used to fabricate nanoparticles [1-8]. In order to form the nanomaterials and nanostructure, many chemicals and complex reaction mixtures are needed. The extensive use of chemicals will increase the content of impurities in products, produce environmental pollution and increase the cost. Harsh conditions are needed to induce reactions in traditional chemical synthesis methods, which will undergo complex process and need expensive equipment. The structures of reactants especially some bioactive materials and natural polymers are easily destroyed in high temperature, high pressure and solvent chemical environment. Therefore, simple, high efficiency and low cost synthesis methods under ambient temperature and pressure are necessary. Plasma can produce many kinds of active particles and can be used to fabricate materials and surfaces. Comparing with solid and gas conditions, more parameters can be designed to fabricate novel nanomaterials in solution environment, which attracts more and more attention [33]. Some simple nanomaterials have been synthesized using solution plasma [29, 33-35]. It will be a challenge to fabricate complex nano materials using plasma method. There have been no reports of plasma-assisted fabrication and chitosan coated nanoparticles (Ag, Au, ZnS) and their complex with carbon nanotubes and graphene as yet. Efficient exfoliation of graphite to high quality graphene is another challenge. Solvent chemistry and ultrasonic methods have been used to fabricate graphene from graphite [22, 23]. There is no existing literature about the exfoliation of graphite under synergetic treatments of plasma and ultrasonic. Atmospheric pressure plasma jet can induce chemical reactions under mild conditions, can be realized easily and is low cost, which can result in it being widely used to fabricate and reinforce materials in the future.

The hypothesis for this experiment is to fabricate natural-based materials such as chitosan coated nanoparticles (Ag, Au, ZnS) using a liquid plasma method. Plasma can produce many active particles including reduction processes with generated oxygen radicals. Chemical measures are taken to increase the reduction rate and decrease the oxygen radical in reaction system. Ag⁺, AuCl₄⁻, and S₂O₃²⁻ can be reduced to Ag(0), Au( )) and S²⁻ to form Ag, Au and ZnS nanoparticles. Chitosan plays an important role in this experiment. First, chitosan can limit the growth of particles and form the nanoparticles. Second, chitosan can prevent the aggregation of the as-prepared nanoparticles and keep them in good dispersion. Third, chitosan is a kind of a natural polymer and allow the products to have good biocompatibility. The active particles produced by plasma can insert into the layer and break the bond between layers in flake graphite and form several layers or one-layer graphene in solution. The ultrasonic can increase the movements of active radicals and layers, which can accelerate the exfoliation of graphite. The synergetic treatments of plasma and ultrasonic in solution may be a good method to produce high quality graphene. Carbon nanotubes (CNT) and graphene have large surface and can be used to produce —COOH groups on the surface, which provide conjoint point with chitosan and nanoparticles. Chitosan, Ag⁺, AuCl₄⁻, and S₂O₃²⁻ can be directed attached of the surface of CNTs and graphene. The ions can also be attached of the chitosan on the surface of the CNTs and graphene. CNTs and graphene limit the growth of particles and prevent the aggregation of the prepared nanoparticles. The parameter of plasma and the composition of solution can be designed to form ideal noncomplex. All of the above experiments are conducted under ambient temperature and pressure.

Plasma Jet Conditions

Information unlimited PVM500 RF Power, 20 kV, 20 kHz to 70 kHz. Alicat Scientific MC2SLPM-D Mass Flow controller, Argon gas (Ar), mass flow 2 slpm. Inner diameter of the glass tube 1 mm. Width of Cu foil 1 cm. Distance between the end of the glass tube and the Cu foil 0.5-1 cm.

Fabrication of the Natural Polymer Films

Fabrication of pure chitosan (CS) film. FIG. 71 shows a diagram of sequence of synthesis of natural cellulose irradiated by DPNS. Fabrication of the pure chitosan (CS) film: 600 μL of HAc is placed into 60 mL of water and stirred to form a 1% HAc solution. 0.5 g of CS (obtained commercially) is placed in the above solution. The solution is stirred magnetically and ultrasonically dispersed. After a solution forms, 10-15 mL of the CS solution into a 5.5 cm culture dish. This is placed into an oven (37-40° C.) for 1-2 days until a dried film is formed. The CS film is taken from the dish and washed with 5% NaOH solution until pH=7. The CS film is then placed into an oven (37-40° C.) for 3-4 hours until dry.

Fabrication of pure BNC, CS with nanopatterns: the BNC or CS film (BNC prepared as disclosed in Example 2) is placed in a dish with pure water. The film is immersed under the solution. The distance between the film and solution surface is 1-5 mm. The distance between the end of the glass tube and solution surface is 0.5-1 cm. The treat time is 3-5 minutes. After plasma treatment, the film is taken out and washed 3 times with pure water. After that, the film is placed in the oven (37-40° C.) 3-4 hours until dry.

Fabrication of pure BNC, CS with nanopatterns and nanoparticles: the BNC or CS film (BNC prepared as disclosed in Example 2) is placed in a dish containing HAuCl₄ solution at 2-5 mM. The film is immersed under the solution. The distance between the film and solution surface is 1-5 mm. The distance between the end of the glass tube and solution surface is 0.5-1 cm. The treat time is 3-5 minutes. After plasma treatment, the film is taken out and washed 3 times with pure water. After that, the film is placed in the oven (37-40° C.) 3-4 hours until dry.

### REFERENCES

[1] Xuan Yang M Y et al. (2015) “Gold Nanomaterials at Work in Biomedicine,” Chem Rev 115:10410.
[2] Wen Zhou X G et al. (2015) “Gold Nanoparticles for In Vitro Diagnostics,” Chem Rev 115:1157.
[3] Xiao Yan Liva A W et al. (2013) “Catalysis by Gold: New Insights into the Support Effect,” Nano Today 8:403.
[4] Benjamin Le Ouay F S. (2015) “Antibacterial activity of silver nanoparticles: A surface science insight,” Nano Today 10:339.
[5] Sonja Eckhardt P S B et al. (2013) “Fromm Nanobio Silver: Its Interactions with Peptides and Bacteria, and Its Uses in Medicine,” Chem Rev 113:4708.
[6] Bi GFaX. (2015) “Silver-catalysed reactions of alkynes: recent advances,” Chem Soc Rev 44:8124.
[7] Francesco Meinardi H M et al. (2015) “Highly efficient large-area colourless luminescent solar concentrators using heavy-metal-free colloidal quantum dots,” Nature Nanotechnology 10:878.
[8] Maria V. Mukhina et al. (2015) “Intrinsic Chirality of CdSe/ZnS Quantum Dots and Quantum Rods,” Nano Lett 15:2844.
[9] Gabriella DiCarlo A C et al. (2012) “Green Synthesis of Gold-Chitosan Nanocomposites for Caffeic Acid Sensing,” Langmuir 28:5471.
[10] Ivan Baginskiy T-C L et al. (2013) “Chitosan-Modified Stable Colloidal Gold Nanostars for the Photothermolysis of Cancer Cells,” J Phys Chem C 115:2396.
[11] Anna Regiel-Futyra M K-L et al. (2015) “Development of Noncytotoxic Chitosan-Gold Nanocomposites as Efficient Antibacterial Materials,” ACS Appl Mater Interfaces 7:1087.
[12] Tran H V T et al. (2016) “Facile Hydrothermal Synthesis of Silver/Chitosan Nanocomposite and Application in the Electrochemical Detection of Hydrogen Peroxide,” Sensor Letters 14:32.
[13] A. Yu. Mironenko A A S et al. (2016) “H2S optical waveguide gas sensors based on chitosan/Au and chitosan/Ag nanocomposites,” Sensors and Actuators B: Chemical 225:348.
[14] Alexandra A. P. Mansura H S M et al. (2014) “‘Green’ colloidal ZnS quantum dots/chitosan nano-photocatalysts for advanced oxidation processes: Study of the photodegradation of organic dye pollutants,” Applied Catalysis B: Environmental 269.
[15] Liying Huang Y L et al. (2015) “Synthesis and characterization of fluorescent ChitosanZnSe/ZnS nanoparticles for potential drug carrier,” RSC Adv DOI: 10.1039/C5RA02933C.
[16] Shu-quan Chang B K et al. (2011) “One-step fabrication of biocompatible chitosan-coated ZnS and ZnS:Mn2+ quantum dots via a y-radiation route,” Nanoscale Res Lett 6:591.
[17] Nurul Fariha Lokman A A A B et al. (2014) “Highly sensitive SPR response of Au/chitosan/graphene oxide nanostructured thin films toward Pb (II) ions,” Sensors and Actuators B: Chemical 195:459.
[18] Jiehua Lin C H et al. (2009) “One-step synthesis of silver nanoparticles/carbon nanotubes/chitosan film and its application in glucose biosensor,” Sensors and Actuators B: Chemical 137:768.
[19] Ting Ni J Y et al. (2014) “Enhancement of the power conversion efficiency of polymer solar cells by functionalized single-walled carbon nanotubes decorated with CdSe/ZnS core-shell colloidal quantum dots,” Journal of Materials Science 49:2571.
[20] Mohammad Bagher Gholivand L M-B (2015) “An electrochemical sensor for warfarin determination based on covalent immobilization of quantum dots onto carboxylated multiwalled carbon nanotubes and chitosan composite film modified electrode,” Materials Science and Engineering: C 57:77.
[21] Michio Matsumoto Y S et al. (2015) “Ultrahigh-throughput exfoliation of graphite into pristine ‘single-layer’ graphene using microwaves and molecularly engineered ionic liquids,” Nature Chemistry 7:730.
[22] Keith R. Paton E V et al. (2014) “Scalable production of large quantities of defect-free fewlayer graphene by shear exfoliation in liquids,” Nature Materials 13:624.
[23] Yenny Hernandez V N et al. (2008) “High-yield production of graphene by liquid-phase exfoliation of graphite,” Nature Nanotechnology 3:536.
[24] Jingyuan Jia S K J et al. (2015) “Plasma-Treated Thickness-Controlled Two-Dimensional Black Phosphorus and Its Electronic Transport Properties,” ACS Nano 9:8729.
[25] Erik C. Neyts K K O et al. (2015) “Plasma Catalysis: Synergistic Effects at the Nanoscale,” Chern Rev 115:13408.
[26] Neeraj Dwivedi R J Y et al. (2015) “Understanding the Role of Nitrogen in Plasma-Assisted Surface Modification of Magnetic Recording Media with and without Ultrathin Carbon Overcoats,” Scientific Reports. 5:7772.
[27] Igor Levchenko M K et al. (2015) “Plasma treatment for next-generation nanobiointerfaces. Biointerphases,” 10:029405.
[28] Paul Rumbach D M B et al. (2015) “The solvation of electrons by an atmospheric-pressure plasma,” Nature Communications 6:7426.
[29] Liangliang Lin Q W (2015) “Microplasma: A New Generation of Technology for Functional Nanomaterial Synthesis,” Plasma Chemistry and Plasma Processing 35:925.
[30] Jian-Zhang Chen W-Y L et al. (2015) “All-vanadium redox flow batteries with graphite felt electrodes treated by atmospheric pressure plasma jets,” Journal of Power Sources 274:894.
[31] Hsiao-Wei Liu S-pL et al. (2014) “Rapid Atmospheric Pressure Plasma Jet Processed Reduced Graphene Oxide Counter Electrodes for Dye-Sensitized Solar Cell,” ACS Appl Mater Interfaces 6:15105.
[32] Chia-Yun Chou H C et al. (2015) “Atmospheric-pressure-plasma-jet processed nanoporous TiO2 photoanodes and Pt counter-electrodes for dye-sensitized solar cells,” RSC Adv. 5:45662.
[33] Akiyama GSaT (2015) “Nanomaterial Synthesis Using Plasma Generation in Liquid,” Journal of Nanomaterials 2015:123696.
[34] Ting-Jui Wu C-Y C et al. (2015) “Ultrafast synthesis of continuous Au thin films from chloroauric acid solution using an atmospheric pressure plasma jet,” RSC Adv 5:99654.
[35] Mathieu Bouchard M L et al. (2015) “Rapid Nucleation of Iron Oxide Nanoclusters in Aqueous Solution by Plasma Electrochemistry,” Langmuir 31:7366.

### Example 4. Surface Modification of Silk Structures

Long gap peripheral nerve injuries have high incidence worldwide, with important repercussion for the patient (pain, unlikely recovery, limited functionality). Due to the current limitations of the nerve graftings, as a standard treatment, synthetic and natural biomaterials have been used to prepare artificial Nerve Guidance Conduits (NGC) for nerve reconstruction [1]. The limited success of these strategies has determined the search for alternatives. An interesting approach to increase their bioactivity is to focus on nanopatterning functionalization. Several authors pointed to different aspects that should be taken into account, highlighting the essential role that topographical cues play in neurite outgrowth [2]). In addition, the presence of ordered channeled nanostructures has proved to facilitate guided nerve elongation. This is probably because cell alignment plays an important role during the regeneration of the damaged nerves. Surface modification techniques can promote or prevent the adhesion of biological molecules and cells to the biomaterial, while the bulk of properties and functionality of the substrate are not affected. Directed plasma nanosynthesis (DPNS) has demonstrated to induce controlled chemical and physical surface modifications at the nanoscale level, which will enhance nerve regeneration.

Directed plasma nanosynthesis (DPNS) has demonstrated to induce controlled chemical and physical surface modifications at the nanoscale level, which will guide the elongation required to cover the long gap which finally allow the reconstruction of the damaged nerve. When combining the neutral and reactive beams at energies between 50-1000 eV with multi-plexing at the surface, DPNS is the governing structuring mechanism. When the reactive beam is in the hyperthermal regime or energies of 0.1-10 eV, then we define it as DSPNS (directed soft plasma nanosynthesis).

Different alternatives based on biomaterials have been previously studied, including keratin, chitosan, alginate, gellan gun, collagen, fibroin, laminin, hyaluronic acid and silk fibroin. The latter has attracted attention due to its biocompatibility, biodegradability and tailorable mechanical properties [3].

3D tubular structures of silk has been performed and evaluated as suitable templates to improved nerve regeneration. The main focus of these research study is inducing different cell behavior by developing surface modification at the nano scale order inside and outside in a selective and controlled manner, using DPNS to modified the surface and functionalized them with growth factors and peptides.

Our main goal is to reproduce the nerve extracellular matrix introducing nanofeatures with adsorbed growth factors in order to promote cell adhesion and proliferation within the tube to protect the nerve injured as well regenerate the gap in this class of damaged nerve tissue. DPNS applying a dual ion beam (see FIG. 73) exposure with variant gas species and incidence angle (plasma orientation) is used to produce the inner-tube and outer-tube features.

Different strategies are planned and designed approaches in order to perform the desired modifications, the sequence of surface treatment is illustrated in FIG. 74. The focus will be in the used of two different gas species, neutral and reactive, which allows the topography and chemistry changes during the same process step. FIG. 74, illustrates the irradiation steps. The irradiation simultaneously with the two gas species or in sequential steps, one gas followed by the other. The incident angle is moderated by control of the piece direction. FIG. 74 shows a schematic representation of the different irradiation approaches by DPNS combining neutral and reactive gas species. A), left panel, shows the sequential irradiation process which uses one gas specie in each step (first Argon and after that oxygen). B) middle panel, shows simultaneously, process needs the combination of reactive and neutral gases but with different incidence angles. C), right panel, shows rolling in sequential steps, which is similar to a sequential irradiation, but moving the sample to change the irradiated surface.

As an example of this 3D tubular scaffold before irradiated with oxygen plasma is shown in FIG. 75. FIG. 75 shows SEM images of the raw surface of the silk tube scaffold. The initial surface shows an heterogeneous topography with pores at micro scale level as well smooth areas at the nanoscale order, covered the whole sample. As an example of this 3D tubular scaffold before irradiation with oxygen plasma is shown in FIG. 75. The control surface, without irradiation process, was deeply analyzed in both sides of the structure with the aim of the study the initial surface as seen in FIG. 76 and FIG. 77, as well as FIG. 78.

Moreover, these technologies show the capacity to modify any complex 3D geometry offering the modification of any material without any structure. DPNS process surface modifications on these 3D scaffolds were performed and the results are shown in FIG. 79. Notice the remarkable effect of irradiation using oxygen plasma in DPNS at surface topography changes between unmodified and modified silk structures. In FIG. 79, the extracted ions produced different nanofeatures with noticeable morphology depending on the incidence angle. Irradiation at normal angles, Silk 0 deg, reveals a porous surface in which higher magnification shows the nanofeatures are more similar to nanopillars or nanocolumns. These nanopillars seem to increase in size using off normal angles such as silk 45 degrees and with off normal oblique angles the nanopillars increased in length but became more close to each other, resulting in a smoother surface.

The 3D surface and the morphology of the irradiated topography was characterized deeply using AFM which the results obtained are shown in FIG. 79. The roughness of the control samples is small as expected, however, the Ra values increase in silk 0 deg (187.0 nm) which also corresponds to the 3D AFM image. In the case of oblique and highly oblique angles, the roughness decreases significantly, from 120.5 nm to 52.5 nm in 45 degree incidence angle and 60 degree incidence angle. The Ra are lower as the roughness square mean (RMS) which gives information about the length of the nanofeatures developed by DPNS.

FIG. 79. Notice the remarkable effect of irradiation using oxygen plasma in DPNS at surface topography changes between unmodified and modified silk structures. FIG. 79 shows SEM images of flat Silk scaffolds irradiated by DPNS. 3D tubular silk scaffolds were cut and flattened previous to the irradiation using DPNS. These flat Silk films shown different nanofeatures due to irradiation process using Oxygen gas species, energy of 500 eV, fluence of 5.0 E17 cm⁻² and different incidence angle (0°, 45°, and 60° degrees). Notice the size of the nanofeatures as well their orientation. The control silk (without any irradiation) revealed a pore like structure and smooth areas whereas the irradiated samples, showed a homogeneous modification of the whole structure with no pores and increased roughness. Increasing the incidence angle from 0° to 45° degrees increased the roughness and the morphology of these nanostructures change from nanocolumns to nanocolumns with sharp edges. These morphologies are observed in 60° as well but with a slight difference, the orientation of these nanofeatures. The angle produce a high surface modification with these nanocolumns close between them and with the bulk material, finding in some areas a smoother effect.

FIG. 80 shows AFM images and roughness (Ra and RMS) quantification of the silk scaffolds of FIG. 79. The analysis was performed using Gwydion software and three images were analyzed from each sample. Using another software to treat images, Image J, roughness values were obtained to compare and validate the AFM results. From Image J, we could corroborate the increase in roughness values, Ra and Rg, in the samples irradiated using normal incidence angles. Significant differences were observed for silk 60 deg (p 0.5) compared to silk 0 deg, in both Ra and Rg values. The oblique angles achieve a clear reduction and develop a smoother area, however, the nanopillars are still present. They seem to be more close between revealing a decreased width (25 nm) and a higher length (270 nm). The silk 45 deg showed medium values with nanopillars of 38 nm of width and 230 nm of length. Using normal incidence angles, silk 0 deg, the width was the highest (40 nm) and the smaller length close to 200 nm.

FIG. 81 shows quantitative data obtained through the analysis of SEM images by Image J. Width (Panel A) and length (Panel B) of the nanopillar was quantified using three images per condition and measuring 20 nanofeatures per image. Panel C and Panel D show the results obtained through Image J using plugin Roughness calculator. Even though the Ra and Rg are lower than those described by AFM, the roughness relationship corresponds to that found in AFM.

FIG. 82 shows an example of topography analysis using Image J. Evaluation of the nanofeatures height using 2D plots in the Software are shown in Panels A, B, and C. The middle and right panels show the equivalent SEM image of each sample (middle) to measure width and right panel shows 3D plots of the surface.

### REFERENCES

[1] Wang, S., Yaszemski, M. J., Knight, A. M., Gruetzmacher, J. A., Windebank, A. J., & Lu, L. (2009). Photo-Crosslinked Poly (ε-caprolactone fumarate) Networks for Peripheral Nerve Regeneration: Physical Properties and Preliminary Biological Evaluations. Acta Biomaterialia, 5(5), 1531-1542. http://doi.org/10.1016/j.actbio.2008.12.015
[2] Daly, W., Yao, L., Zeugolis, D., Windebank, A., & Pandit, A. (2011). A biomaterials approach to peripheral nerve regeneration: bridging the peripheral nerve gap and enhancing functional recovery. Journal of the Royal Society Interface, rsif20110438.
[3] R. López-Cebral, J. Silva-Correia, R. L. Reis, T. H. Silva, and J. M. Oliveira. Peripheral Nerve Injury: Current Challenges, Conventional Treatment Approaches, and New Trends in Biomaterials-Based Regenerative Strategies. ACS Biomaterials Science & Engineering. DOI: 10.1021/acsbiomaterials. 7b00655

### Example 5. Demonstration of Killing of E. coli by Bacterial Cellulose Treated by Methods of the Invention

Device-associated infections account for nearly half of all US healthcare-associated infections, limiting the performance and lifetime of biomedical materials. Those infections are commonly connected to the unwanted and non-specific adhesion of microbial or thrombotic agents to the surface of synthetic materials, known as a biofouling. Systemic antibiotic therapy is the gold standard for the treatment of biofouling events; however, it fails to achieve adequate antibiotic concentrations at the infected site, raising the risk of microbial resistance. The physicochemical properties of the bacterial cells and the substratum, as well as the environmental conditions, are known to govern the first stages of biofouling.

In nature, some insects (i.e., cicada and dragonfly) and animals (i.e., shark) have developed strategies to limit bacterial colonization in their wings and skin by extending sharp and complex nanotopographies. For example, hexagonal arrays of nanoscale pillars in the cicada wing are capable of penetrating the bacterial wall of gram-negative cells and kill them via a mechanism purely based on the surface structure of the wing. Approaches inspired by those examples in nature have explored the use of surface nanotopography as a route not only to limit bacterial attachment and biofilm build-up in biomedical devices but also to reduce the risk of microbial resistance. However, current approaches are limited by their lack of nanopattern variability and the lack of short- and long-range control of nanoscale ordering and surface chemistry, especially on clinically-relevant polymers.

Here, we have used Directed Plasma Nanosynthesis (DPNS) to nanopattern a fibrous hydrogel based on bacterial cellulose (BC) and provide with bactericidal properties this polymer against E. coli. BC is a biocompatible and degradable hydrogel synthesized by the bacterial strain Acetobacter xylinum, and commonly used as a dressing in wound healing (i.e., burned skin).

FIGS. 83(A) and 83(B) shows treatment of bacterial cellulose (BC) with Argon at a fluence of 1E18 ions/cm² creates nanopillars-like structures at the surface of the material. The left panel, at FIG. 83(A) shows that pristine BC is formed by ribbon-like fibers with an average diameter of 30 nm, and without well-defined pore structure; and the right panel, at FIG. 83(B) shows irradiation of the BC with Argon at 1000 eV, 0 degrees and at 1E18 ions/cm² generates nanopillars-like structures, which are uniformly distributed at the interface of the material. Images where taken using a Hitachi 4800 scanning electron microscope.

FIGS. 84 (A), 84(B), and 84(C) show that Argon-treated BC is superhydrophilic, rich in C—C/C—H bonds, and has a Young Modulus of 8.77 MPa. FIG. 84(A) shows that the effective Young Modulus of the Argon-treated BC increased (8.77 MPa) compare to that of the pristine BC (4.86 MPa), but this value was smaller than the reported for Dragonfly (>20 MPa) and Cicada wings (3.7 GPa) nanopillars. FIG. 84A is performed in aqueous solution, and demonstrates further that the nanostructures, after synthesis, maintain their elastic structure even in liquid media. The hydrogels irradiated by DPNS keep their nanofeatures even in liquid media, after contact with media. The nanofeatures are stable in air or in hydrophilic media. In FIG. 84(B) shows the XPS-survey for the C1s spectra of pristine BC (pre) and Argon-treated BC (post) shows that the peak corresponding to C—C/C—H bonds increased dramatically while those corresponding to C—O—C/C—OH and C═O/O—C—O bonds decreased. The COO— bonds remained similar. These may indicate a preferential sputtering of oxygen with removal C—O bonds and a restructure of the polymer chains. FIG. 84(C) Argon-treated BC has a water contact angle equal to zero, indicating that is superhydrophilic. Water contact angle for glass and pristine BC are provided for comparison.

FIGS. 85(A) and 85(B). Argon-treated BC shows bactericidal activity against E. coli. Scanning electron images of E. coli exhibiting a normal cylindrical shape of about 2 urn on pristine BC in FIG. 85(A). Argon-treated BC is incubated in liquid media with E. coli. When grown on Argon-treated BC, E. coli appears flat with damaged and broken bodies typical of a dead cell in FIG. 85(B). Images in the bottom are magnification of the upper panels. E. coli was incubated for 1 hour at 37 C and 100 rpm, fixed with formalin, dehydrated in serial dilutions of ethanol, and then dried at room temperature in a desiccator connected to vacuum. Images where taken using a Hitachi 4800 scanning electron microscope. The experiment demonstrates that the hydrogels irradiated by DPNS keep their nanofeatures even in liquid media, after contact with media. The nanofeatures are stable in air or in hydrophilic media, and retain their functionality, e.g., ability to kill bacteria.

### REFERENCES

Bayramov, D. F., & Neff, J. A. (2017). Beyond conventional antibiotics—New directions for combination products to combat biofilm. Advanced drug delivery reviews, 112, 48-60
Harding, Jacqueline L., and Melissa M. Reynolds. “Combating medical device fouling.” Trends in biotechnology 32, no. 3 (2014): 140-146
Tripathy, A., et al. (2017). Natural and Bioinspired nanostructured bactericidal surfaces.
Crawford, Russell J., et al. “Surface topographical factors influencing bacterial attachment.” Advances in colloid and interface science 179 (2012): 142-149.
Ivanova et al. 2012. Natural bactericidal surfaces: mechanical rupture of Pseudomonas aeruginosa cells by cicada wings. Small
Bandara, Chaturanga D., et al. “Bactericidal effects of natural nanotopography of dragonfly wing on Escherichia coli.” ACS applied materials & interfaces 9.8 (2017): 6746-6760.

### Example 6. PDMS Nanopatterning

FIG. 86. Wrinkle structure in Argon-treated PDMS depends on the ion energy, and it is irrespective of the initial material stiffness. Experimental samples were prepared by mixing silicone at ratios of 5:1, 10:1, and 30:1 of the base-to-catalyst agent (Sylgard-184, Dow Corning) and then spin-coated on glass slides at 1500 rpm for 1 min, and cured at room temperature for at least 24 h. Experimental samples were subsequently irradiated with Argon, at normal incidence, 1E17 ions/cm², and at ion energies of 1000 eV and 500 eV. The effective Young Modulus of the initial samples was measured using a Piuma Nanoindenter with colloidal probes of 9 um in diameter and stiffness of 44 N/m. The images were obtained in an Asylum Cypher AFM operating in tapping mode.

FIG. 87(A), 87(B), and 87(C). The wavelength of wrinkle structures in irradiated PDMS can be tuned by controlling the angle of incidence and the initial stiffness of the material. FIG. 87(A) AFM images of PDMS prepared at ratios of 10:1, 30:1, and 50:1 of the base-to-catalyst agent show that the wrinkle wavelength decreases with the angle of incidence increases. FIG. 87(B) Power spectral density for AFM images in 87(A), and showing that the spatial frequency of the wrinkles depends on the initial stiffness of the PDMS. FIG. 87(C) The RMS height remains similar at an angle of incidence of 0 and 45 degrees for rigid PDMS, but increases for soft substrates at normal incidence. The Effective Young Modulus is modulus is independent of the incidence angle.

FIGS. 88(A), 88(B), and 88(C). Wrinkle structure and water contact angle are independent of the ion species implanted, but the Young Modulus shows dependence on the ion species. FIG. 88(A) Scanning electron microscope images showing a similar wrinkle structure on a film of PDMS prepared with a base to catalyst ratio of 10:1. Experimental sample was irradiated with argon, oxygen, and krypton at 0 and 45 degrees of incidence angle, keeping constant the ion fluence and energy at 1000 eV and 1E17 ions/cm². FIG. 88(B) Effective Young Modulus for the experimental samples in 88(A) shows a statistically significant difference between argon versus oxygen and krypton irradiation of PDMS(10:1) (*p-value <0.01). FIG. 88(C) Water contact angle for PDMS(10:1) treated with argon, oxygen, and krypton at two incidence angles. The pristine sample exhibits a spherical water drop indicative of a low interaction with the liquid, but after irradiation, the water contact angle decreases almost by two-fold, and it is similar among the different ion species. Water contact angle measurements were performed in a Rame-Hart Contact Angle Goniometer using 2 ul of ultrapure water.

FIG. 89 shows the bacteria behavior attached on silk treated by DPNS using Oxygen ion beam and different incidence angle. The different nanofeatures (their size, morphology and chemistry surface) have a great influence in bacteria viability. In these images E. coli in healthy state shows a round and elongate shape, which can be found in pristine sample whereas in Silk−0°, Silk 45° and Silk 60° the majority of the E. coli attached are dying, observing some leaks of their content (see yellow arrows) in which the membrane is disrupted and broke leading to dead bacteria.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A polymer composition comprising:
a polymer substrate having a surface;
a plurality of metal, metal oxide, or carbon allotrope nanoparticles disposed on said surface;
wherein said surface has a plurality of nanoscale domains characterized by a surface geometry providing a selected function;
wherein each of said nanoscale domains has at least one lateral spatial dimension selected over the range of 10 nm to 1 μm and a vertical spatial dimension less than 200 nm.

2. A polymer composition comprising:
a polymer substrate having a surface;
a plurality of metal, metal oxide nanoparticles, or carbon allotrope nanoparticles disposed on said surface;
wherein said surface has a plurality of nanoscale domains characterized by a surface geometry providing a selected function;
wherein said nanoscale domains are generated by exposing said surface to one or more directed energetic particle beam characterized by one or more beam properties.

3. The polymer of claim 1 or 2, wherein the polymer is a polysaccharide biopolymer.

4. The polymer of claim 1 or 2, wherein the polymer is a synthetic polymer.

5. The composition of any of claims 1-4, wherein said selected function is an activity related to at least one biological or physical property, relative to a polymer composition not having said plurality of nanoscale domains characterized by said nanofeatured surface geometry.

6. The composition of claim 5, wherein said activity is an enhancement of a biological property selected from the group consisting of cell adhesion activity, cell proliferation activity, cell in-migration activity, cell differentiation activity, anti-bacterial activity, bactericidal activity, anti-inflammatory activity, osseointegration activity, osseoconduction activity, osseoinduction activity, reduction of immunoresponse, and combinations thereof.

7. The composition of claim 6, wherein said enhancement of the biological property is equal to or greater than about 100% to about greater than or equal to 500%.

8. The composition of claim 6, wherein said biological property is anti-bacterial activity or bactericidal activity.

9. The composition of claim 8, wherein said enhancement of anti-bacterial activity and bactericidal activity is greater than or equal to 100%.

10. The composition of claim 6, wherein said biological property is cell adhesion activity, proliferation activity or in-migration activity, and the enhancement is greater than or equal to 100%.

11. The composition of any of claims 1-10, wherein the composition has increased hemocompatibility.

12. The composition of claim 11, wherein the increase in hemocompatibility is greater than or equal to 50%.

13. The composition of claim 5, wherein said activity is an enhancement of a physical property selected from the group consisting of surface hydrophilicity, surface free energy, surface hydrophobicity, sensing, drug transport, surface acidity, surface basicity, and combinations thereof.

14. The composition of any of claims 1-13, wherein said surface geometry is spatial distribution of relief features, recessed features, localized regions characterized by a selected composition, phase, crystallographic texture, or any combination of these.

15. The composition of any of claims 1-4, wherein said surface geometry is a periodic or semi-periodic spatial distribution of said nanoscale domains.

16. The composition of any of claims 1-4, wherein said surface geometry is a selected topology, topography, morphology, texture or any combination of these.

17. The composition of claim 3, wherein said nanoscale domains comprise nanopillars, nanowalls, nanorods, nanoplates, nanoripples, surface porous structure, or any combination thereof having lateral spatial dimensions selected over the range of 10 nm to 1 μm and vertical spatial dimensions of less than or equal to 200 nm and wherein said nanoscale domains are separated from one another by a distance of 50-500 nm.

18. The composition of claim 17, wherein said nanopillars, nanowalls, nanorods, nanoplates, nanoripples, surface porous structure, or combination thereof, are inclined towards a direction oriented along a selected axis relative to said surface.

19. The composition of claim 17, wherein the metal or metal oxide nanoparticles comprise gold nanoparticles, silver nanoparticles, zinc sulfide nanoparticles, zinc oxide nanoparticles, copper nanoparticles, platinum nanoparticles, cobalt nanoparticles, cobalt ferrite nanoparticles, ferric oxide nanoparticles, yttrium nanoparticles, zirconium nanoparticles, ruthenium nanoparticles, palladium nanoparticles, or any combinations thereof.

20. The composition of claim 17, wherein the polysaccharide biopolymer is selected from the group consisting of cellulose wherein the cellulose is selected from the group consisting of bacterial nanocellulose, nanocellulose, and a cellulose derivative; chitin; a dextran; chitosan; and combinations thereof.

21. The composition of claim 20, wherein the nanoscale domains comprise nanopillars; wherein said polysaccharide biopolymer comprises chitosan or bacterial nanocellulose; wherein said metal, metal oxide, or carbon allotrope nanoparticles comprise zinc sulfide nanoparticles, gold nanoparticles or silver nanoparticles; and wherein said selected function is enhanced antibacterial properties or enhanced hydrophilicity.

22. The composition of claim 21, wherein said nanopillars have lateral spatial dimensions selected over the range of 10 nm to 1 μm and vertical spatial dimensions of less than or equal to 200 nm and wherein said nanoscale domains are separated from one another by a distance of 50-500 nm, and wherein the nanoparticles have a diameter of between about 10-50 nm.

23. The composition of claim 20, wherein the nanoscale domains comprise surface porous structure; wherein said polysaccharide biopolymer comprises chitosan or bacterial nanocellulose; wherein said metal, metal oxide, or carbon allotrope nanoparticles comprise zinc sulfide nanoparticles, gold nanoparticles or silver nanoparticles; and wherein said selected function is enhanced antibacterial properties or enhanced hydrophilicity.

24. The composition of claim 23, wherein said surface porous structure has lateral spatial dimensions selected over the range of 50 nm to 500 μm and vertical spatial dimensions of between 10 and 50 nm and wherein the nanoparticles have a diameter of between about 10-50 nm.

25. The composition of claim 4, wherein said nanoscale domains comprise nanoripples having lengthwise spatial dimensions selected over the range of 0.5 microns to 10 microns, vertical dimension of between 50 nm to about 200 nm, peak to peak spatial dimensions of between about 100 nm to 300 nm, and wherein the nanoparticles have a diameter of between about 10 and 50 nm and/or between 250-500 nm.

26. The composition of claim 25, wherein the metal or metal oxide nanoparticles comprise gold nanoparticles, silver nanoparticles, zinc sulfide nanoparticles, zinc oxide nanoparticles, copper nanoparticles, platinum nanoparticles, cobalt nanoparticles, cobalt ferrite nanoparticles, ferric oxide nanoparticles, yttrium nanoparticles, zirconium nanoparticles, ruthenium nanoparticles, palladium nanoparticles, or any combinations thereof.

27. The composition of claim 26, wherein the metal or metal oxide nanoparticles comprise zinc oxide nanoparticles.

28. The composition of claim 25, wherein said synthetic polymer is selected from the group consisting of a polyolefin; a silicone; a polyacrylate or polymethacrylate; a polyester; a polyether; a polyamide, and a polyurethane.

29. The composition of claim 28, wherein the synthetic polymer is a polyolefin selected from the group consisting of polypropylene, polyethylene, poly(tetrafluoroethylene) and poly(vinyl chloride); or wherein the synthetic polymer is a silicone and comprises poly(dimethyl siloxane); or wherein the synthetic polymer is a polyacrylate selected from the group consisting of poly(methyl methacrylate), poly(hydroxyethyl methacrylate); or wherein the synthetic polymer is a polyester selected from the group consisting of poly(ethylene terephthalate), poly(glycolic acid), poly-lactic acid, polydioxanone; or wherein the synthetic polymer is a polyether selected from the group consisting of polyether ether ketone and polyether sulfone.

30. The method of claim 29 wherein said nanoscale domains comprise nanoripples; wherein said polymer comprises poly(dimethyl siloxane); wherein said metal, metal oxide, or carbon allotrope nanoparticles comprise zinc oxide nanoparticles; and wherein said selection function is enhanced hydrophilicity.

31. The method of claim 30, wherein said nanoripples having lengthwise spatial dimensions selected over the range of 0.5 microns to 10 microns, vertical dimension of between 50 nm to about 200 nm, peak to peak spatial dimensions of between about 100 nm to 300 nm, and wherein the nanoparticles have a diameter of between about 10 and 50 nm and/or between 250-500 nm.

32. The composition of any of claims 1-31, wherein said polymer composition comprises a component of a medical device, a sensor, a catalyst, or an imaging system.

33. The composition of claim 32, wherein said medical device is a surgical material, an implant, a catheter, a wound suture, an artificial tendon, a pacemaker, a cochlear implant, a neural implant, intravenous tubing, a surgical sponge, gauze, a needle, a syringe, a cosmetic silicone implant, a coating, a connection, a wire, or a cosmetic silicone prosthetic.

34. The composition of any of claims 1-31, wherein said polymer composite comprises a component of a surface, a tank, a conveyor, a floor, a drain, a cooler, a freezer, an equipment surface, a wall, a valve, a belt, a pipe, an air conditioning conduit, a cooling apparatus, a food or drink dispensing line, a heat exchanger, a boat hull, a dental waterline, an oil drilling conduit, a contact lens, or a storage case.

35. The composition of claim 2, wherein the directed energetic particle beam is a broad beam, focused beam, asymmetric beam, thermalized plasma in liquid or any combination of these.

36. The composition of claim 2, wherein said one or more beam properties is intensity, fluence, energy, flux, incident angle, ion composition, neutral composition or any combinations thereof.

37. The composition of claim 2, wherein said directed energetic particle beam comprises one or more ions, neutrals or combinations thereof.

38. A method of fabricating the polymer composition of claim 3, said method comprising:
providing a polysaccharide biopolymer, wherein the polysaccharide biopolymer is in a solution or is a dried film;
providing a metal salt in solution to the polysaccharide biopolymer;
incubating the solution comprising the metal salt and polysaccharide biopolymer;
drying the solution, forming a substrate having a surface; and
directing a directed energetic particle beam onto said dried substrate surface, thereby generating a plurality of nanoscale domains and metal nanoparticles on said surface;
wherein said directed energetic particle beam has one or more beam properties selected to generate said plurality of nanoscale domains characterized by a surface geometry providing a selected function.

39. The method of claim 38, further comprising: immersing the dried substrate in a liquid, wherein the directed energetic particle beam is directed onto said substrate surface through the liquid.

40. The method of claim 38 or 39, wherein the directed energetic particle beam is a broad beam, focused beam asymmetric beam or any combination of these.

41. The method of claim 38 or 39, wherein said step of directing said directed energetic particle beam onto said substrate surface comprises directed irradiation synthesis (DIS), directed plasma nanosynthesis (DPNS), Direct Seeded Directed Plasma Nanosynthesis (DSDPNS), DSPNS (directed soft plasma nanosynthesis) or any combination of these.

42. The method of claim 38 or 39, wherein said one or more beam properties is intensity, fluence, energy, flux, incident angle, ion composition, neutral composition or any combinations thereof.

43. The method of claim 38 or 39, wherein said directed energetic particle beam comprises one or more ions, neutrals or combinations thereof.

44. The method of claim 43, wherein said ions are krypton ions, argon ions, oxygen ions, or a combination thereof.

45. The method of claim 42, wherein said one or more beam properties comprise incident angle and said incident angle is selected from the range of 0° to 90°.

46. The method of claim 42, wherein said one or more beam properties comprise fluence and said fluence is selected from the range of 1×10¹⁶ ions/cm² to 1×10¹⁹ ions/cm².

47. The method of claim 38, wherein said one or more beam properties comprise energy and said energy is selected from the range of 0.1 keV to 10 keV.

48. The method of claim 38 or 39, wherein the metal salt is selected from the group consisting of HAuCl₄ and AgNO₃.

49. The method of claim 38 or 39, wherein said polysaccharide biopolymer is selected from the group consisting of cellulose, nanocellulose, a cellulose derivative, chitin, a dextran, chitosan, or combinations thereof.

50. The method of claim 38, wherein said nanoscale domains comprise nanopillars wherein said nanopillars have lateral spatial dimensions selected over the range of 10 nm to 1 μm and vertical spatial dimensions of less than or equal to 200 nm and wherein said nanoscale domains are separated from one another by a distance of 50-500 nm, and wherein the nanoparticles have a diameter of between about 10-50 nm.

51. The method of claim 38, wherein the nanoscale domains comprise nanopillars; wherein said polysaccharide biopolymer comprises chitosan or bacterial nanocellulose; wherein said metal, metal oxide, or carbon allotrope nanoparticles comprise zinc sulfide nanoparticles, gold nanoparticles or silver nanoparticles; and wherein said selected function is enhanced antibacterial properties or enhanced hydrophilicity.

52. The method of claim 39, wherein the nanoscale domains comprise surface porous structure; wherein said polysaccharide biopolymer comprises chitosan or bacterial nanocellulose; wherein said metal, metal oxide, or carbon allotrope nanoparticles comprise zinc sulfide nanoparticles, gold nanoparticles or silver nanoparticles; and wherein said selected function is enhanced antibacterial properties or enhanced hydrophilicity.

53. The method of claim 39, wherein said surface porous structure has lateral spatial dimensions selected over the range of 50 nm to 500 μm and vertical spatial dimensions of between 10 and 50 nm and wherein the nanoparticles have a diameter of between about 10-50 nm.

54. A method of fabricating a polymer composition of claim 4, said method comprising:
providing a synthetic polymer substrate;
providing a solid metal or metal oxide source target material;
directing a directed energetic particle beam onto said target material surface, thereby generating a sputtered beam of target material directed onto the surface of the synthetic polymer substrate;
directing a second directed energetic particle beam onto said substrate surface, thereby generating a plurality of nanoscale domains and metal or metal oxide nanoparticles on said substrate surface;
wherein said directed energetic particle beams have one or more beam properties selected to generate said plurality of nanoscale domains characterized by a surface geometry providing a selected function.

55. The method of claim 54, wherein the first or second directed energetic particle beam is independently a broad beam, focused beam asymmetric beam or any combination of these.

56. The method of claim 54, wherein said step of directing said directed first or second energetic particle beam onto said substrate surface comprises directed irradiation synthesis (DIS), directed plasma nanosynthesis (DPNS), Direct Seeded Directed Plasma Nanosynthesis (DSDPNS), DSPNS (directed soft plasma nanosynthesis) or any combination of these.

57. The method of claim 54, wherein said one or more beam properties is intensity, fluence, energy, flux, incident angle, ion composition, neutral composition or any combinations thereof.

58. The method of claim 54, wherein said directed energetic particle beam comprises one or more ions, neutrals or combinations thereof.

59. The method of claim 54, wherein said ions are krypton ions, argon ions, or oxygen ions.

60. The method of claim 54, wherein said one or more beam properties comprise incident angle and said incident angle is selected from the range of 0° to 90°.

61. The method of claim 54, wherein said one or more beam properties comprise fluence and said fluence is selected from the range of 1×10¹⁶ ions/cm² to 1×10¹⁹ ions/cm².

62. The method of claim 54, wherein said one or more beam properties comprise energy and said energy is selected from the range of 0.1 keV to 10 keV.

63. The method of claim 54, wherein the target is a metal target selected from the group consisting of zinc, gold, silver, copper, platinum, cobalt, cobalt, yttrium, zirconium, ruthenium, palladium, or any combinations thereof

64. The method of claim 54, wherein the synthetic polymer is a polyolefin selected from the group consisting of polypropylene, polyethylene, poly(tetrafluoroethylene) and poly(vinyl chloride); or wherein the synthetic polymer is a silicone and comprises poly(dimethyl siloxane); or wherein the synthetic polymer is a polyacrylate selected from the group consisting of poly(methyl methacrylate), poly(hydroxyethyl methacrylate); or wherein the synthetic polymer is a polyester selected from the group consisting of poly(ethylene terephthalate), poly(glycolic acid), poly-lactic acid, polydioxanone; or wherein the synthetic polymer is a polyether selected from the group consisting of polyether ether ketone and polyether sulfone.

65. The method of claim 54 wherein said nanoscale domains comprise nanoripples; wherein said polymer comprises poly(dimethyl siloxane); wherein said metal, metal oxide, or carbon allotrope nanoparticles comprise zinc oxide nanoparticles; and wherein said selection function is enhanced hydrophilicity, wherein said nanoripples have lengthwise spatial dimensions selected over the range of 0.5 microns to 10 microns, vertical dimension of between 50 nm to about 200 nm, peak to peak spatial dimensions of between about 100 nm to 300 nm, and wherein the nanoparticles have a diameter of between about 10 and 50 nm and/or between 250-500 nm.

66. A polymer composition comprising:
a polymer substrate having a surface; wherein said surface has a plurality of nanoscale domains characterized by a surface geometry providing a selected function;
wherein each of said nanoscale domains has at least one lateral spatial dimension selected over the range of 3 nm to 1 μm and a vertical spatial dimension less than 500 nm.

67. A polymer composition comprising:
a polymer substrate having a surface; wherein said surface has a plurality of nanoscale domains characterized by a surface geometry providing a selected function;
wherein said nanoscale domains are generated by exposing said surface to one or more directed energetic particle beam characterized by one or more beam properties.

68. The composition of claim 66 or 67, wherein said function is an activity related to at least one biological or physical property, relative to a polymer composition not having said plurality of nanoscale domains characterized by said nanofeatured surface geometry.

69. The composition of claim 68, wherein said activity is enhancement of a biological property selected from the group consisting of cell adhesion activity, cell proliferation activity, cell in-migration activity, cell differentiation activity, anti-bacterial activity, bactericidal activity, anti-inflammatory activity, osseointegration activity, osseoconduction activity, osseoinduction activity, reduction of immunoresponse, and combinations thereof.

70. The composition of claim 69, wherein said biological property is selected from the group of enhancement of cell adhesion activity, enhancement of cell proliferation activity, enhancement of anti-bacterial activity, and increased hydrophilicity; and the enhancement of function or activity is equal to or greater than 100%.

71. The composition of claim 69, wherein said activity is an enhancement of a physical property selected from the group consisting of surface hydrophilicity, surface free energy, surface hydrophobicity, sensing, drug transport, surface acidity, surface basicity, and combinations thereof.

72. The composition of claim 66 or 67, wherein said surface geometry is spatial distribution of relief features, recessed features, localized regions characterized by a selected composition, phase, crystallographic texture, or any combination of these.

73. The composition of claim 66 or 67, wherein said surface geometry is a periodic or semi-periodic spatial distribution of said nanoscale domains.

74. The composition of claim 66 or 67, wherein said surface geometry is a selected topology, topography, morphology, texture or any combination of these.

75. The composition of claim 66 or 67, wherein each of said nanoscale domains are characterized by a vertical spatial dimension of between 50 nm and 1000 nm.

76. The composition of claim 66 or 67, wherein each of said nanoscale domains are characterized by a vertical spatial dimension selected over the range of 200 nm to 300 nm.

77. The composition of claim 66 or 67, wherein said nanoscale domains comprise nanopillars, nanowalls, nanorods, nanoplates, nanoripples, surface porous structure, or any combination thereof having lateral spatial dimensions selected over the range of 10 nm to 1 μm and vertical spatial dimensions of less than or equal to 200 nm and wherein said nanoscale domains are separated from one another by a distance of 50-500 nm.

78. The composition of claim 77, wherein said nanoscale domains comprise nanopillars or nanocolumns which are inclined towards a direction oriented along a selected axis relative to said surface.

79. The composition of claim 77, wherein said nanoscale domains comprise nanoripples.

80. The composition of claim 66 or 67, wherein said polymer substrate is a fibrous protein polymer substrate, a polysaccharide biopolymer substrate, or a synthetic polymer substrate.

81. The composition of claim 80, wherein said polymer substrate is a fibrous protein substrate selected from the group of silk fibroin, collagen, elastin, and keratin.

82. The composition of claim 66 or 67, wherein said polymer composition comprises a component of a medical device.

83. The composition of claim 67, wherein the directed energetic particle beam is a broad beam, focused beam, asymmetric beam, reactive beam or any combination of these.

84. The composition of claim 67, wherein said one or more beam properties is intensity, fluence, energy, flux, incident angle, ion composition, neutral composition, ion to neutral ratio or any combinations thereof.

85. A method of fabricating a polymer substrate composition providing a selected function, said method comprising:
providing a polymer substrate having a substrate surface; and
directing a directed energetic particle beam onto said substrate surface, thereby generating a plurality of nanoscale domains on said surface;
wherein said directed energetic particle beam has one or more beam properties selected to generate said plurality of nanoscale domains characterized by a surface geometry providing the selected function.

86. The method of claim 85, wherein said selected function is an activity related to at least one biological or physical property, relative to a polymer composition not having said plurality of nanoscale domains characterized by said nanofeatured surface geometry.

87. The method of claim 86, wherein said activity is an enhancement of a biological property selected from the group consisting of cell adhesion activity, cell proliferation activity, cell in-migration activity, cell differentiation activity, anti-bacterial activity, bactericidal activity, anti-inflammatory activity, osseointegration activity, osseoconduction activity, osseoinduction activity, reduction of immunoresponse, and combinations thereof.

88. The method of claim 87, wherein said biological property is selected from the group of enhancement of cell adhesion activity, enhancement of cell proliferation activity, enhancement of anti-bacterial activity; and the enhancement of function or activity is equal to or greater than 100%.

89. The method of claim 86, wherein said activity is an enhancement of a physical property selected from the group consisting of surface hydrophilicity, surface free energy, surface hydrophobicity, sensing, drug transport, surface acidity, surface basicity, and combinations thereof.

90. The method of claim 85, wherein said surface geometry is spatial distribution of relief features, recessed features, localized regions characterized by a selected composition, phase, crystallographic texture, or any combination of these.

91. The method of claim 85, wherein said surface geometry is a periodic or semi-periodic spatial distribution of said nanoscale domains.

92. The method of claim 85, wherein said surface geometry is a selected topology, topography, morphology, texture or any combination of these.

93. The method of claim 85, wherein each of said nanoscale domains are characterized by a vertical spatial dimension of between 150 nm and 500 nm.

94. The method of claim 85, wherein each of said nanoscale domains are characterized by a vertical spatial dimension selected over the range of 200 nm to 300 nm.

95. The method of claim 85, wherein said nanoscale domains comprise nanopillars, nanowalls, nanorods, nanoplates, nanoripples, surface porous structure, or any combination thereof having lateral spatial dimensions selected over the range of 10 nm to 1 μm and vertical spatial dimensions of less than or equal to 200 nm and wherein said nanoscale domains are separated from one another by a distance of 50-500 nm.

96. The method of claim 95, wherein said nanoscale domains comprise nanopillars or nanocolumns which are inclined towards a direction oriented along a selected axis relative to said surface.

97. The method of claim 95, wherein said nanoscale domains comprise nanoripples.

98. The method of claim 85, wherein said polymer substrate is a fibrous protein polymer substrate, a polysaccharide biopolymer substrate, or a synthetic polymer substrate.

99. The method of claim 98, wherein said polymer substrate is a fibrous protein substrate selected from the group of silk fibroin, collagen, elastin, and keratin.

100. The method of claim 85, wherein said polymer substrate composition comprises a component of a medical device.

101. The method of claim 85, wherein the directed energetic particle beam is a broad beam, focused beam asymmetric beam or any combination of these.

102. The method of claim 85, wherein said step of directing said directed energetic particle beam onto said substrate surface comprises directed irradiation synthesis (DIS), directed plasma nanosynthesis (DPNS), Direct Seeded Directed Plasma Nanosynthesis (DSDPNS), DSPNS (directed soft plasma nanosynthesis) or any combination of these.

103. The method of claim 85, wherein said one or more beam properties is intensity, fluence, energy, flux, incident angle, ion composition, neutral composition ion to neutral ratio or any combinations thereof.

104. The method of claim 85, wherein said directed energetic particle beam comprises one or more ions, neutrals or combinations thereof.

105. The method of claim 104, wherein said ions are krypton ions, argon ions, oxygen ions, or a combination thereof.

106. The method of claim 103, wherein said one or more beam properties comprise incident angle and said incident angle is selected from the range of 0° to 80°.

107. The method of claim 103, wherein said one or more beam properties comprise fluence and said fluence is selected from the range of 1×10¹⁶ cm⁻² to 1×10¹⁹ cm⁻².

108. The method of claim 103, wherein said one or more beam properties comprise energy and said energy is selected from the range of 0.01 eV to 10 keV.

109. The method of claim 85, wherein the polymer substrate composition retains the surface geometry providing the selected function after an incubation step in a liquid media.

110. The method of claim 109, wherein the selected function is anti-bacterial activity.

111. The composition of claim 66 or 67, wherein the polymer composition retains the surface geometry providing the selected function after an incubation step in a liquid media.

112. The composition of claim 111, wherein the selected function is anti-bacterial activity.
